SEQUENCE LISTING (170 sequences)

SEQ ID NO: 1 - DNA, 249 bp, Pseudomonas aeruginosa
atgaaagcaatgaagcaacgcatcgccaagttcagcccggtcgcctcgttccgcaacctgtgcatcgccggttccgtcactgccgcgacttcgctgccggccttcgccggggtgatcgacaccagcgcggtggaatcggcgatcaccgatggccagggcgatatgaaggccattggcggctacatcgtcggcgccctggtgatcctggccgttgccggcctgatctacagcatgttgcgcaaggcgtaa

SEQ ID NO: 2 - DNA, 252 bp, Pseudomonas aeruginosa
atgtcaggcgttgtcgctgtgcaggtgtgtaccgcgtggacctcgacccccgagggcttcatggcgtgtcgcgaactcgcatggcaacaggcctacctgattccgccagaggccgctggatacgtggacatcctggtcaacggtggtttctccccggaagccttcggcatcggtgccgctggcgtcctgggatcgttcgtgacggggcttttgattggctgggtcgcgtcacttcttcgtaaagccaagtag

SEQ ID NO: 3 - DNA, 435 bp, Pseudomonas aeruginosa
atgaacatgtttgcaacccaaggcggcgtcgtcgaactgtgggtcaccaagaccgacacctatacctcgaccaagaccggggaaatctacgcctcggtccaatccatcgccccgatcccggaaggtgcccgtggcaacgccaagggattcgagatcagcgaatacaacatcgagccgaccctgctggacgccatcgtcttcgaaggccagccggtgctctgcaagttcgccagcgtggtccgtccgacccaagaccgtttcggccggatcaccaatacccaggtcctagtggatctgctggccgtgggcggcaagccgatggcgccgaccgcccaagccccggcccgcccgcaagtgcaggcccaagccccgcgcccggcccagcagccgcagggccaggacaaacaagacaagaccccggacgccaaggcgtaa

SEQ ID NO: 4 - DNA, 93 bp, Pseudomonas aeruginosa
atgctccgctatctctcgctgttcgcggtaggtctggccaccggctacgcctggggctggatcgacggcctagcggcctccctggctgtttga

SEQ ID NO: 5 - DNA, 291 bp, Pseudomonas aeruginosa
atggccgccagtccctactacctacgccaaacccacgccccggactgcgcctgctctgtgtgctggtccgcaaggcaggtcatcccattgcacagcccgtcgccgtgtccagactgtcggccccctgggctgccctatctggaaggtggccgctggctctgccgtccccgttccttctgcgcgaaacacgacccgtcccggcgtccgccgaagtactggcacgttgtgtacgacagcgggaagcccacgccctttgtgcccgtgcgcgaagcattccaactggagggctga

SEQ ID NO: 6 - DNA, 1293 bp, Pseudomonas aeruginosa
atgaagaaaatcagccatcaaattcgcgtcagtatcgagtcggacggtcaggtcttggaaagcccgaaagggcggttgttcttcgacgacaccacggctcaattcaccgacctgtcaggcgtgcgcattctgcggtgcggcgtggatacggtgcggcagttgtacaacggcaaactccggccggaagtcatggcgctgtttgacctctcggtggatgtggtcgagttcgccggctacgagtggtccaagggccgcatcggtcgcgactccggctatcagtaccgcctgcagaacgctgaaatgggtctgatcctgctaatcaagaatcacaacatcaaggtcgacaccattggctcgcacctcaagatcgaggtatcgcctcacgccctcgatggcgccgatccgcgcatcctccagggcgtgctggatgatttggccgctgccgtgctgagtcactgcgaaaccaaccaagccgctgtgcatatcgccttggacgtgcagggctggaaaccgcctcgcgatctggtggaccgcatgcattgtcgctcgcgtcgggtgcgacaaatcagtgggatcgagcggatcgaattcgacggcaacgcctcggtctacgggcgtggcgagacgtacatgttcggctcggccaacggcctgcaactgtcgatctataacaagaccctccaggctcgggccaccgacaagctcgactattgggaaagcgtgtgggcgaccctgaacggggatccgttcggcgatggcgacccggcctataaccccctggaaacggtctggcggctcgaattccgcttccatcactccatcgtccagcagttctccgaaggctcgcgcatggcttcgggagaggtcattggctgccgcacctatgaggggctttgcccgcacctgcaaggactgtggaactacgcctgcgaaagcttcaagctgctgagccggacggcggtctacgatccgttctggagcctgatcagccaggacgcccgcgtccaggtcgagtgcgatccgctgatcgagcgcaccgagtatcggcgctattacaagaccgccaagggctttagcgggcgtaactgcgagatgtttctcggccagttcgtgagcctgatcgcgcgggagcgtgtcccggcaaaaaaggctattgagtccgcccgtaaactggagttctggcacgttatcgaagaccactatctcgccaagggttggactcgtcgcgatctggaaaggcacatacacaagctgatgtgtgatcggtatctgcggcgggggtatgccgtctaa

SEQ ID NO: 7 - DNA, 1263 bp, Pseudomonas aeruginosa
gtgtggggtcttacgatgaagtttgcgagcctgattctgatgcttctctttgccacggtggcgagggctgaggattactactggaaaattcagtcactgcctgaacgcttttcttcgccctcggcagcttgcgcggcgtgggccaaagccacgggacgccctggggagttcaccttcaccgggtctatgaaagcccgtgaccagacctcgttttggtgcgagttcacgaacaacgaaaccggcaagactgctgccgggtatggtcctgccggacgctatggcgatagctgtcccgaggggacggaatacgataaggcgaccggggtttgtaagtcgcctccgcaagaatgcaaggaaggcgaactgttcccggccaaaggcccggactctcccgtggttacctcgggaggccgtaactatgtcggtgacggcggcgccccgaccgcctgctatcaaagctgtgagtatggcggcaatcccagcccggccagttgctatctggtcaaaggctccaccacgaccggcttctgcaattacatcctcaagggcaccggacagaattgcggtgccgattcctacaccttctcccagaccggcgattcgctgaacccgcccgacactccgaacaccgatccttccgacccgaacgaccccggctgcccgcccggctggtcgtggtcgggaactacctgcgtcaaggccccgaccgatcccacggatccaaccgacccgaccacgccgggcagtgacggcggcggcgatggcaatggcggtggaaacaataacggcggcggcaatgacggcggcaccggcaatggcggcgacggcagcgggggaggggacggcaacggcgggggcgatggtagcggcgacggtgacggcagcggcacgggcggcgatggcaacggcacctgcgacccggcgaaagagaactgctccaccggccccgaaggccccggcggcgaactcaaggagcccacgcccggcacctgggatgacgccatcgccacctgggaaaagaaggtcgaggacgccaagcaagaactcaagaccaaggtgaaggccaacgtcgatcagatgaagggcgccttcgacctcaacctggcggaaggcggcgggcaactgccctgcgagtccatgaccatttggggcaagtcctactccctctgtatctccgactacgccggccaactctccagcctgcgcgtggcgctgctgctgatggccgcgctgatcgccgccctcattctgctgaaggactga

SEQ ID NO: 8 - DNA, 357 bp, Pseudomonas aeruginosa
atggaatggctctccggttttctcgatcagatcatcgccttcttccagtggatctgggacttcttcgcccaaggcatctatgacttcgtgcgcgacggcctggtggtcgccaccaaggcgtcgatgtacgccgcgctccagaccctgatcctgctgatcgatgtcagctacaccgccgcccgcgaactgatcgacagccttggcgtgccgcagatgatccgcagcatgtatgccgcgctgccgggtccgattgcggcggggctggccttcttcggcgtgccgcaggcgctgaacatcatcatggtcgcggcggcgacgcgcttctgcatgcgcttcgtgccgttcattgggaggtga

SEQ ID NO: 9 - DNA, 1275 bp, Pseudomonas aeruginosa
gtgtcgatcaagatccaccacggccccaatggctcctacaagacctccggcgcgatccaggatgacgccgtgcccgcgctgaaagacgggcgggtgatcatcaccaacgtgcgcggcttcaccctggagcgggcctatcaggtctttccggacctgcccaacacggcggaaatcatcaacctcgatctggagtcgctggaagacctcgaaaagatgcgcacgtggtttcagtgggcgccccgcggtgccttcctgatcttcgacgaaacccaactgctgtttcccaaatcctggcgggaaaaagacctcgagcgcttcgactaccccggtggaccggaagcggcccacgcggccgaccgccccatgggctggctcgacgcctggacccggcatcggcatttcaactgggacattgtcctcaccacgccgaacatctcctacatccgcgacgatatccgcatgacctgcgagatggcctacaagcattccaacctcgcggtgatcggcatccctggccgctacaaggaggcccagcatgacgcccaactcaaccgtccgcccgccgatggcaccatcatcgagtacaagcggatccgaaagcagaccttcgccctctaccagtccacggccaccggcaagacccaggacaccaaggcgggcaagagcctcttccggtcgcctaagctggttcttctactggcattgctggccggcactattggctttgtctggtatatggggcctctgcgcacgattggcgctccggctgctgcgacacctgccgacgctcctggcgaccctgctcaagcccctgctgcgcccgctgctgtggctgctccaacgcgtcctgctgcgaatagctttcttcctcctgggcttgtacctgatgggcctgctgctgcgcctgttgatctgaacgcccatcccttcgccgatcggcggatctccatccttgcccacgcctaccgcaagtcgcggggcgacatttacatgttcgccctggacgatcccacgggccggcgcctggaactcaccagttggcaactgatcggctccggctaccgggtaacgcccaagggcgagtgcgtcgtagagcttcgctatgaggagtggaaacagaccgtcacctgtaccgggaggcagcccggcgcggtggccagcatcgttccggcagcgcctgttgccgcctccgcagacgcaccggccagggggcagtcgccgctgaccatcgtccccgattccgaatacgcctcgcggccctggaggcagaaatga

SEQ ID NO: 10 - DNA, 552 bp, Pseudomonas aeruginosa
atgaccagaacttcgaacccatgcgcagtggtattggcctttgcggcaattgccgcttcgggaaccgcgatggcggcaaacactatcacattcagcggcgaagtgaccgaccagacctgccaggtcgcggtcaacggcttcaccgaccccacggtgatcctcgacagcgtaccggtgagcgccctcgatggcgcagtgggccgcagcgccggcgaaaccgccttcaccctgcaactcaccgattgcgtcgcgccgacggccgacgagcatttcaccaccctgttccaggccaccaaccccagcgccgccgggaacctggtgaacaccgccgccagcggcgcgacgggcgtggcgctgcaattgctcgatagcgtcggcggcaacccggtcgacctggccggcggcgcggcggtgcccgccggcgacatcgtgctcgccaacggagccaccagcaccagctacgactacgcggtgcagtacgtctccgaagcggcgaccgtgacgcccggtccggtactcggcgtcgtgacctacacgctgcgttacgagtga

SEQ ID NO: 11 - DNA, 450 bp, Pseudomonas aeruginosa
atgaaagctcaaaaaggctttaccttgatcgaactgatgatcgtggttgcgatcatcggtatcctggcggcaattgccattccccagtatcagaactatgttgcgcgttcggaaggtgcttcggcgctggcgacgatcaacccgctgaagaccactgttgaagagtcgctgtcgcgtggaattgctggtagcaaaattaaaattggtactactgcttctactgcgaccgaaacatatgtcggcgtcgagccggatgccaacaagttgggtgtaattgctgtagcaatcgaagatagtggtgcgggtgatattacctttaccttccagactggtacctctagtcccaagaatgctactaaagttatcactctgaaccgtactgcggatggggtctgggcttgtaaatctacccaggatccgatgttcactccgaaaggttgtgataactaa

SEQ ID NO: 12 - DNA, 714 bp, Pseudomonas aeruginosa
atgagcatcgataacgtcagcggcaccagcagcaacaccggcaacgtcaacggcagcaagcgcgccgccggcagcggcgccaccgagaccgggcagagcgtcaagggcagcagcaaccttggcaaggacgaattcctcaagctgctggtggcccagttgaagaaccaggacccgatgtcgccgcagcagaacggcgagttcatcgcccaactggcccagttcagcaccgtggaaggcgtgcagtcgctgaacaagagcatggagagcattctctccaactaccagtcgtcgcaggccctgcaagcctcgtcgctggtggggcgcaaggtcatcgtggcgaccgacaagtcggtggtggacaccaaggacaccttcaaggcgtccctcaacctgccggtctccagcagcaacgtatgggtcaacgtctacgacgacaagggcaccgtggtcaatcgcatcaacctcggccagcaggctgccggcagcgtcagcttcatgtgggacggcaaggacagcagcggcaacatcatgccgccgggcacctacaagttcgaggcgcagacctcgatcgacggcaagacctacgggctccagacctacctgccggccaacgtcgacagcgtcaccctggggcagaacggcggcgagctgatgctcaacctcgcaggcctgggcagcatcgcgctgtccaaagtacagatcatcggccagtaa

SEQ ID NO: 13 - DNA, 747 bp, Pseudomonas aeruginosa
atgcccagacattcgtaccgatccggcaggacatcggcgcttctgctcgtcctgctggcgagcatcgcccaggcgcgggcaagcgtggtggtgaccgggacccgggtgatctatcccggcgaggcacgggagaagaccgtgcaactgagcaatcgcgatgcgtttcccaacgtcgtccaggcctgggtcgacatcgacgccccggacgcgccgccggaccaggcggacgcgccgttcctggtcaacccggcggtattccgcatggccccggacagcggccagacgctgcgcatcgtctacaccgggcagggcctgccgggcgatcgcgaatcgctgttccatctcaacgtcctgcagatcccgccacgcaacagcagccatgcggatcggaaccagatgctgctgatgctgcgcaatcgcctgaagctgttctaccgcccggcgggcatccagggccgtcccgaggacctgcccgggcaactgcgtttcgcactggtccggcgcagcgccggctgggcggtgcgggtggacaacccgagcggctactacgcctcgttcgccagcgccacgctcagcgtcggccagcgccgctggcgcttgcgcagcggcatgctggagccgcgcagccatgccgaatggcaggccgaaacccgagaagccctgccgccgggacgcgtgcgcctgcacgcgctgctgatcaacgactacggcgcccatatggatatccgtcatgacctatcgccatga

SEQ ID NO: 14 - DNA, 1467 bp, Pseudomonas aeruginosa
atggcccttacagtcaacacgaacattgcttccctgaacactcagcgcaacctgaatgcttcttccaacgacctcaacacctcgttgcagcgtctgaccaccggctaccgcatcaacagtgccaaggacgatgctgccggcctgcagatctccaaccgcctgtccaaccagatcagcggtctgaacgttgccacccgcaacgccaacgacggcatctccctggcgcagaccgctgaaggtgccctgcagcagtccaccaatatcctgcagcgtatccgcgacctggccctgcaatccgccaacggctccaacagcgacgccgaccgtgccgccctgcagaaagaagtcgctgcgcaacaggccgaactgacccgtatctccgataccaccaccttcggtggccgcaagctgctcgacggctccttcggcaccaccagcttccaggtcggttccaacgcctacgagaccattgacatcagcctgcagaatgcctctgccagcgccatcggttcttaccaggtcggcagcaacggcgcgggtaccgtcgccagcgtagcgggcaccgcgaccgcttcgggcatcgcctcgggcaccgtcaacctggtcggtggcggtcaggtgaagaacatcgccatcgccgccggcgatagcgccaaggccatcgccgagaagatggacggtgcgatcccgaacctgtcggctcgtgcccgtaccgtgttcaccgctgatgtcagcggcgtgaccggtggttcgctgaacttcgacgtaaccgttggcagcaacaccgtgagcctggcaggcgtgacctccactcaggatctggccgaccaactgaactccaactcgtcgaagctgggcatcactgccagcatcaacgacaagggtgtactgaccatcacctccgctaccggcgagaacgtcaagttcggtgcgcagaccggtaccgctactgccggtcaggtcgcagtgaaggtccagggttccgacggcaagttcgaagcggccgccaagaacgtggtagctgccggtactgccgctaccaccaccatcgtgaccggctacgtgcaactgaactcgccgaccgcctactcggtcagcggtaccggcacccaggcttcgcaggtcttcggcaacgccagcgccgcgcagaagagcagcgttgccagcgtcgacatctccactgccgacggcgcccagaacgccatcgcggtagtcgataacgccctggctgcgatcgacgcccagcgtgctgacctcggtgctgttcagaaccgcttcaagaacactatcgacaacctgaccaacatctcggaaaacgctaccaacgctcgtagccgcatcaaggacaccgacttcgctgccgaaaccgcggcgctgtcgaagaaccaggtgctgcaacaggccggtaccgcgatcctggcccaggccaaccagctgccgcaggcggtcctgagcctgctgcgctaa

SEQ ID NO: 15 - DNA, 1425 bp, Pseudomonas aeruginosa
atggccggtatctcgataggagtaggctcgaccgattacaccgatctggtgaacaagatggtgaacctggagggcgcggctaagaccaaccagctcgccaccctggagaagaccaccactactcgtctcactgccctgggccagttcaagagtgcgatcagcgcgttccagacggcgctcacggcgctgaacagcaatgctgtgttcatggcgcgcaccgccaagtcttccaacgaagacattctcaaggcgagcgccacccagagcgcggtggctggcacctaccagatccaggtcaacagccttgccaccagcagcaagattgccctccaggccatcgccgatccggccaatgccaagttcaacagcggtacgctgaacatctccgtcggcgacaccaagctgccggcgatcaccgtcgactccagcaacaacacgctggccggcatgcgtgacgccatcaatcaggccggcaaggaagcgggcgtcagcgccaccatcattaccgacaactccggctcgcgtctggtgctgagcagcaccaagaccggcgacggcaaggacatcaaggtcgaggtcagcgatgacggcagcggcggcaatacgtcgctcagtcaactggccttcgatcccgcgacggctcccaagctcagcgacggcgccgctgccggctacgtgaccaaggcggcaaacggtgaaatcaccgtcgacggcctcaagcgcagcatcgcctccaacagcgtgagcgacgtcattgacggcgtcagcttcgacgtgaaagccgtcaccgaagccggcaagccgatcaccctgactgtttcccgggacgatgccggggtgaaggacaacgtcaagaagttcgtcgaggcctacaacaccctcaccaagttcatcaacgaacagaccgtggtgaccaaggtcggcgaagacaagaatccggtcactggtgctctgctcggcgatgcctccgtccgtgcgctggtcaacaccatgcgcagcgaactgatcgcctccaacgagaacggcagcgtacgcaaccttgccgccctcggaatcaccacgaccaaggacggtacgctggagatcgacgagaagaagctcgacaaggcgatcagcgccgacttcgaaggcgttgccagctacttcaccggcgataccggcctggccaagcgcctgggcgataagatgaagccctacaccgacgcccaggggattctcgaccagcgcaccacgaccttgcagaagaccctcagcaacgtcgacacgcagaaggctgatctggccaagcggctggcggcgctgcaggagaaactgaccactcagttcaacctgctttccgcgatgcaggatgaaatgaccaagcgtcagaagagcatcacggacaacctcgccagcctgccttacggcagcggcaagaagacctga

SEQ ID NO: 16 - DNA, 1389 bp, Pseudomonas aeruginosa
atgagtttcaacatcggcctgagcggcatccaggcggcctctagcggcctgaacgtcaccggcaacaacatcgccaacgccggcaccgtaggcttcaagcaatcccgcgcggagttcgccgacgtctacgccgcctcggtgctgggttcgggcagcaacccgcagggcagcggcgtgctgctctcggacgtctcgcagatgttcaagcagggcaacatcgactcgaccaacagcgtgctggacctggccatcaacggcaacggcttcttcgtcaccagcaacaacggggcgatcagctacacccgcgccggctacttcaataccgacaagcaggatttcatcgtcgacaacaacggctaccgcctgcagggctatgccgtcgggccgaacggccagttgcagaacggcgtggtcaccgacctcaaggtcgagcgcgccaatcaggcgccgcaggccacctcgagcatccagcagtcgtacaacctcaactcgacgctgaagccgccgaccgtgacgccgttcgatccgtccgacgccgctacctacaactcgtcctcttcgctgggcatctatgacagccagggcaactcccacaccatgagccagttcttcatcaagaacgagccggacccgaatgcgaccccgccgattccggagaacagctggaccatgaaagtgctgatcgacggcgtcaatccgctcgatccgtcgaacaagacgccgatgagcttcaacgtcaccttcgacgccagcggccagatgacctcggttcgggcgccggacggcagcaccagcgggccgggcttcagcatcgacgcgaccaccaacgtgatccagttcagtccggccactggcaatccgccgactcccggcaccggctggattccggcggcctcggacggcaagaccccgccgacctacgcctggaacggcgcgaccggtgccgccagcggcatctccttcgacatgcgcaagaccacccagtactccaccgcgttcgcccagagcaacccgatccaggacggctacaccaccggtcagttggcaggcctggaaatcgacgacaccggggtgatcttcgcccgctacaccaacggccagtccaaggtgcagggccaggtggtgctggccaacttcgccaacatccagggcctgacgccgatcggcaagacctcctgggtgcagtcctcggagtccggcgagccggcggtcggcgcgccgcgctcgggcaccctgggggcgttgcaatccggcgcgctggaagcgtccaacgtggacatctccaacgaactggtgaacctcatcgtccaccagcgcaactaccaggccaatgccaagaccatccagaccgaggatgcggtgacccagaccatcatcaacctgcgctga

SEQ ID NO: 17 - DNA, 237 bp, Pseudomonas aeruginosa
atgtctagagtctgtcaagtaaccggtaagggtccggttaccgggaataacatttcccacgcacacaacaaaacccgccgccgtttcctgccgaacctgcagcaccaccgtttctgggtcgagtccgagaagcgcttcgtacgtctgcgcgtttccgccaagggcatgcgtatcatcgacaagcgtggcatcgaggccgtcctggctgacctgcgtgcccgcggcgaaaaattctaa

SEQ ID NO: 18 - DNA, 351 bp, Pseudomonas aeruginosa
atgaccaacaagatcattcagcagatcgaagctgaacagatgaacaaagagatcccggcgttcgcccccggcgataccgtgatcgtccaggtcaaggtgaaggaaggagaccgtcagcgtctgcaggccttcgaaggcgtcgttatcgccaagcgcaaccgtggcctgaacagcgccttcaccgtgcgcaagatctccaacggcgtgggcgtggaacgtaccttccagacctacagcccgatcgtcgacagcctgagcgtcaagcgtcgcggcgacgtgcgcaaggccaagctgtactacctccgcgccctgtccggcaaggccgcgcgcatcaaggaaaaactggtctaa

SEQ ID NO: 19 - DNA, 603 bp, Pseudomonas aeruginosa
atgcaattgaatgtgaacggcgctcaggctatcgaagtctccgagcgtacctttggtggcgaattcaacgagaccctggtgcaccaggcagtcgtggcctacatggctggcggccgtcagggcagcaaggcgcagaagactcgctccgaagtctctggtggtggcaagaagccgtggcgtcagaagggcaccggtcgtgctcgcgctggcaccatccgtagccccatctggcgtgggggcggcaccactttcgccgctaaaccgcgtagtcacgaacagaagctgaacaagaagatgtaccgcgcagcactgcgctccatccttgccgagctcgttcgtctggaccgtctggtcgtcgttgccgacttcgctgtcgatgcaccgaaaaccaagggcctggttgccaagctggacaccctgggtctgaaagacgtgctgatcgtgaccgatggtgtcgacgagaacctgtacctcgctgcgcgcaacctggcgcacgtcgatgtacgtgatgtccaaggttccgaccccgtcagcctgatcgcctacgacaaggtgctggtcaccgtgtccgccgtgaagaagttcgaggagctgctgggatga

SEQ ID NO: 20 - DNA, 351 bp, Pseudomonas aeruginosa
atgagcgtcaagaaagaaacccgtctgcgtcgcgctcgcaaggcacgcctgaagatgcgcgagctggaaaccgtacgcctctgcgtgtaccgctcttcccagcacatttacgcccaggtcattgcggccgacggcggcaaggtcctggccagcgcctcgaccctggacaaagacctgcgcgaaggtgccaccggcaacatcgatgcagccaagaaagttggtcaactggtcgccgagcgcgccaaagcggccggcgtcacccaggtggcgttcgatcgttctggcttcaagtaccacggccgtgtgaaagcgctggctgatgccgctcgtgaaggcggactggagttctaa

SEQ ID NO: 21 - DNA, 300 bp, Pseudomonas aeruginosa
atgaaccaggaacgcgtattcaaagtgctgcttggtccgcacatctccgagaaagccacgggtctcgcggacggcaagagccaattcgttttcaaggttgccaccgatgcaaccaagctggaaatcaagaaggccgtagaaagcctgttcagcgtgaaggtacagcgcgtcactaccctgaacgtcaagggtaagaccaagcgcaccgctcgcggtctgggcaagcgtaacgactggaaaaaagcgtacatcgctctccagccgggccaagatctcgatttcgccaccagcgctgagtaa

SEQ ID NO: 22 - DNA, 471 bp, Pseudomonas aeruginosa
atgccaagacgtcgtgtagcggccaagcgtgaagtgctggctgatccgaaatacggaagccagatcctggccaagttcatgaaccacgtgatggaaagcggcaagaaagccgttgccgagcgtatcgtttacggtgcgctggacaaggtgaaggaacgcggcaaggccgatcccctggaaaccttcgaaaaagctctcgatgccatcgctccgctggtcgaagtgaagtcccgccgcgtaggtggtgcgacttaccaggttccggtcgaagtccgtccgtcccgtcgtaatgccctggccatgcgttggctggtggacttcgcccgcaagcgtggcgagaagtccatggctctgcgcctggctggcgagctgctggatgccgctgaaggcaagggcgcagcagtcaagaagcgtgaagacgtgcaccgtatggcagaggccaacaaggccttctcgcactaccggttctaa

SEQ ID NO: 23 - DNA, 2523 bp, Pseudomonas aeruginosa
atgacgcaagtcacggtgaaagaactggcccaggtggtcgacacaccggtggagcgcctgctgctgcagatgcgtgacgcaggtctgccgcacaccagtgccgaacaagttgtaacggatagcgagaagcaagccctgctgacccacctgaaaggcagccatggcgatcgcgccagcgagccgcgcaagatcaccctgcaacgcaagaccactaccacgctgaaagtgggtggcagcaagacggtgagcgtcgaagttcgcaagaagaaaacctatgtcaagcgcagccccgacgagatcgaggccgagcgccagcgcgaactcgaggagcagcgcgcggcggaagaggcggagcgtctgaaggccgaagaggccgctgcccgccagcgtgccgaagaggaagcgcgcaaggccgaggaagctgcgcgtgccaaggctgcccaggaagcagcggctactgccggtgccgagcctgccgtggtggcggatgtcgcggttgccgaaccagtggccaagcctgccgccgtggaagagcgcaagaaggaagagccgcgccgcgtgcccaagcgtgacgaggacgatgaccgccgcgaccgcaagcacacccagcaccgtccctcggtcaaggagaaggaaaaggttcctgctccgcgcgtggctccacgcagcaccgacgaggaaagcgatggttaccgtcgcggcggtcgtggtggcaagtcgaagctgaagaagcgcaaccagcacgggttccagaacccgacaggaccgatcgtgcgtgaagtgaacatcggtgagaccatcaccgtagccgagttggccgcccagatgtcggtcaagggtgccgaagtcgtcaagttcatgttcaagatgggctccccggtgaccatcaaccaggtcctggaccaggagaccgcccaactggtcgccgaagagctgggccacaaggtcaagctggtcagcgagaacgcgctggaagaacaactggccgagtccctgaagttcgaaggcgaggcggtcacccgcgcgccggtcgtcaccgtcatgggccacgtcgaccacggcaagacctcgctgctcgactacatccgtcgcgccaaggtcgccgcgggcgaggccggcggcatcacccagcatatcggtgcctaccacgtcgaaaccgagcgtggcatggtgaccttcctggatacccccggccacgccgcgttcaccgcgatgcgtgcccgtggcgcccaggcgaccgacatcgtgatcctggtggtcgcggccgacgacggcgtcatgccgcagacccaggaagccgtgcagcacgcgaaagcggcgggcgtgccgatcgtggtcgcggtgaacaagatcgacaagcccgaggccaacccggacaacatcaagaacggcctggccgctctcgacgtgatcccggaagagtggggcggcgacgctcccttcgtcccggtttcggcgaagctcggtactggcgtcgatgaactgctcgaagcggtcctgctgcaggcggaagtgctcgaactgaaagccactccgtcggccccgggccgtggcgtggtggtcgagtcgcgcctggacaagggacgcggcccggtggccaccgtgctggtccaggacggtaccttgcgccagggcgacatggtgctggtcggcatcaactacggtcgtgtccgcgccatgctcgacgagaacggcaagccgatcaaggaagccggcccgtcgatcccggtcgagatcctcggcctggacggtacgccggatgccggtgacgagatgaccgtggtcgccgacgagaagaaggcccgcgaagtcgccctgttccgtcaaggcaagttccgtgaggtgaaactggcgcgtgcccatgccggcaagctggaaaacatcttcgagaacatgggccaggaagagaagaagaccctgaacatcgtcctcaaggccgatgtgcgcggttcgctcgaggctctgcaaggctcgctcagcggcctgggcaacgacgaagtgcaggtccgcgtggtcggtggcggcgtcggtggcatcaccgagtccgacgccaacctggcgctggcttccaacgcggtgctgttcggcttcaacgtgcgcgccgacgccggcgcgcgcaagatcgtcgaagccgaaggtctggatatgcgttactacaacgtgatctacgacatcatcgaggacgtcaagaaggccctgaccggcatgctcggcagcgacctgcgggagaacatcctgggcatcgccgaggtgcgcgacgtgttccgctcgccgaagttcggcgcgatcgccggctgcatggtcaccgagggcatggtgcaccgcaaccgtccgatccgcgtgctgcgcgacgacgtggtcatcttcgaaggcgagctggagtctctccgtcgcttcaaggacgacgtcgccgaagtgcgtgccggcatggagtgcggcatcggcgtgaagagctacaacgacgtcaaggtcggcgacaagatcgaagtgttcgagaaggtcgaagtcgcacgcagcctttga

SEQ ID NO: 24 - DNA, 213 bp, Pseudomonas aeruginosa
atgagaagacttaagcgtgatccgttggaaagagccttcttgcgtggttatcagaacggcataaccggtaaatctcgtgatctttgtccgttcacccatcctacgacgcggcagtcctggctcaacggctggcgcgagggccgtggcgacaactgggacggcctcactggcacggccggcttacaacgtctcaatcaactccagcacgtgtaa

SEQ ID NO: 25 - DNA, 2277 bp, Pseudomonas aeruginosa
atgttgaatcgagagctcgaagtcaccctcaatctcgccttcaaggaggcgagggccaaacgccatgaattcatgacggttgagcacctgctgctggccttgctggacaatgaggcggcggcaacggtattgcgtgcgtgcggtgccaacctggacaagctgcggcgcgacctgcaggaattcatcgattccaccacgccgctgatcccgcagcacgacgacgagcgcgaaacccagccgacgctgggcttccagcgcgtcctgcagcgtgcggtgttccacgtgcagagttccggcaagcgcgaagtgaccggggccaatgtcctggtggcgatcttcagcgaacaggaaagccaggcggtgttcctgctcaagcagcagagcatcgcgcgtatcgatgtggtgaactacatcgcccatggtatctccaaggtgcccgggcatgccgaacatccgcaggatggggagcaggatatgcaggatgaggaaggtggcgagtcggccacgtccaaccatccgctggacgcctatgccagcaacctcaacgaactggctcgccaggggcggatcgacccgctggtggggcgtgagcatgaagtcgagcgggtggcgcagatccttgcccgccggcgcaagaacaacccgctgctggtaggcgaggcgggggtcggcaagacggccatcgccgagggcctggccaaacgcatcgtcgatggccaggtgccggacctgctggccgacagcgtggtctactccctcgacctgggtgccttgctcgcgggtaccaagtaccgcggcgacttcgagaagcgcttcaaggccttgctcaacgagttgcgcaagcgcccgcacgcggtgctgttcatcgacgagatccataccatcatcggcgccggtgcggcatccggcggggtaatggacgcctccaacctgctcaagccggttctgtcctcgggcgagatccgctgcatcggctcgaccaccttccaggagttccgcggcatcttcgagaaggaccgggccttggcgcggcgcttccagaaggtcgacgtgaccgagccgtcggtggaagacacctatggcatcctcaagggcctcaaggggcgcttcgagcagcatcaccacatcgagtacagcgacgaggcgctgcgcgccgcggccgagctggcggcgcgctacatcaacgaccggcacatgccggacaaggccatcgacgtcatcgacgaggcgggcgcctaccagcgcctgcagccggaagagaagcgcgtgaagcgcatcgaggtggcgcaggtcgaggatatcgtggcgaagatcgcgcggatcccgccgaaacacgtcaccacctccgacaaggagttgctgcgcaacctcgaacgcgacctcaagctgaccgtgttcggccaggacgacgccatcgagtcgctgtccaccgcgatcaagctgtcccgggccgggctaaaggcgccggacaagccggtcggctcgttcctcttcgccggtcccaccggcgtgggcaagaccgaggtggcgcggcagttggcgaaggccttgggcgtggagctggtgcgcttcgacatgtccgagtacatggagcggcataccgtgtcgcggctgatcggtgcgcctccgggctacgtcggcttcgaccagggcggcctgctcaccgaggcgatcaccaagaccccgcactgcgtgttgctgctcgacgagatcgagaaggctcacccggaggtcttcaacctgctgctgcaggtgatggaccacggcaccctgaccgacaacaacgggcgcaaggcggacttccgcaacatcatcctgatcatgaccaccaacgccggcgcggaagtggcggcgcgcgcgtcgatcggcttcaaccagcaggatcacaccaccgatgcgatggaagtgatcaagaagagcttcaccccggagttccgcaaccgcctggataccatcatccagttcggccgcctgagcaccgagacgatcaagagcgtggtcgacaagttcctcaccgagctgcaggcgcagctggaggacaagcgcgtccagctcgaggtcagcgatgcggcgcgcggctggctggcggagaagggctacgacgtgcagatgggggcgcgaccgatggcgcggcttatccaggacaagatcaagcggccgttggccgaggagatcctgttcggcgagctggccgagcatggcggcctggtgcatgtcgacctgaagggcgacgagctggccttcgagttcgagatcacggcggcggagcccgcctga

SEQ ID NO: 26 - DNA, 306 bp, Pseudomonas aeruginosa
atgatccccggtgaatacgacatccagcccggcgatatcgaactcaacgccggccgccgcaccctcgccctgagcgtggcgaacaccggtgaccggccgatccaggtcggctcgcactaccacttcttcgaggtcaacgacgccctcgccttcgaccgtccggccacccgtggcatgcgcctgaacatcgccgccggcaccgcggtgcgcttcgaaccggggcagagccgcgaggtggagctggtggaaatcggcggcggacggcgggtatacggctttgccgggcgggtgatgggggacctctag

SEQ ID NO: 27 - DNA, 252 bp, Pseudomonas aeruginosa
atgtccctgaaaatcactgacgattgcatcaattgcgacgtctgcgaacccgaatgcccgaacggcgcgatctcgcaaggcgaagaaatctatgtgatcgatcccaatctctgcaccgagtgcgtcggccactacgacgagccgcagtgccagcaggtctgtccggtggactgcattccccttgacgacgccaatgtcgagagcaaggaccagttgatggagaaataccggaagatcaccggcaaggcctga

SEQ ID NO: 28 - DNA, 654 bp, Pseudomonas aeruginosa
atgggactcgagctgggcttccgtgagctcggcgaggttccctacgaaccgacctggcatgccatgcagcgcttcgttgccgagcgcgacaaatcggtaatggacgaagcctggctgttgcagcaccctgcggtcttcacccaggggcaggccggcaaggcggagcatgtgctgttccccggcgacatcccggtgatccaggtagatcgcggcggccaggtgacctaccacggccccggccagttggtcacctacctgctgctcgatgtccgccgcctgggactgggcgtgcgcgagctggtcagccggatcgagcagagcctgatcggcctgctggcgtcctatgacgtgcaggccgtggccaagccggacgcgccgggtgtctacgtcgacggggcgaagatcgcctccctcggcctgcgcatccgcaacggctgttccttccatggcctggcgctgaacctggacatggacctgcgaccgttccaacgaatcaatccctgcggctacgccgggatgcccatgacccaactgcgcgacctggttgggccggtggattttgccgaggtgtgtacccgattgcgcgctgagctcgtctcacgccttggctacgctgaacagaagacccttacgggcgggatcgaactgacatga

SEQ ID NO: 29 - DNA, 1539 bp, Pseudomonas aeruginosa
atgagtcaagcgcacaccccctccgctccactcgccgaagtctacgatgtcgccgtggtcggcggtggcatcaacggtgtagggatcgccgccgacgcagccgggcgcggcctgtccgtgttcctttgcgaacagcacgacctggcccagcacacttcctcggccagcagcaagctgatccacggcggcctgcgctacctcgaacactacgaattccgcctggtccgcgaagccctggccgagcgcgaggtcctgctggccaaggcgccgcacatcgtcaagccgctgcgcttcgtcctgccgcaccgtccgcacctgcgcccggcctggatgatccgcgccggcctgttcctctacgatcacctcggcaagcgcgagaagctgcccgcctcgcgcggcctgcgcttcaccggcagcagcccgctgaaggccgagatccgccgtggcttcgagtactccgactgcgcggtggacgatgcgcgcctggtggtgctcaacgcgatctccgcccgcgagcacggcgcccacgtccatacccgcacccgctgcgtcagcgcccgtcgcagcaagggactctggcacctgcacctggagcgcagcgacggcagcctgtattcgatccgcgcccgcgccctggtgaacgccgccggcccctgggtagcgcgcttcatccaggacgacctgaaacagaagtcgccctacggtatccgcctgatccagggcagccacatcatcgtgccgaagctgtacgaaggcgagcacgcctacatcctgcagaacgaagaccgccgcatcgtcttcgccatcccctatctggaccgtttcaccatgatcggcaccaccgatcgcgagtaccagggcgacccggccaaggtggcgatcagcgaagaggaaaccgcctacctgctgcaagtggtgaacgcccatttcaagcagcaactcgcggccgccgacatcctgcacagcttcgccggcgtgcgtccgctgtgcgacgacgagtccgacgagccttcggcgatcacccgcgactacaccctgtcgctctccgccggcaacggcgagccgccgctgctctcggtgttcggcggcaagctgaccacctaccgcaagctggccgaatcggcgctgacgcagctccagccgttcttcgccaatctcggcccggcctggaccgccaaggcaccgctgccgggcggcgagcagatgcagagcgtcgaggcgctcaccgagcaactggccaaccgctacgcctggctcgaccgcgaactggcgctgcgctgggcgcgcacctacggcacccgggtgtggcgcctgctggacggggtcaatggcgaagccgatctcggcgagcacctgggcggcggcctgtatgcccgcgaagtggactacctgtgcaagcacgagtgggcccaggacgccgaggacatcctctggcgccgcagcaagctcggcctgttcctctcgccgagccagcaggtgcgcctcggccagtacctgcagagcgagcatccgcatcgcccgcgggtgcatgcggcctga

SEQ ID NO: 30 - DNA, 888 bp, Pseudomonas aeruginosa
atggcaagccacgaacactattacgtccccgcccagagcaagtggccgatcatcgccagcatcggcctgctggtgacggtgttcggcctgggtacctggttcaacgacctcaccgccggccacaaggaatcccatggcccgtggatcttcttcgtcggcgggctgatcatcgcctacatgctgttcggctggttcggcaacgtcatccgcgagagtcgcgccggactctacagcgcgcagatggaccgctcgttccgctggggcatgagctggttcatcttctccgaggtgatgttcttcgccgccttcttcggcgcgctgttctacgtccgccacttcgccggtccctggctcggcggcgagggtgccaagggcgtggcgcacatgctctggccgaacttccagtacagctggccgctgctgcagaccccggacccgaagctgttcccgccgccgagcgcggtgatcgagccgtggaagctgccgctgatcaacaccatcctgctggtcacctccagcttcaccgtgaccttcgcccaccacgccctgaagaagaacaagcgcggcccgctcaaggcctggctggcgctgaccgtgctgctggggatcgccttcctgatcctgcaggccgaggagtacgtgcacgcctacaacgagctgggcctgaccctcggcgccggcatctacggctcgaccttcttcatgctcaccggcttccacggcgcccacgtgaccctcggcgcgctgatcctcggcatcatgctgatccgcatcctgcgcggtcacttcgatgccgagcaccacttcggcttcgaggccgccagttggtactggcacttcgtcgacgtggtctggatcggcctgttcatcttcgtctacgtgatctga

SEQ ID NO: 31 - DNA, 1125 bp, Pseudomonas aeruginosa
atgctgcgacatccacgagtctggatgggcttccttttgctctcggcgatcagccaggcgaatgccgcctggacggtcaacatggccccgggcgctaccgaggtgagccggtccgtcttcgacctgcacatgaccatcttctggatctgcgtggtgatcggcgtgctggtcttcggcgcgatgttctggtcgatgatcgtccaccgccgctccaccggccagcagccggcgcacttccacgagagcaccacggtggaaatcctctggacggtggtgccgttcgtgatcctggtggtgatggcggtgcccgccacccgcaccctgatccacatctacgacacttccgagccggagctggacgtgcaggtcaccggctaccagtggaagtggcagtacaagtacctgggccaggacgtcgagtacttcagcaacctggccaccccgcaggatcagatccacaaccggcaggcgaaggacgagcattacctgctggaggtggacgagccgctggtgctgccggtgggcaccaaggtgcgcttcctgatcacctccagcgacgtgatccattcctggtgggtgccggccttcgcggtcaagcgcgacgccatccccggcttcgtcaacgaggcctggaccaaggtcgacgagcccggcatctatcgcggccagtgcgccgagctgtgcggcaaggaccacggcttcatgccgatcgtggtcgacgtcaagcccaaggccgagttcgaccagtggctggccaagcgcaaggaagaggcggcgaaggtcaaggaactgaccagcaaggagtggaccaaggaagagttggttgcgcgcggcgacaaggtctaccacaccatctgcgccgcctgccaccaggccgaaggccagggcatgccgccgatgttcccggcgctgaagggttcgaagatcgtcaccgggcccaaggagcaccacctggaagtggtcttcaacggcgtgcccggcaccgccatggcggccttcggcaagcagctcaacgaggtcgacctggccgcggtgatcacctacgagcgcaacgcctggggcaacgacgatggcgacatggtcaccccgaaagacgtcgtcgcctacaagcagaaacaacaatag

SEQ ID NO: 32 - DNA, 1593 bp, Pseudomonas aeruginosa
atgagtgctgtgatcgatacgcccgaccaccatgccggcgaccaccaccacggaccggccaagggcctgatgcgctgggtgctgacgaccaaccacaaggacatcggcaccctctacctgtggttcagcttcatgatgttcctgctcggcggctcgatggccatggtgatccgcgccgagctgttccagcccggcctgcagatcgtcgagccggcgttcttcaaccagatgaccaccatgcacggcctgatcatggtcttcggcgcggtgatgccggccttcgtcggcctggccaactggatgatcccgctgatgatcggcgcgccggacatggccctgccgcggatgaacaacttcagcttctggctgctgccggcggccttcggcctgctggtcagcaccttgttcatgcccggcggcggccccaacttcggctggaccttctatgcgccgctgtcgaccaccttcgccccgcacagcgtgaccttcttcatcttcgccatccacctggccgggatcagctcgatcatgggcgcgatcaacgtgatcgccaccatcctcaacctgcgcgccccgggcatgaccctgatgaagatgccgctgttcgtctggacctggctgatcaccgcgttccttttgatcgcggtgatgcctgtgctggccggcgtggtgaccatgatgctgatggacatccacttcggcaccagcttcttcagcgccgccggcggcggcgacccggtgctgttccagcacgtgttctggttcttcggccaccccgaggtgtacatcatgatcctgcccgccttcggtgcggtcagtgcgatcatcccgaccttcgcgcgcaagccgctgttcggctacacctcgatggtctacgccaccgccagcatcgccttcctctccttcgtggtctgggcgcaccacatgttcgtggtcggcatcccggtcaccggcgagctgttcttcatgtacgccaccatgctgatcgcggtgcccaccggggtgaaggtgttcaactgggtgaccaccatgtgggagggttcgctgaccttcgagacgccgatgctgttcgccgtggccttcgtcatcctgttcaccatcggcggcttctccggactgatgctggcgatcgccccggcagacttccagtaccacgacacctacttcgtggtcgcccacttccactacgtgctggtgcccggcgcgatcttcggcatcttcgcctcggcttactactggctgccgaagtggaccggccacatgtacgacgagaccctcggcaagctgcacttctggatgagcttcatcggcatgaacctggcgttcttcccgatgcacttcgtcggcctcgccggcatgccgcgacggatccccgactacaacctgcagttcgccgacttcaacatggtctcgtcgatcggcgccttcatgttcggcaccacccagctgctgttcctgttcatcgtcatcaagtgcatccgcggcggcaagccggcccctgccaagccctgggacggcgccgagggcctggagtggagcatcccctcgccggcgccctaccacaccttcagcaccccgcccgaggtcaagtga

SEQ ID NO: 33 - DNA, 1026 bp, Pseudomonas aeruginosa
atgttcgacatgatggacgcggcccggctcgagggtctccacctcgcccaagacccggccacgggactcaaggccattatcgccatccacagcacgcgactcggcccggcgctgggtggttgtcgctacctgccttaccccaacgacgaagccgccatcggcgacgccatccgcctggcccagggcatgagctacaaggcggccctggccgggctggagcagggcggcggcaaggcggtgatcatccgcccgccgcacctggacaatcgcggcgcgctgttcgaggccttcgggcgcttcatcgaaagcctcggcggacgctacatcactgcggtggacagcggtacctccagcgccgacatggactgcatcgcccagcagacccgccacgtcaccagcaccacccaggccggcgacccctcgccgcataccgccctcggcgtgttcgccgggattcgcgccagcgcccaggcgcgcctcggcagcgacgacctggaaggcctgcgggtcgcggtgcaggggctcggccacgtcggctacgcattggccgagcaactggcggcggtcggcgccgagctgctggtctgcgacctcgatcccggccgggtgcaactggccgtcgagcagctcggtgcccatccgctggcgccggaggcattgctctccaccccttgcgacatcctcgcgccctgcggcctgggcggcgtgctcaccagccagagcgtcagccagttgcgctgcgcggcggtggccggggcggcgaacaaccagttggagcggccggaggtcgccgacgagctggaggcgcgcggcatcctctatgcgccggactacgtgatcaactccggcggcctgatctacgtcgccctcaagcaccgcggcgccgatccgcacagcatcaccgcgcacctggcgcggattcccgcgcggctcaccgagatctatgcccatgcccaggccgaccaccagtcgccggcgcggatcgccgaccgtctggcggaacggattctctacggcccgcagtga

SEQ ID NO: 34 - DNA, 426 bp, Pseudomonas aeruginosa
atgttcggaatcagcttcagcgaactgttgctggtcgggctggtcgccctgctggtgctcggccccgagcgcctgccgggcgccgcacgtaccgccggcctgtggatcggccgcctgaagcgcagtttcaataccatcaagcaggaagtggaacgggaaatcggcgcggacgagattcgccggcaactgcacaacgagcacatcctctcgatggagcgcgaagcgcagaagctgctggccccgctgaccggccagaatcccccgcaggaaaccccgccgccggcggccgagagcccggcgccgagcgtacccacgccgccgccgaccagcacgcctgcggttccgcccgcggacgccgcggcaccgccggcagtcgctgcctccactcccccttcgccaccgtccgagacgccgcgtaatccatga

SEQ ID NO: 35 - DNA, 1044 bp, Pseudomonas aeruginosa
ttgaagcaacagttcgaacgctcgccttccgagagttatttctggcccgtcgtcctggcggtggtcctgcacgttctgatcttcgccatgctgttcgtcagctgggcgtttgctccggagcttcctccctccaagccgatcgtgcaggccacgctctaccagctcaagtcgaagagccaggcgacgacacagaccaaccagaagatcgctggcgaggcgaagaagaccgcctccaagcaatacgaagtcgagcagctcgaacagaagaagctcgagcagcagaaactcgagcaacaaaagctggaacagcagcaggtcgctgctgcgaaagcggcggaacaaaagaaggctgacgaggctcgaaaggccgaggcccagaaagccgccgaggcgaaaaaggccgatgaagccaagaaagctgccgaggccaaggccgccgaacagaagaagcaggctgacatagccaagaagcgcgccgaggacgaggccaagaaaaaggccgctgaggacgccaagaaaaaggcagccgaggacgcgaagaagaaagcggccgaggaggccaagaagaaggctgctgcggaagcggcgaagaagaaagccgccgtcgaggccgccaagaaaaaggccgccgccgctgccgcggcagcccgcaaggctgccgaggacaagaaggcgcgggcattggccgagttgctttcggatacgaccgagcgccagcaggccctggccgacgaggtgggcagcgaggtcaccggcagtctcgacgacctgatcgtcaacctggtgagccagcagtggcggcgtcctccatcggcgcgtaatggaatgagcgtagaagtactgatcgaaatgctgccggacggtaccatcaccaatgccagcgtcagccgttcgagtggcgacaagcctttcgacagttcggcggtggcggcggtgcgcaacgtcggccgtattcccgagatgcagcaattgccgcgggctaccttcgacagcctgtatcgtcagcgccgcatcatctttaaaccggaggatttgagtctgtga

SEQ ID NO: 36 - DNA, 249 bp, Pseudomonas aeruginosa
atgggcatttttgactggaaacactggatcgtcatcctgatcgtcgtggtactggtgttcggcaccaagcgcctgaagaacctcggttccgacgtcggcgaagcgatcaagggcttccgcaaggcggtgaacaccgaggaagacgacaagaaggaccagcccgccgcccagccggcccaaccgctgaaccagccgcacaccatcgacgcccaggcgcagaaggtcgaagagccggcgcgcaaggactga

SEQ ID NO: 37 - DNA, 531 bp, Pseudomonas aeruginosa
atgcaaaacgccaagctcatgctgacctgtctcgctttcgcggggcttgccgcactcgccggttgctcgtttcctggcgtctataaaatcgacatccagcaaggcaacgtcgtaacgcaggacatgatagaccagttgcgtcccggaatgacccgacgccaagtgcggtttatcatgggcaacccgctcatcgtcgataccttccacgccaatcgttgggactacctctacagcatccagccgggcggcggtcgtcgccagcaggagcgcgtcagcctgttcttcaacgacagcgaccagctggccggcctgaacggcgacttcatgcccggcgtcagccgcgacgaagcgatcctcggcaaggaaggcagcaccaccgtcacccagccggccgaccagcagaaaccggaagcgcagaaagaagagccgccgaaaccgggctccaccctggaacagctccagcgcgaagtggacgaagcccagccggtaccggttccgactcccgaacctctggaccccagcccgcaatga

SEQ ID NO: 38 - DNA, 1266 bp, Pseudomonas aeruginosa
atgagcatgaccccgatcgcccgtgccgtggcttttgccgcgctgggttccagcattaccgtcccaaccctggcccatgccgagttcatcaaggacagcaaggccagcatcgaactccgcaacttctacttcaaccgtgatttccgccaggaaggcgccagccagtccaaggccgaggaatgggcccagggcttcctcctgcgctacgagtccggctacaccgagggcaccatcggtttcggcgtcgacgccatcggcctgctcggggtgaagctcgactccagccccgaccgcagcggcaccggcctgctcaagcgcgaccgcgagaccggccgcgcccaggacgactatggcgaggcagggatcaccgccaagctgcgggcgtcgaagagcaccctgaagatcggcaccctgacccccaagctgccggtgatcatgcccaacgacagccgcctgctgccacagaccttccagggcggcgcgctgaactccatggagatcgacggcctgaccctcgacgccgggcgcctgaagaaggtcaaccagcgcgactcctcggacaacgaggacatgaccatcaccggtggcggcaagcgcaacatcgtggtccgttccgggctgacttcggacaagttcgacttcgccggcggcagctacaagtggaccgacaacctcagcaccagctaccactacggcaagctcgacaacttctacaagcagcattacctcggcctggtgcacaccctgccgatcgccgacaagcagtcgctgaaatccgacatccgctgggcgcgctccaccgacgacggttccagcaacgtcgacaacaaggcgctcaacgccatgttcacctacagcctcggctaccacgccttcggcgtcggctaccagaagatgagcggcgacaccggcttcgcctacatcaacggcgccgacccctacctggtgaacttcatccagatcggcgacttcgccaacaaggacgagaaatcctggcaggcgcgctacgactacaacttcgccggcgtcggcattcccggcctgaccttcatgacccgctacgtcaagggcgacaacatcgacctgctgaccacctcgggcgaaggcaaggaatgggaacgcgacatggacatcgcctacgtgttccagagcggcccgttgaagaacctcggggtgaagtggcgcaacgcgaccatgcgcaccaactacaccaacgactacgacgaaaatcgcctgatcgtcagctacacgctgccgctctggtaa

SEQ ID NO: 39 - DNA, 438 bp, Pseudomonas aeruginosa
atggatttaacgagcaaggtcaaccgactgcttgccgagttcgcaggccgtatcggtttgccttccctgtccctcgacgaggagggcatggcgagcctcctgttcgacgaacaggtgggcgtcaccctgttgctgctcgccgagcgcgagcgtctgttgctggaggccgatgtggcgggcatcgatgtgctgggcgaggggatctttcgccagctcgccagcttcaaccgccattggcaccgtttcgatctgcatttcggcttcgacgagctgaccggcaaggtccagttgtatgcgcagattctcgcagcgcaactgaccctcgaatgcttcgaggcgaccttggccaatctgctcgatcacgccgagttctggcagcgcctgctgccgtgcgacagtgatcgcgaggcggtcgctgcggtcggcatgagggtttga

SEQ ID NO: 40 - DNA, 1656 bp, Pseudomonas aeruginosa
atgatcgctcgtctccttgccgcgctcggccttgcggccttcgctccggccctctgggccgacgcgctgaccggcgacgtgcagcgccagccgctgaacgtctcggccatcgtcatgttcgtcgccttcgtcggcgcgaccctctgcatcacctactgggcatccaagcgcaaccgctcggcggccgactactatgccgcaggcggccgcatcaccggcttccagaatggcctggcgatcgccggcgactacatgtcggcggcatccttcctgggtatttcggcgctggtcttcacttccggctacgacggcctgatctactcgatcggcttcctggtcggctggccgatcatcctcttcctcatcgccgagcgcctgcgcaacctgggcaagtacaccttcgccgacgtcgcctcctaccgcctcaagcagaagcagatccgcaccctgtcggcctgcggctcgctggtggtggtggcgttctacctgatcgcgcagatggtcggcgccggcaagctgatcgagctgctgttcggcctcaactaccatgtcgcggtggtcctggtgggcatcctgatggtgctctacgtgctgttcggcggcatgctggcgaccacctgggtgcagatcatcaaggccgtgctgttgctctccggcgcctcgttcatggcgatcatggtgctcaagcacgtcaacttcgacgtcagcacgctgttctccgaagccatcaaggtgcatccgaagggcgaggcgatcatgagtccgggcggcctggtcaaggatccgatctcggcattctccctgggcttcgcgctgatgttcggcaccgccgggctgccgcacatcctgatgcgcttcttcaccgtcagcgacgccaaggaagcgcgcaagagcgtgttctacgccaccggtttcatcggctacttctacatcctgaccttcatcatcggtttcggcgcgatcctgctggtcagcaccaacccggacttcaaggacgccaccggcgccctgatcggcggcaacaacatggcggcggtgcacctggccgacgcggtgggcggcagcctgttcctcggcttcatctcggcggtggccttcgccaccatcctcgcggtggtcgccgggctgaccctggccggggcctcggccgtctcccatgacctgtacgccagcgtgttcaagggcggcaaggccaacgagaaggacgagctgcgggtctcgaagatgaccaccgtggccctgggcgtggtcgccatcgtgctcggcatcctcttcgagaagcagaacatcgccttcatggtcggcctggccttctccatcgccgccagctgcaacttcccggtgctgctgctctccatgtactggaagaagctgaccacccgcggcgccatgatcggcggctggatgggcctgatcaccgcggtgggcctgatggtcctcggcccgaccatctgggtgcagatcctcggccacgagaaagccatctacccgtacgagtacccggcgctgttctcgatgatcgtcgccttcgtcggtatctggttcttctccatcaccgacaagtcggcggcggccgacgaggagcgggcgcgcttcttcccgcagttcatccgttcccagaccggcctgggcgcctctggcgcggtggcccactaa

SEQ ID NO: 41 - DNA, 918 bp, Pseudomonas aeruginosa
gtgaattatcccgtggatcacctcaccgctctcaaggtcttccgcgccgtggccgcgaatggcggcttcgccgccgcggcccggcagatgaatctctcgccggccgccgtgagcaagaacgtcgccgagctggaagcgcacctcaaggtgcgcctgatcaatcgcaccacccgcagcatgagcctgaccgaggccggcgaagtctaccggcagcgcctggagcgcatcctcgacgacctcgaggccgccgacgccgcgctcacttcgatgcagcagggccccagcggcctgctgcgggtcagcgccccgctgaccctcgccctcacctgcctgaccccggccattccggcttttctccagcgttatccggagctgcgcctggaactgctcctgcaggacggccgccaggacctgatcgccgaaggcatcgacctggccctgcgtggtagcgaccgggttgcggactccggcctggtcgcccgcccgctgctggtcctggagcacgtgctctgcgcggcgccggcctacctcagtcagcatggccagccgctgcggccggaggccctgcgcgaacacgagtgcatccgcttcagcctctctggccatgccgaccgctggaccttccgcaaggaccgcgagtgcatagcggtgcctatcgccggccgttaccgggtgtcctccagcctggcggtgcgcgatgccctgctcgccggcttcggcttgagcctgatcccacggctgtacgtacaggcggaactggccgaaggccgcctggttgaattgctggctgactggaaggccgacgagacggcgatccacgcggtctatccatcgcgccaactggccggcaagacccgggtgtttctcgacttcctgaccgagacaatggcgcagggccatgattccccaacgctgtaa

SEQ ID NO: 42 - DNA, 855 bp, Pseudomonas aeruginosa
atgaccacttctttgcaacctgtacatgccttggttccaggcgcaaacctggaagcctacgtgcactcggtgaacagcattccgctgttgtcgccggaacaggagcgtgaactggccgaacgcctcttctatcagcaagacctggaagcggcgcggcagatggtgttggcgcacctgcgcttcgttgttcatatcgccaagagttattcggggtacggcctggcccaggccgacctgatccaggaaggcaacgtcggcctgatgaaggccgtgaagcgtttcaacccggaaatgggcgttcgcctggtgtccttcgccgtgcactggatcaaggcggagatccacgagttcatcctgcgcaactggcggatcgtcaaggtcgccaccaccaaggcgcagcgcaagctgttcttcaacctgcgcagccagaagaagcgcctggcctggttgaacaacgaggaagtccatcgcgtcgccgagagcctgggggtcgagccccgcgaagtgcgcgagatggaaagccgcctgaccggccaggacatggcgttcgacccggccgccgacgcggacgacgagagcgcctaccagtcgccggcgcattacctcgaagaccaccgctacgacccggcgcgccagttggaagacgccgactggagcgacagctccagcgccaacctgcacgaggcgctggaaggcctcgacgagcgcagccgcgacattctccagcagcgctggctgtccgaggagaaggccacgctgcacgacctggcggaaaagtacaacgtttccgccgagcgtatccggcagctggaaaagaacgccatgagcaagctgaaagggcggattctcgcctga

SEQ ID NO: 43 - DNA, 1005 bp, Pseudomonas aeruginosa
atggcactcaaaaaagaagggccggagtttgaccacgatgatgaagtgctcctcctggagcccggcatcatgctggacgagtcgtctgccgacgagcagccttctccccgggcaactccaaaagccaccacttccttctcttccaaacaacacaagcacatcgactacacgcgcgcgttggacgcaacgcagctgtatctcaacgaaatcggtttctcgcccctgttgacgcccgaagaggaagtccacttcgctcgtctggcgcagaagggcgatcccgctggtcggaagcggatgatcgagagcaacctgcggttggtggtgaagatcgcccggcgctatgtcaatcgcggactgtccctgctcgacctgatcgaggaaggcaacctaggcctgatccgcgccgtggagaagttcgatccggagcgcggattccggttctcgacctacgccacctggtggatccgccagaccatcgagcgggccatcatgaaccagacccggaccattcgcttgccgatccatgtggtcaaggagctcaacgtctacctgcgtgcggcgcgggaactgacccacaagctcgaccacgaaccttcacccgaagaaatcgccaacctgctggagaagccggtcgccgaggtcaagcgcatgctcggcctgaacgaacgggtgacttcggtagacgtctctcttggtccggactcggacaagaccctgctggatacgctcaccgacgatcgccccaccgatccgtgcgagctgctgcaggatgacgatctcagcgaaagcatcgaccagtggctgacggaactcaccgacaagcagcgtgaggtggtgattcgccgcttcggcttgcgcggtcacgaaagcagcacgctggaagaggtcggccaggaaatcggcctgacccgcgagcgggttcgtcagatccaggtcgaggcgctgaagcgcctgcgggagattctggagaagaatggcctgtcgagtgacgcgctgttccagtga

SEQ ID NO: 44 - DNA, 828 bp, Pseudomonas aeruginosa
atggacaaaccggcctcgcggcatttcagcgtcttgatcatcgatgatgaaccccaggtgacctcggaactccgcgaactgctggaaaacagtggctaccgttgcgtaaccagcacccaccgggagtcggcgatcgccagcttccaggccgacccgaacatcggcctggtcatctgcgacctctacctgggccaggacaacggtatccgcctgatcgagagcctcaaggaagtcgccggcaacggcaggttcttcgaatcgatcatcctcaccggtcacgatggccgccaggaagtgatcgaggccatgcgggtcggcgccgccgactactaccagaaaccggtggcgccgcaggaactgctgcatggcctcgaacgcctggagagccgcctgcacgagcgcgtccgcagccagttgagcctgagccacgtcaaccagcgcctggaatacctcgccgaatcgctgaactcgatctaccgcgacatccacaagatcaagtacgaggtacacggcaacagccagccgagcgccctcaggagcgaagacagccagccgtccgcgccgccggcgccggtcgcggaaagccaggtgtccccgagcaatccgctgttcggcaagctgtcgccccgccagcaggcggtggcgcggctggtgagcaagggcctgaccaactaccagatagcctacgagctgggcatcaccgagaacacggtgaagctgtacgtctcccaggtgctgcgcctgatgcatatgcacaaccgcacccagttggcgctggccctgtcgcctgcggcgatgcagcagggcagcggagcggtggtgcactga

SEQ ID NO: 45 - DNA, 639 bp, Pseudomonas aeruginosa
atgccatcggtcagcgatcccagggcgcttgcccgtcttctggaattgatactgtccaccgcccaggccggtctcgccttctccaaggaccgcttcgacatcggacgctttcgcgcgctccagcatgccgtcgccgaattcatcgccagcgaccagggggtagcgtacgaacgggtcgagaactggatcgctctggacagccactacccgaccccgaaactggacgtccgtgcgttgatcctcgactcgcaacagcgggttctgctggttcgggaggcctcggatagtcgctggaccctaccaggcggctggtgcgacgtaaacgagtcgccggcagatgcggtagttcgcgaaacccaggaagaaagcggactggaagttcgagcgatacgcctgttggcattgctggacaagcacaagcatccccaccctccccaactgccgcatgcgttgaaagccttcttcttctgccacgtcaccggtggcagtcttcagcaacaaacggacgaaacgtcggcggccgagtacttcacggtggacgccttgccgccgctttccgagcaccgggtactggcctcgcagatccagacgttatggcagcgaatccacgcggaaacgccagaggcgcttttcgactag

SEQ ID NO: 46 - DNA, 216 bp, Pseudomonas aeruginosa
atgccagccgtcaaagtaaaagagaacgaacccttcgacgtagccctgcgtcgtttcaagcgctcctgcgaaaaagcaggtgtactggctgaagttcgcagccgcgagttctacgagaagcccactgccgagcgcaagcgcaaggccgctgccgcagtgaagcgccacgcgaagaaagtacagcgcgaacagcgccgtcgcgagcgcctgtactga

SEQ ID NO: 47 - DNA, 537 bp, Pseudomonas aeruginosa
atgttgaatgggccgattccacctcatatcgatccgcgcaagttggtcgaccgcggcgccaccctcgaaggtgtgtacgcgctcgccgatatgccgcgtgtatgcgagcaactgaccagcgatgccggtgaggtgcgcgtgaaggtttccttcgagcgcgaccaccagaagctggcggtcatgcatatgcagctcgataccgaggtgagcatggtatgccaacgctgcctggatgcggcggccattcccgtgcatggcgaatatacctacgccattcttcgggaaggtcagtccgccgacggtctgccgaaggggtacgatgcgctggaagtgggggaggaacccctggacctgctggcgctggtcgaggacgagctgttgctcgccttacccatcgttccggcccatgaccccgaagtatgtcagcacccggctggattcgtcgtcgaagacgagccggagtcgagcgaagtcgaggacaagcgtcctaatccgttcagcgtactggcacagttgaagcgtgacccaaacgtttag

SEQ ID NO: 48 - DNA, 477 bp, Pseudomonas aeruginosa
atgcgcacatccttcttcgctgccgcggccctgctggccgtttccgctttcgcccaggcccacgagtacaacgccggccaactgcacatcgagcatccttgggcgctggccctgccgccgacctcgccgaacggcgcggcctacttcgtggtgcagaaccacggcaaggaaaacgacaccctgcttggcgccgacacgccgcgcgcggcttccgccgaggtgcacgagcatgtgcacaagaacggcatgatgagcatgcagaaagtcgacagcgtcgacgtcgcaccaggcaaggacctgcgcttcgctcccggcggctaccacctgatgctgatgggcctgaagcagccactggtggccggcgagcgcttcccgctgacgctgcatttccgcaaggccggcgacgtgccggtggaaatcgtcgtcgagtccaaggcgccggccgaacagggcgggcacgagcagcacggccactga

SEQ ID NO: 49 - DNA, 312 bp, Pseudomonas aeruginosa
atgaacgacagcatctaccagcggattgataccaatccgcgcttcaaggagttggtggccaagcgagagcgtttcgcctggatactttcttcgatcatgcttggcctctacgtgatattcatcctgctgatcgctttccagccgcagctgctgggcgcgcgtatcagtcccgattcgtccgtgacctggggcatcccgatgggtgtcggactgatccttgccgcgttcatcctgaccggcctgtacgtgcgtcgcgccaacggcgagttcgacagcctgaaccaggaaatcctcaaggaggcgcagcaatga

SEQ ID NO: 50 - DNA, 198 bp, Pseudomonas aeruginosa
atgaattccgatgtgatcaaaggtaagtggaagcaactcaccggcaagatcaaggagcgttggggcgatctgaccgacgacgatctccaggctgccgacggacatgccgaatacctggtcggcaagctgcaggaacgctatggctggtccaaggaaagggccgaacaggaagttcgcgacttcagcgaccgcctgtga

SEQ ID NO: 51 - DNA, 237 bp, Pseudomonas aeruginosa
gtggtactgttcaacgacgattacacgccgatggactttgttgttgaagtgctggaagtgttcttcaacatggaccgggaaaaagccaccaagatcatgttgacggtacatactcagggtaaggcggtctgcggcttgtttacccgggacgtggccgaaaccaaggcgatgcaggtcaatcagtatgcacgggagagccagcatccattgctctgcgagatagagaaagacagttga

SEQ ID NO: 52 - DNA, 555 bp, Pseudomonas aeruginosa
atgagcgatgccaaggtcgatacccgccgcctggtcggccgcctgctgctggtgaccgtgctgatgttcgccttcggcttcgccctggtgccgctctatgacgtgatgtgccgcgccctggggatcaacggcaagaccgccggcagcgcctacagcggcgaacagcaggtggatgtcgggcgcgaggtgaaggtgcagttcatgacctcgaacaatatcgacatggtctgggagttccgctccgccggcgaccagttggtggtgcatcccggcgcggtgaaccagatggtgttctacgcgcgcaacccgagcgacaagccgatgaccgcgcaggccatcccgagcatcgccccggccgaggccgcagcctacttccacaagaccgaatgcttctgtttcacccagcaggtgctgcaacccggcgagagcatcgagatgccggtgcgcttcatcgtcgaccgtgatctgcccaaggatgtgcggcacgtgacgctggcctacaccctcttcgatatcactgcgcggaagccgcccgtaccggttgcgggacgataa

SEQ ID NO: 53 - DNA, 1923 bp, Pseudomonas aeruginosa
atgagcattttcagtcacttccaggaacgcttcgaagcgacccgccaagaggaatattccctccaggaatatctcgacctctgcaagcaagacaagacagcctatgcctctgccgccgagcgcctgctgatggcgatcggcgagccggaattgctggacacctcggtggactccaggctgtcgcggatcttctccaacaaggtgattcgccgctacccggcgttcgccgacttccacggcatggaggaatgcatcgaccagatcgtcgcgttcttccgccatgcggcccagggcctggaggagaagaagcagatcctttacctgctcggcccggtcggcggcggtaaatcctccctggcggaaaaactcaagcaactgatggagaaggtgcccttctacgcgatcaagggctcgccggtcttcgagtcgccgctgggcttgttcaaccccgacgaagacggcgccatcctcgaggaggactacggcatcccgaggcgctacctgcgctcgatcatgtcgccctgggcaaccaagcgcctcaacgagttcggcggcgacatcagccagttccgcgtggtcaagctgtacccctcgatcctcaaccagatcgccatcgccaagaccgagcccggcgacgagaacaaccaggacatatccgcgctggtcggcaaggtcgacatccgcaagctggaggaatacccgcagaacgacgcggacgcctacagctattcgggcgccctctgccgggccaaccagggcctgatggaattcgtcgagatgttcaaggccccgatcaaggtcctgcacccgctgctgaccgcgacccaggaaggcaactacaacagcaccgaaggcctcggtgccctgccctacagcggcatcattctggctcactccaacgagtcggaatggcacagcttccgcaacaacaagaacaacgaggccttcatcgaccgcatctacatcgtcaaggtgccgtactgcctgcgcgtggcggacgagatcaagatctacgacaagttgctggtcaacagctcgctggcgcatgcccactgcgcgccggacaccctgaagatgctctcgcagttctccgtgctgtcgaggctgaaagagccggaaaactcgaatatctactcgaagatgcgggtatatgacggcgaaaacctgaaagataccgatcccaaggccaagtcgatccaggaataccgcgactcggccggggtcgacgaaggcatggccgggctttccacccgcttcgccttcaagatcctctccaaggtattcaacttcgacccgcacgaggtagcggccaacccggtgcacctgctctacgtcctcgaacagcagatcgagcaggaacagttccagccggaaacccgcgagcgctacctgcgcttcatcaaggaatacctggcgccgcgctacgtcgagttcatcggcaaggaaatccagaccgcctacctggagtcctacagcgaatacggtcagaacatcttcgaccgctatgtgctgtacgccgacttctggatccaggaccaggaataccgcgacccggaaaccggcgagatcctcaaccgcgccgccctcaacgaggaactggagaagatcgagaaacccgcgggcatcagcaacccgaaggacttccgcaacgagatcgtcaacttcgtactgcgcgcccgcgccggcaacaacggcaagaacccgagttggctgtcctacgagaagctgcgcgtagtgatcgagaagaagatgttctccaacaccgaggacctgcttccggtcatcagcttcaacgccaaggccagcaaggaggaccagcagaagcacaacgacttcgtcaaacgcatggtcgagcgcggctacaccgagaagcaggtccgcctgctgtcggaatggtacctgcgggttcgcaagtcgcagtag

SEQ ID NO: 54 - DNA, 327 bp, Pseudomonas aeruginosa
atgatgaaaggtggcatggccggcctgatgaagcaggcgcagcagatgcaggaaaagatgcagaagatgcaggaagaactggccaacgccgaagtgaccgggcaatccggcgccggtctggtcagtgtggtgatgaccgggcgccacgacgtcaagcgcgtcagcctggacgacagcctgatgcaggaagacaaggaaatcctcgaagacctgatcgcggccgcggtcaacgatgcggtgcgcaagatcgagcagaacaaccaggaaaagatgtccggcttcacctcgggcatgcaactgccgcccggcttcaagatgcccttctga

SEQ ID NO: 55 - DNA, 1272 bp, Pseudomonas aeruginosa
atgagctacgtcatcgaccggcgtctgaacggcaagaacaagagcacggtgaaccgccagcgcttcctgaggcgctaccgtgaacacatcaagaaggccgtcgaggaggccgtcagccggcgttccattaccgacatggagcacggcgagcagatcagcattcccgggcgcgatatcgacgaaccggtgctgcaccatggccgtggcgggcggcagaccgtcgtccaccccggcaacaaggaattcaccgccggcgagcacatcgcccgtccatccggcggcgggggcggccgcggcggcggcaaggccagcaacagcggcgaaggcatggacgacttcgtcttccagatcacccaggaggaattcctcgacttcatgttcgaggacctggagctgcccaacctggtcaagcgccacatcaccggcaccgacaccttcaagaccgtccgcgccggcatcagcaacgacggcaacccgtcgcgcatcaacatcgtccgcaccctgcgctcggcccatgcgcggcgcatcgcgctgtccggcggcagccgggccaagctgcgcgcggcgctgaaggaactggagcggatcaagcgcgaagagccggacaacctcggcgatatccaggaactcgagctggaaatcgccaagctccgcgcgcgcatcgaccgggtgccgttcctcgataccttcgatctcaagtacaacctgctggtcaagcagcccaaccccacctccaaggcggtgatgttctgcctgatggacgtctccggctcgatgacccaggcgaccaaggacatcgccaagcgcttcttcatcctgctctacctgttcctcaagcggaactacgagaagatcgaagtggtgttcatccgccaccacaccagcgcccgcgaggtcgacgaggaggagttcttctactcccgcgagaccggcggcaccatcgtctccagcgcgctgaagatgatgcaggagatcatggccgagcgttatccgacccatgagtggaacatctacgccgcccaggcctcggacggcgacaactggaacgacgactcgccggtgtgccgggacatcctcctcaagcagatcatgccgttcgtccagtactacacctacgtcgagatcaccccgcgcgaacaccaggccctgtggttcgagtacgagcgcgtgcgcgaagccttcgaagacagcttcgcccagcagcagatcgtctcggcatcggacatctacccggtgttccgcgagctgttccagaggaggctcgtcgcatga

SEQ ID NO: 56 - DNA, 426 bp, Pseudomonas aeruginosa
atggctacacgacggaaaacaacaccccaggaaatcgatgatatccaggaccgcatgggttcgatgcgcgagctcgatttcgacgagcgccgccaggcgcgtaaggcgcggatcggcgacgagcggcccgaggccgaggtggaggccgaattttcctcgcggcgggtacgcgaggcgggccacgctggcgggcagccggacgaggacgatggttaccaggataacgtcggcatggacgatctggcgccggaaaccctgatcgacgaaagcggcgcccgctcgccggccgagcgcggcggcgaatcgcccgcggacaagcgcctgagggtcgtgcatggcaacgagatcggagccggccacggcctcgacgaggccgagctggcgcgtcgtgatccgctcgacggttcctccgacgaggaacgctga

SEQ ID NO: 57 - DNA, 387 bp, Pseudomonas aeruginosa
atgggcgaccgccttgccgtacccatccgctactacgccctggccgggatcgccgcggcgatcctgctcaacgtgctgctgcgcggggtggtccgcttcggcggcctgccggccagcctgctgatcgccgcgctggtcgccggcggcctggcctggtggttcgcccgcgcgcagcggcgctggccgacctggggcgagcgcttgcgcctggtcgccctgtatggcggagtgctcggggtcctttacctgctgctggtcggcctcgcctcgctgaaaggcgatcccagcccggcggcactgctcatcgtggtgctgcactacctgtgctatccggcgctgttgctggtgttcttctccggcagggtctatggattcttcctgcgctga

SEQ ID NO: 58 - DNA, 159 bp, Pseudomonas aeruginosa
atgagtctcggtacgattcttctgatcatcctgatcctgctgctgatcggcggcctgccggtctttcctcattcgcgcaactggggctacgggccctcggggatcatcggcgcattgctggtggtgctgttggtcctgttgctgctgggcatgatctag

SEQ ID NO: 59 - DNA, 477 bp, Pseudomonas aeruginosa
gtgttgaagttattggtcgtggttgcttccctgttcatcggcggaggcatggccatgggagagcctgggggggcggatgggttttccgctgtcgcagcggtcgatccgggagcgaaccccgaagcgcaactggacgaggagccccaggaggtcagcgaagcgccggcctatcgcgtggatgacctcaccttcctgtacctcacccatgaggtctatctggaaccctacgtttcctgccgtccgaaagtcctgggcgaacgttcgtacgtcgcctgctggaacgaaacctattccggccgctccccgctgaatttctgggaatacgacgggggcgacttcctcgccctcaacgacccggcacgtgtgttggccgagggaaagttcgccagcgagcagcgcatcggcgaagcgcccctgccgctgcccctggatatcgatctggaccagttggagcgggcctactcgctgatgatgtaa

SEQ ID NO: 60 - DNA, 2136 bp, Pseudomonas aeruginosa
atgtcaatagatattctggtaactaccgacagactaaacaaagatccgctaggtcgagaattcattagctatatagaaagtaatagtgaccgattaaacctggcgggaagtgctctttattacgattttccggcatactccgactacgacacggtaactcacaagccagatgtgcttatacttagtcccgcccacgggattttagcaatacgactaataaacctggcgagactaaatcaaggtgcactctcacaagccgatgagtcacttaatcaattctgcagcatactgattggccgcctgctaaaaagcaagccactaagagaagggagatcaaagctttcctttgaagtcacacccgttatatattgcgatcaaaaaacagatgtattacccgaagatcttgattgtgagctcgtgtcgtcgtatggcgcatttgaagccttaattgaaaaacttaaagacagagagtctaacgcagagactctttcagagattcgatctgttctggaaggtgcaaaagcgctaaccagagcctccaaaaggattgttgaagatccggaagttacaccagcagctgccgccctagccaaacttgagtcagaaattgccaactttgatcagaagcagagacgggcagcacttgtaacaatagatggccctcaacgaatacgaggcctggccgggtcgggaaaaacggttatcttggctatgaaggcagcacaccttcatatgacaaggcccaacgataaaatactggtaacttttttcacaaaaagccttagatcaccgattaaagacttagtaacaaagttctataggcattacaaagagattgatcccgactggaacaacatccacataaggcatggctggggaggtagcaatagcagcggaacctacgcagatgcttgcagaagatcaggtagaatgccccgctcatatagttcagcaagggatgctgcgcccaggggagtcgagccatttagcttcgcatgtgaggaattgatcaatagcgctcaaagccttgaatactacgaccatatactcatcgacgaagggcaggactttccagacgggttttataaactttgctttgaactggcaaagggcgatagagacaaaaaaaatatagtatgggcatatgatgagctgcaaaatattctaaatgtaaaaatgcgctctccagcagagctattcggacaggacgatgatggagagccccgtatctctctagctagggccgaaaagaatctaccgcctggagcaactaacgatacagtacttagtaaatgctatagaaaccaaagagaagttctcgtagcagcacatgccctagggtttgggatatattcaaatattgttcagctacttgaaagccctgatcattggcgtgacgttgggtatgatgtgctaacaccagattttcaagtcggaaatgacattgaaattcttagaccagtagagaatagccctgtagcccttgatacggatggcctcccccctttaataggtaacttcatagcaagcagttttgaaaaagaaataagctggataactaaagagataataaactttcttgatatgggacttcttccggaagatattattgtggtggcgttagatgaccgtcacatgagaaattatttccgttatttgtcagaaagtctcgcatcgcatgaaatatcagtgaataacatacatgcagacccctattcagaaccgccattcagtataagcgggaagatcacgctatctacggtgtatcgtgcaaaaggaaacgaggcggctgtggtttttgcgctaggtgttgatgctttgtctttacgccttcgcgctgatagaaacaagattttcgccgcttttacaagaacaaaggcatggctcagggtctcaggacttggtgattcagcaagaagagtagcagtcgaaatagacactgcccttaggaacttcccacaccttaaattcaaaatgccagacatttctgaaattgaccttatacaaagggatctaagcaagagaagtattaaggccaagaaaatcaggaatgaatatatacaaaaattgaaggacgaaggcttcaccgaggacgaaatcgcagacattctttctctggaggagaaagatgaatag

SEQ ID NO: 61 - DNA, 219 bp, Pseudomonas aeruginosa
atgcaagacctcggtttcgccctcctgctcatcggctacgtctggtccgtggccagcggcggccgccgttcgatcccctgcgcattgctctgcctgctgctgtttccgctggcgcaactggccttcgccatcaacgacgcgccgatgcgcccgccgctggcgctggcggcctttggcgcgggcctggcctacctcggcggcggctcggtgttcggctga

SEQ ID NO: 62 - DNA, 321 bp, Pseudomonas aeruginosa
atgaagttgtccgatagtttcgatgcgcgccggctgcgcccgcgccggccgagagtctggcgctggcgcctggcggcggcgttcgcggccctggtggcgagcctgggcgtgctgctgtcgctggccggcacctcggccttgatcggccgccagccggctgtcggcatgctgcatatcgaacccgccgctggcggcgtcttgctggcccttggcctgctgtcgctctgcctcggcgtgttcttctggcgtttcagccgccgccgcagccgccgcaacagcgacctgagcctgtcgccgaacctgatgaaaaagcgcgactga

SEQ ID NO: 63 - DNA, 315 bp, Pseudomonas aeruginosa
atgaccaccgttgctgacatcgtctccaccatgaaaagcaagttcaacgccagcgcggccgccggcctggatctggtgttccagttcaacatcgaagacggcgacaaccactacctggtcgtcaaggacggtacctgcgaagtggtacagggcgacgccgagaaccccaacgtcaccctgatcatggacagcgaaaccctgaagggcatcaccagcggcgagaccgacggcatgcaggccttcatggccggcaagctgcgcgccgaaggcgacatgatgctggcgatgaagctgggcgaactgttcccggtctga

SEQ ID NO: 64 - DNA, 288 bp, Pseudomonas aeruginosa
atgccgggctttttcatgcgctcgggaaactcaggcggcctggtggaacgggtccggctcgccgatgctgatgcgttcgtcggtctcaggcaggtcgaggtgttcatggccgcaggcggtgcagtacaccagccagcggtcgtcctggacattgaaggaaagatcgacgatcacggcctcgatctcgtccaggtggtcgatcagatagttatgttcgtggggtgcggtttccagcatgacgactctcctgtgttgtcgtttgcctgggtggggttctgtgaccggtaa

SEQ ID NO: 65 - DNA, 342 bp, Pseudomonas aeruginosa
gtgactctcagaaacggagtacccagcatgacgaaggatgaaaaggaaaagacccacgtcgacgcaatcatcgaacgctacaaggacctgatggtcgagattccccccgccgaccggcaacccggtctctccctgctctggccggttcccgcccagcccgccatcgacaagggcgtccgccaggccgagaactggcttgccgaccagatcgaaggccagctctggaccgccttcgccttcggccgcgacagcctgcccacaccgatgcagaagaccgccttcgaagtggccttcctgacgcgcctgcagcagcgcctggtagccgctcgacgttccggttga

SEQ ID NO: 66 - DNA, 222 bp, Pseudomonas aeruginosa
atgctgttgtccctgctgtgcctctccaccctggcgctcggcctcgccctcagcctcgccggcagtacccgcgaggaacgcgaacaggccgccctgctgccgttcgccgacgatcccgaggccgcccgccgggtcgcccgggataccggcaagatctgccggcaggtggtccgccccctcgaggaatcccgcgaggccgccggcccgccgttcctcgcctga

SEQ ID NO: 67 - DNA, 507 bp, Pseudomonas aeruginosa
atggacaagcgacttttgagcaaggggttggttctggggctgctgagcctgggcagcatgacggcacacgccgacgcggcaggcggtaatggctgtggttggggcaacatggtgttcgagggccagcgcggcctgttcccgcacctgctggccaccaccaccaacggtacctcgggcaacgccaccttcggcatgacctccgggaccaacggttgcaacaccaatgctcgcctgggctacggcggccgctcgatcttcgccatgaacgggatgctcgacaacatcgccgaggacatggccaagggccagggcgaagcgctcgacgcctatgccgtgctgctcggcgtggaagccaaggaccgggcgcacttcgcccaggtcacccagcagcatttcggcgagatattcgcgagcaaggatgcgacgggcgagcaggtcctgagcaacaccctggcggtgatgagtcgcgacggcaccctggcgcgctacgccaagcaaccggcctga

SEQ ID NO: 68 - DNA, 522 bp, Pseudomonas aeruginosa
atgaagaaactacttccgctggccgttctggccgcactttcctccgtccacgtggcgtccgcccaggcggcggacgtctcggccgccgtcggtgcgaccggccagtcgggcatgacctatcgcctgggcctgagctgggattgggacaagagctggtggcagacctccaccgggcgcctgaccggctactgggatgcgggctacacctattgggaaggtggcgatgaaggcgccggcaagcattcgctgtcgttcgctccggtattcgtctacgagttcgccggcgactcgatcaagccattcatcgaggccggtatcggcgtggcggcgttctccggcacccgtgtcggcgaccagaacctgggttcctccctgaacttcgaagaccgcatcggcgccggcctgaagttcgccaacggccagtcggtcggcgttcgggcgatccactattccaacgccggcctgaaacagccgaacgacggtatcgagtcctacagcctgttctacaagatcccgatctag

SEQ ID NO: 69 - DNA, 1368 bp, Pseudomonas aeruginosa
atgaaaacaacgaagattcttctgcataccggtgtactggccctgagcctgctggccacccaggtgatggcggcggtatccgccgacgaagcggccaagctcggcaccagcctgactccgttgggggcggagaaggccggcaacgccgacggcagcatccccgcctgggacggcggcctggcgaccaacgccggcagcgtcgacagccgtggcttcctcgccaacccctatgccagcgagcaaccgctgttcaccatcaccgcgcagaacgtcgaccagtacaaggacaagctgactcccggccaactggccatgttcaagcgctacccggatacctacaagatcccggtatacaagacccaccgcagcgccaccgtcccggcggcggtgcaggaggcggccaagcgcaacgccaccaccaccaagctggtggaaggcggcaacggcctggagaacttcgataccgccaacccgttcccgatcccgcagaacggcctggaggtgatctggaaccacatcacccgctatcgcggcggcagcgtccggcgcctggtgacccaggccactccgcaggtcaacggctcctaccagctggtctacttccaggatgccttcaccttccgtaccaacctgaaggactacaacccgaacaagccgagcaacgtgctgttctacttcaagcagcgggtcaccgcgccctcgcgcctggccggtaacgtgctgctggtccacgagaccctcaaccaggtcaaggagccgcgcctggcgtggctctacaacgccggccagcgccgcgtgcggcgggctccgcaggtgtcctacgacggtcccggcaccgccgccgacggcctgcgcacctcggacaacttcgacatgtacaacggtgcgccggaccgctacgactggaagctggaaggcaagaaggaaatctacattccctacaacagctacaagctcgacgatccgaagatcaagtacagcgaaatcgtcaaggccggccacatcaaccaggacctgacccgctacgagctgcatcgcgtctggcatgtggtcgcgaccctgaagccgggcgagcggcacatctacgccaagcgtgacttctacatcgacgaggacacctggcaggccgccgagatcgaccattacgacggtcgcggcaccctgtggcgcgtggccgaggcgcatgccgagcagtactacgacaagcaggtgccctggtacgcggtggaaaccctctacgacctgctctccggccgctacctggcgctgggcatgaagaacgaggagaaacaggcctacgacttcaactacagcgcttcggaaagcgactacaccccggcggcgctgcgccaggaaggggtacgctga

SEQ ID NO: 70 - DNA, 993 bp, Pseudomonas aeruginosa
gtgctggccgtgctgtcggtcagcctgatggtggtggacgcccggttcgactatctggagcccgtccgtagcaagctggggatggtgctcacgcctttctacggcctggcggaaatgccggtgcgcgcctgggaaggcgtgcgcgaccagttcagcagccgtagcgagctgatcgccgagaacgaacgcctcaaggccgagtccctgctgatgcagcgccgggtgcagaaactcgccgcgctcaccgagcagaacgtgcgcctgcgcgagctgctcaattccgccgcgctggtcgacgacaaggtactggtcagcgagctgatcggcgtcgatccgaaccccttcacccaacgcatcatgatcgacaagggcgagaacgacggcgtcttcgtcggccagccggtgctcgatgccagcggcctgatgggccaggtggtcgaggtgatgccctataccgcgcgggtcctgctgctcaccgacaccacccacagtatcccggtgcaggtcaatcgcaacggcctgcgcgccatcgccgtcggcaccggcaatcccgagcgcctggaattgcgctacgtcgccgacaccgccgacatcaaggaaggcgacctactggtcagctccggcctggggcaacgcttccccgccggctatccggtggccacggtcaaggaagtgatccacgattccggccagccgttcgccgtggtgcgcgcggtgccgaccgcgaagatgaaccgtagccgctacgtgctgctggtgttcagcgacagccgcaccccggaacagcgcgccaacgacgccgccgaggcccaggaagaggcggacaagaaagccgccgccggcgcccaggcgccccagcctgccgcgcaaccggccgccgcgccgtcgcccgccaccccggcagcccagggcgctgcgcagcagcccgccgccgcgcccgcgccagctcccacgcagcctgccgctccggcggcgaacggggggcgccgctga

SEQ ID NO: 71 - DNA, 273 bp, Pseudomonas aeruginosa
atgcgtaaaccagaactagccgccgctatcgccgaaaaggccgatctcaccaaggaacaggccaatcgcgttctcaacgccctgttggatgaaatcaccggcgcgctgaaccgcaaggacagcgtgaccctggtcggtttcggcaccttcctgcaacgccatcgcggagcccgcacggggaagaacccgcagaccggccagccggtgaagatcaaggccagcaacaccgtcgccttcaagccgggcaaggccctgcgcgacgcggtcaactga

SEQ ID NO: 72 - DNA, 2151 bp, Pseudomonas aeruginosa
atgagcagaaagcgtagccccgctccttcgcggatcagcgaagggcaccccttcccccttggcgccacctgggacggtctcggcgtcaatttcgcgctgttctcggcccacgcgaccaaggtcgagctgtgcctgttcgacgcgcgcggggaaaaggagatcgagcgcatcgaattgcccgagtacaccgacgagatctggcacggctacctgcccgacgcccacccggggcagatctacggctaccgggtgcacggtccctacgagccggatgccggccaccgcttcaatcccaacaaattgctcctcgatccctacgccaagcagttggtcggccgcctgcgctggtccgaggcgctgttcggctacaccatcggctcggccgacgccgacctcagcttcgacgagcgcgacagcgcgcccttcgtgcccaagtcgaaggtcatcgatccggccttcacctgggccgagcgtccgccggtacgcgtcccctgggaccgcacggtgatctacgaagcccacctgcgcggcctcagcatgcgccatccacaggtcccggaggcggttcgcgggaccttcgccgggctgatgaacgccgacctgctggcgcacatccgccggctcggggtgaccagcgtcgagctgctgccgatccacggtttcgtcgacgacaagcacctgctggaaaacggtatgagcaattactggggctacaacagcatcgccttcttcgccccgcacccggcctacctcgccagcggccaggtcaacgagttcaaggagatggtcgcgcacctccacgacgccggactggagctgatcctcgacgtggtctacaaccacaccgcggaaggcaacgagctgggtccgaccctgtgcatgcgcggcatcgacaacgcctcgtactaccgcctgatgcccgaccagcgccgctactacatcaacgattccggcaccggcaacaccctcgacctcagccacccctgcgtgttgcagatggtcaccgactcgctgcgctactgggccacggagatgcgcgtggacggcttccgcttcgacctggcgaccatcctcggccgccatccggacggtttcgacgagcgccacggcttcctcgtcgcctgccgccaggacccggtgctgagcaagtgcaagctgatcgccgaaccctgggattgcggccccggcggctaccaggtaggcggcttcccgcccggctgggcggaatggaacgaccgtttccgcgattgcgtgcgcgcctactggcgcggcgacgacggcatgctgccggaactggcgcggcgcctaaccgcctccggcgatctctacgaccagcgtggacggcggccctacgcgtcggtgaacttcgtcaccgcccacgacggcttcaccctgcgcgacgtggtttcctacgatcacaagcacaacgaggccaacggcgagaacaacgccgacggcagcgaccacaacctgtcctggaaccacggctgcgagggccccaccgacgacccggagatccgcgccctgcgcctgcgccagatgcgcaacctgctgtccaccctgctgctgtcccagggcaccccgatgctggtcgccggcgacgagttcagccgcacccagcagggcaacaacaatgtctattgccaggacaacgaactgggctggatcgactggcggctggacgacgagggccgttcgctgctggccttcacccagcgcctgctggccctgcgccaacgctatccgatcctgcgcagggggcgcttcctggtcggcgagtacaacgaggcgctgggtgtcaaggacgtcacctggctggcgcccggcggcgaggagatgaccgaggaacactggcacgacgagcatgcccgctgtctcggcgtgctcctcgatggccgcgcgcaacccaccgggatcctgcgcagcggtgaggatgccaccctgctgctgatcctcaatgcctaccacgacgcggtgtccttccgtcttcccgaggtggccgaaggcagcggctggacctgcctgctggatacccagcggccggaggacccgctcggcgaacgctacccgttcgccagcgagttcctggtcggcggccgcagtttcctgctgttcgagctacaaccgccgggcgccggagcggaaggatga

SEQ ID NO: 73 - DNA, 522 bp, Pseudomonas aeruginosa
atgccccatagctacaggaaaatggaatcgccggtcgggacactgaccctggtagccagggacgatgcctttctggtcgcgattctttggcagcatgagcgtcccaaccgagtaccgctggacgagatgcggctctccgaggacagctccctcctggcggaaaccgaacgccagttgcgggaatatttttccggcaaacgttcccggttcgaactcccgctcgacttccaaggtaccgaatttcagaaaaaggtctggtccgccctgctcaccattccattcggcgagacccgcagctatacggaaatagccgtgcagatcggcagccccaacgcagtacgggcggttggagccgccaacggacggaacccactgtccattgttgcaccttgccatagggtgatcggagcctctggcggcctcaccggtttcgccggtgggttggccgccaagcagtggttgctgcgtttggaaacccgaggcaggacaccagacctgttgagcatgatcgaggacgagtga

SEQ ID NO: 74 - DNA, 384 bp, Pseudomonas aeruginosa
atgcctatgcgaagcttgatcgttgcctgcctggcgctgagtgccaccggttgtaacagctggtcgctgaacagcgacctgaacggggcctaccgcgcctacgacaaaggcgattgcgcacaggtgatgctcgacctgtcgcgggccgaacggcggattcgcgcgcggccctacctgcagccggaaatctcgctgctgcgcggccagtgcctggaacgccagagcctgttcgtcgacgcggcgcagacctaccacttcatcatcgcccgttaccccaccagcgaatatgcctaccgtgccaaggcgcggctggagacgttgcgccagctgggccggctgagcgagacgccggcctcggccagcgccgtgccgacccgcctgtag

SEQ ID NO: 75 - DNA, 234 bp, Pseudomonas aeruginosa
atgaacctgaaaccccagaccctgatggtggcgatccagtgcgtcgccgcgcgcacccgcgaactcgacgcgcagttgcagaacgacgacccgcagaacgccgctgaactcgaacagttgctggtcggctacgacctcgccgccgacgacctgaagaacgcctacgagcaagccctgggccaatacagcggcctgccgccctatgaccggctgatcgaagagccggcatcctga

SEQ ID NO: 76 - DNA, 711 bp, Pseudomonas aeruginosa
atgaccgacccgatccgcctgtccaaacgcctcgccgaactcaccgcctgctcgcgccgcgaagccgagctgtacatcgagggcggctgggtcagcgtcgacggcgaagtgatcgaggagccacagttcaaggtgctcgaccagcgcgtggagctgctgccgggcgcccgcgcggaaacgatcgaaccggccaccctgctgttgcacaagccggccggctggcgccacgacgatttcgaaggcctgctcgctgcggggcggcgctggagcgatgaccccagtcccttgcgagcgctgaaaaaacacttcgcccgccagcgtcccacccttgcgctggacagcgaggcttcggggctggtggtgttcagccagcagcacggcgtactgcgcaagctggtggacgacggtgcacggctggagcaggaatacctggtcgaggtcgccggcgacctggccgccggcggcctcgagcgcctgcgccacggcctcgcctaccagggccgcaggctatcgccgtgcaaggtcagttggcagaacgaaagccacctgcgcttcgccctcaaagacgtgctgcccggccagttgcgcttcatgtgcgaaagggaggggctggaagtgcggagcatccgccgcctgcgcatcggcgcgctgtcgctggccaggctaccgctcggcgaatggcgctacctcggcctccacgaacgcttctga

SEQ ID NO: 77 - DNA, 231 bp, Pseudomonas aeruginosa
atgctgatcccccacgacctgctcgaagccgacaccctgaacaacctgctggaagacttcgtcacccgcgaaggcaccgacaatggcgacgagaccccgctggacgtgcgtgtagaacgcgcccggcatgccttgcggcggggcgaggcggtgatcctgttcgacccggagagccagcagtgccagttgatgttgcgcagcgaggtgcctgcggagctgttgcgcgactga

SEQ ID NO: 78 - DNA, 294 bp, Pseudomonas aeruginosa
atgaagcttcgtcctctgcatgatcgcgtcgttatccgtcgcagcgaggaagagaccaagaccgcaggcggcatcgtgctgccgggttccgccgccgagaagccgaaccgcggtgaagtggtagccgtaggtaccggtcgtgtactggacaacggcgaagtgcgcgctctggcagtgaaggtgggcgacaaggtggtcttcgggccttactccggcagcaacgccatcaaggtcgatggcgaggaactgctggtgatgggcgagtccgaaatcctcgccgtcctggaagactga

SEQ ID NO: 79 - DNA, 369 bp, Pseudomonas aeruginosa
atgcgttcctggatctatctgttactggctatcggcgccgaagtgatcggcaccacctcgatgaaactggccgccacccacgcccctgtcgcaggcatgctgctgatgtacgggatgatcggcctgtcctatttcttcctcgccctggcggtcaagcgtgtccccgtcggagtcgcctacgccctttgggaaggcattggcatcgtcctgatcacggcggtcagcgtcgcctggctgggcgaaagcatcggcctgtacaaggccgtcggcctcggcgtgatgatcgccggcatcctgctgatcaagtccggcacccgcaacgccagcggcacgccggcgcagtcccgtggggaggccgtcacatgctga

SEQ ID NO: 80 - DNA, 2379 bp, Pseudomonas aeruginosa
atgaacagcgccacgctaaccgagctcgatctgccggtcagcggcatgacttgcgcgtcctgcgccggccgtgtcgaacgagccctgaagaaagtgcccggggtcgcggccgcctcggtcaacctggccagcgagcaggcccgcgtacaggccccggcggacagcctgccggccctggtggcggcggtcgagcaggccggctaccaggttccggcgcgcagcctggaactgtccatcgagggcatgacctgcgccagttgcgtcggccgggtcgagcgggcgctgaagaaggtgcccggcgtacgcgaggtcagcgtcaacctggccagcgagcgcgcccacctcgagctgctcggcgccgtggacagccaggccttgctacaggcggtggaacaggccggctacaaggcccgcctgctcgacgcggggcaaccacgccaggacgatgccgagcgccgcctgcgccgcgaacgctggtgggtgatcgccgcgctgttgctggcgttgccgctggtgctgccgatgctggtggaatgggccggcctgcactggatgctgccgccgtgggcgcagttcctcctggcgaccccggtgcagttcgtcatcggcgcgcgcttctatgtctccgcctggcgcgcggtgaaggccggtgccggcaatatggacctgctggtcgccctcggcaccagcgccggctacggcctcagcgtctacctctggctgaccgccccgcccggccacatgccgcacctctacttcgaagcctccaccgtggtgatcgccctgatcctcctcggcaagtacctggaaagccgcgccaagcgccagaccgccagcgccatccgcgccctcgaggcgttgcgcccggagcgcgcggtgcgcctgcgcgacggtcgcgaggaagaggtggcgatcgccgagttgcgcctcggcgacgaggtggtggtgcgtcccggcgaacgcttcccggtggacggcgaggtgctcgacggcagcagccacgccgacgaggcgctgatcaccggcgagagcctgccggtgcccaaggctcccggcgacaaggtcaccggcggcgcgatcaacggcgaaggccgcctgctcctgcgcaccactgcgctcggcggggaaacggtgctggcgaagatcatccgcctggtggaagacgcccaggccgccaaggcgccgatccagaagctggtggacaaggtcagccaggtcttcgtcccggtggtgatcctcatcgcgctggtcaccctcggcgcctggctggtcgccggggtcggcctggagcaggccctggttaacgccgtggcggtgctggtgatcgcctgcccctgcgccctcggcctggccaccccgaccgcgatcatggccggcaccggcgtggccgcgcgccacggcatcctgatcaaggacgccgagtccctggaagtcgcccatgcggtcaccagcgtggccttcgacaagaccggcaccctgacctccgggcgaccgcagatcatccacctcggcggcgacgaccaggagcaactgctcaggctcgccggcgccctgcaacgcggcagcgagcacccgctggccaaggccgtgctcgagcgctgcgccgagcgcgacctggaggttccgccggtgaacgccagccaggccctcagcggtcgcggcatccagggcgaggtcgagggtcgccggctggccctgggcaaccgccgcctgctcgacgagcaggaactcaagcccggcgcgctggcgagtgccgccgcggactgggaggccgagggccgcaccctgtcctggctgctggaattggctccggagaaacgcgtgctcggcctgttcgccttcggcgacagcctcaaggacggcgccgccgaagcggtggaggccctgcgcggacgggatatccacagccacctgatcaccggcgacaaccgcggcagcgcggcggtggtggccaaggccctgggcatcgacgacgtacacgcggaagtgctgccggcggacaaggccgccacggtcgccgagctgaaaggccggggccgggtggtggcgatggtcggcgacggcatcaacgacgccccggcgctggccgctgccgacgtaggcatcgccatgggtggcggcaccgacgtggccatgcacgccgccggcatcaccctgatgcgcggcgacccgcgcctggtgccggcggcgctggacatctcgcggcggacctacgcgaagatccgccagaacctgttctgggccttcatctacaacgtgatcggcatcccgctggcggccttcggcctgctcaacccgatggtggcgggcgcggcgatggccttctccagcgtcagcgtggtcggcaacgccctgctcctgcgacgctggaagccctga

SEQ ID NO: 81 - DNA, 639 bp, Pseudomonas aeruginosa
atgcgacgcacaaaggaagattctgaaaaaacccgtacggccatcctcctggccgccgaggaactgttcctggaaaagggcgtgtcccataccagcctggaacagatcgccagggccgccggggtgacccgtggcgccgtctactggcacttccagaacaaggcccacctgttcaacgagatgctcaaccaggtacgcctgccgccggagcaactcaccgagcgcctgtccggctgcgatggcagcgacccgctgcgctcgctctacgacctctgcctggaggccgtgcaatcgttgctgacgcaggagaagaagcgccgcatcctgaccatcctgatgcaacgttgcgaattcaccgaggaactgcgcgaggcgcaggaacgcaacaacgccttcgtgcagatgttcatcgaactctgcgagcagttgttcgcccgcgacgaatgccgtgtgcggctgcatccgggcatgaccccgaggatcgcctcgcgcgccttgcacgcgctgatcctgggcctgttcaacgactggttgcgcgacccgcgcctgttcgatccggatacggacgcggaacacctgctggagccgatgttccgtggcctggtgcgcgactggggtcaggccagctcggcgccgtag

SEQ ID NO: 82 - DNA, 633 bp, Pseudomonas aeruginosa
atgctggtctgcgcagcattcagcggaggcgccggcgcgagcgacgccggagcattgctcgaagccgcgcgtcgcggcgataccgcgcaggtcgccagcctgctcgaacgcaaggtcgaggtcgacgcgcccagcgccgacggtagcaccccgctgctgctggccaccgccaacgaccacctggcggtggcgcgccagttgatcgaggccggcgccgacgtcaaccggcagaacagccggctcgacagcccgtatctgctggccggcgccgagggccgcctggaaatcctgcgcctgaccctgctccacggcgccgacctgaagagtaccaaccgctatggcggcaccgcgctgatcccggcctgcgaacgcggccatgtggaagtggtgaagacgctgctgcaagccggggtcgatcccaaccatgtcaacaagctcggctggaccgggctgctcgaggcgatcctgctgagtgacggcggtccgcgccaccaggagatcgtgcgcctgctgatcgacgccggcgccgacgtcaacctggccgatgccgacggcgtcagcccgctggcccacgcccgccagcgcggccagggcgggatcgagcggctgctgctggccgccggcgcccaatga

SEQ ID NO: 83 - DNA, 1914 bp, Pseudomonas aeruginosa
atgggcaaaatcattggcatcgacctgggtaccaccaactcctgcgtggctatcctggagaacggtaacgtcaaggtcatcgagaacgccgagggcgcgcgtaccaccccctcgatcatcgcctacaccaacgatggcgaaaccctggtgggccagccggccaagcgccaggcggtcaccaacccgcagaacaccctgtatgcggtgaagcgcctgatcggccgtcgcttcgaagagaacgtggttcagaaagacatccagatggtgccgtacagcatcgtcaaggccgacaacggcgacgcctgggtggaagtgaagggccagaagatggcgcctccgcagatttccgccgaagtgctgaagaaaatgaagaagaccgccgaagactacctcggcgagccggtcaccgaagcggtgatcaccgttccggcctacttcaacgacagccagcgccaggccaccaaggacgccggccgcatcgccggcctcgacgtcaagcggatcatcaacgagccgaccgcggcggcgctggcctacggcctggacaaggccaagggcgaccacaccgtgatcgtctatgacctgggcggcggtaccttcgacgtgtcggtgatcgagatcgccgaagtcgacggcgagcaccagttcgaagtgctggccaccaacggcgacaccttcctcggcggcgaagacttcgacatccgcctgatcgactacctcgtcgacgagttcaagaaggaaagcggcatcaacctgaagggcgacccgctggccatgcagcgtctgaaggaagcggccgagaaagccaagatcgagctgtcctcgacccagcagaccgacgtcaacctgccgtacgtgactgccgacgcgagcggtccgaagcacctgaacgtcaaggtttcccgcgccaagctggagtccctggtcgaagacctggtgcagcgcaccatcgagccgtgccgcaccgcgctgaaggatgccggcctggatgtttccgatatccatgaagtgatcctggtcggcggccagacccgcatgccgctggtacagaagaccgttgccgagttcttcggcaaggaagcgcgcaaggacgtcaacccggacgaagccgtggctgtcggtgccgccatccagggcgcggtcctggccggcgacgtgaaggacgtgctgctgctcgacgtgaccccgctgaccctcggcatcgagaccctgggcggcgtgatgaccggtctgatcgagaagaacaccaccatcccgaccaagaagtcgcaggtgttctccaccgccgacgacaaccagggcgcggtgaccatccacgtgctgcagggcgagcgcaagcaggccgcgcagaacaagtcgctgggcaagttcgacctggccgacattccgccggctccgcgcggcgtgccgcagatcgaggtgaccttcgatatcgatgccaacggcatcctgcacgtctcggcgaaggacaaggccaccggcaagcagcagtccatcgtgatcaaggcatcctccggcctgtccgaggatgagatccagcagatggtccgcgatgccgaggcgaacgccgaggaggaccgcaagttcgaggaactggctgccgctcgcaaccagggcgacgcgctggtccacgcgacccgcaagatgatcaccgaggcgggcgacaaggccaccgccgaggacaaggcgaccatcgagaaggcgctgggcgagctggaagcggcggtgaagggcgacgacaaggccgagatcgaggccaagatgaacgctctgtcccaggcttccaccccgctggcgcagaagatgtacgccgaacaggcccagcagggcgaagacgctccccagggcgagcaggcgaaagccgctgacgacgtggtggacgccgagttcgaagaggtcaaggacaacaagtaa

SEQ ID NO: 84 - DNA, 549 bp, Pseudomonas aeruginosa
gtgaacaagtcgatgctcgtcggggccgttctgggtgccgtaggcgtgaccgcaggtggcgcggtagccacctatagcctggtggatcgcggtcccgactacgccgaggtcgtcgctgtccagccggtcaaggaaaccatcaagacgccgcgccaggtctgcaaggacgtggccgtgacacgccagcgtccggtcaaggaccagcaccagatcgccggtaccgccatcggtgcggtggtcggcggcttgctgggcaaccagatcggcggcggtaccggcaagaagatcgccaccgtggccggcgccgtcggcggcggctacgccggcaacaaggtgcaggaaggcatgcaggagcgtgacacctacaccaccaccgaaacccgctgcagcaccgtgcacgacagcagcgagaaggtcgtcggctatgacgtgaagtacatgctcgacggcaaggccgggcagatccgcatggagcgcgatccgggcagccagatcccggtcgacaagaatggccggctgatcctcagccagggcgaaacgctgcgctga

SEQ ID NO: 85 - DNA, 2430 bp, Pseudomonas aeruginosa
atgtcgaagaacgcacgttacgcctggcgcctgtccctcggcggcctgctgctggggctactggcctgcgcggtctacctgctggccgtcccgctgggcttcaagtacggcgagatccaggtcggccatggcctggaacacgaggggcggatcgcctgggacgccgccggcgtcccgcatatccgcgcgcagagcctggaggacggctatttcctgctcggctacagccacgccagggatcgcctgtggcagatggagttcgcccgccgctatgccggcgggacgctctccgaagtgttcggcgccaagaccctgccgatggacaggttcgcccgcaccctgggcttccgacgcaccgccgaaggcatctacgccaacctggacgcgcccacgcgcgtcctgctgcaacgctacagcgacgggatcaacgcctatctggagctggccccggcggcgctgccgctggaattcagcctggtgcgccacgaacggccggggccctgggggccggtggacagcctgtccctgcacctgctctattcctggaccctgagcgccaacctgggcatgcagttgcagcgcctggcgctggccgagcacctggacctggcgcggatcaacgaggtgttcgccccctatccgggcgagcggccgccggccacccgcgactacgccagcctgtaccgctccctgcacggcacgccggacgccggcaagctgctggggcaactgcccggttccaacgtcgaaggcatcggttcgaacaactgggtggtctccgccagccgcagcgccaccggcaagccgctgctggccaacgatccgcacctgcgcctgaccaacccggcggccttctacctggccagcctgaagatccccggcctaagcctcaccggcgccaacttcgccggcgccccgctgttcgtcatcggccataaccagcgcatcgcctggggctacaccaataccggctcgcacatccaggacgcctacctggagcgcgtcgatccccaggacccgcggcgctacctgaccccggacggctaccggcccttcgagacgcgcctggaacgcatcgccgtgcgcgacggcgagacggtctcgctggaggtccgcagcacccgccacggcccggtgatcagcgacatctacgaaccggcgcggctgccgcaggcgcagcgcgaccggctggtgatcgccctcgcctggaccggcctcgaccgtcacgacaagaccttcccgagcctgctggcgatcaaccgggccgaaggctgggaacagttcctcgacgccgccgccaacttcggcgtaccgccgcagaacatggtctatgccgacgtcgagggcaacatcggctacgtctccgccggtcgcgtaccgctccgcggcgccgacgacgacctccatggcctggccccctcgcccggctgggagagccgctacgactgggtcggctacgttccggaaagcgccaagccacgcagcctcaacccccgcgaagggttcatcgccacggcgaaccagcgcatcgtgccgcccgacaatgccttcgacttcggccacgactgggtcctgccctaccgctatgaccgcatccgcgagtggctcggcggccccggccagcgcaccctggaagacagcctggagttgcagaacgacgagttctccagcgtgatggcgagcctgttgccgaagatgctggagcaggtcagcgaccccgaactgcgcgccagcgaggccttcgccctgctccagggctggaaccaccaggccgccgccgacctggccgcgccactgatcgccggctactgggtacgcgccttcacccgcgagctgctgcaaccgaggatcggcacgcagttgctggccagcggctggaaccagcgcaactacgacggtttcctccggctgatcctcgacggccaggccgacctgcgcttctggtgcggccaggaacaaggctgcgacctcaagctgaaccagtcgttgcgccgcgccctcgacgaactgcgtgccgcccacggaagcgcgccgagcggctggaagtggggcgaggcgcacgccgccctcgccgagcacgtgcccttccacaagaccccgctgcgcgcgctgttcgacctgaagaacaacaagggcggcgacaacttcagcgtcaacgtcgggcgcttcgactacagcgacccggccaacccgttcaacacccggatcgccgcgaccctgcggatggtcatcgacctggcggacttcgacaactcgcgctacgcgctgtccacccgcaactccggcctgccgttcgacggcgccaccgacctcaacgaactctgggcccgcggcgcgtacatccgtatcgccgacgacgcccccgacgcgacggaccgccagttggtcctgcgcccttcagcctcttcctccggcgagccacgaccatga

SEQ ID NO: 86 - protein, 82 aa, Pseudomonas aeruginosa
MKAMKQRIAKFSPVASFRNLCIAGSVTAATSLPAFAGVIDTSAVESAITDGQGDMKAIGGYIVGALVILAVAGLIYSMLRKA

SEQ ID NO: 87 - protein, 83 aa, Pseudomonas aeruginosa
MSGVVAVQVCTAWTSTPEGFMACRELAWQQAYLIPPEAAGYVDILVNGGFSPEAFGIGAAGVLGSFVTGLLIGWVASLLRKAK

SEQ ID NO: 88 - protein, 144 aa, Pseudomonas aeruginosa
MNMFATQGGVVELWVTKTDTYTSTKTGEIYASVQSIAPIPEGARGNAKGFEISEYNIEPTLLDAIVFEGQPVLCKFASVVRPTQDRFGRITNTQVLVDLLAVGGKPMAPTAQAPARPQVQAQAPRPAQQPQGQDKQDKTPDAKA

SEQ ID NO: 89 - protein, 30 aa, Pseudomonas aeruginosa
MLRYLSLFAVGLATGYAWGWIDGLAASLAV

SEQ ID NO: 90 - protein, 96 aa, Pseudomonas aeruginosa
MAASPYYLRQTHAPDCACSVCWSARQVIPLHSPSPCPDCRPPGLPYLEGGRWLCRPRSFCAKHDPSRRPPKYWHVVYDSGKPTPFVPVREAFQLEG

SEQ ID NO: 91 - protein, 430 aa, Pseudomonas aeruginosa
MKKISHQIRVSIESDGQVLESPKGRLFFDDTTAQFTDLSGVRILRCGVDTVRQLYNGKLRPEVMALFDLSVDVVEFAGYEWSKGRIGRDSGYQYRLQNAEMGLILLIKNHNIKVDTIGSHLKIEVSPHALDGADPRILQGVLDDLAAAVLSHCETNQAAVHIALDVQGWKPPRDLVDRMHCRSRRVRQISGIERIEFDGNASVYGRGETYMFGSANGLQLSIYNKTLQARATDKLDYWESVWATLNGDPFGDGDPAYNPLETVWRLEFRFHHSIVQQFSEGSRMASGEVIGCRTYEGLCPHLQGLWNYACESFKLLSRTAVYDPFWSLISQDARVQVECDPLIERTEYRRYYKTAKGFSGRNCEMFLGQFVSLIARERVPAKKAIESARKLEFWHVIEDHYLAKGWTRRDLERHIHKLMCDRYLRRGYAV

SEQ ID NO: 92 - protein, 420 aa, Pseudomonas aeruginosa
MWGLTMKFASLILMLLFATVARAEDYYWKIQSLPERFSSPSAACAAWAKATGRPGEFTFTGSMKARDQTSFWCEFTNNETGKTAAGYGPAGRYGDSCPEGTEYDKATGVCKSPPQECKEGELFPAKGPDSPVVTSGGRNYVGDGGAPTACYQSCEYGGNPSPASCYLVKGSTTTGFCNYILKGTGQNCGADSYTFSQTGDSLNPPDTPNTDPSDPNDPGCPPGWSWSGTTCVKAPTDPTDPTDPTTPGSDGGGDGNGGGNNNGGGNDGGTGNGGDGSGGGDGNGGGDGSGDGDGSGTGGDGNGTCDPAKENCSTGPEGPGGELKEPTPGTWDDAIATWEKKVEDAKQELKTKVKANVDQMKGAFDLNLAEGGGQLPCESMTIWGKSYSLCISDYAGQLSSLRVALLLMAALIAALILLKD

SEQ ID NO: 93 - protein, 118 aa, Pseudomonas aeruginosa
MEWLSGFLDQIIAFFQWIWDFFAQGIYDFVRDGLVVATKASMYAALQTLILLIDVSYTAARELIDSLGVPQMIRSMYAALPGPIAAGLAFFGVPQALNIIMVAAATRFCMRFVPFIGR

SEQ ID NO: 94 - protein, 424 aa, Pseudomonas aeruginosa
MSIKIHHGPNGSYKTSGAIQDDAVPALKDGRVIITNVRGFTLERAYQVFPDLPNTAEIINLDLESLEDLEKMRTWFQWAPRGAFLIFDETQLLFPKSWREKDLERFDYPGGPEAAHAADRPMGWLDAWTRHRHFNWDIVLTTPNISYIRDDIRMTCEMAYKHSNLAVIGIPGRYKEAQHDAQLNRPPADGTIIEYKRIRKQTFALYQSTATGKTQDTKAGKSLFRSPKLVLLLALLAGTIGFVWYMGPLRTIGAPAAATPADAPGDPAQAPAAPAAVAAPTRPAANSFLPPGLVPDGPAAAPVDLNAHPFADRRISILAHAYRKSRGDIYMFALDDPTGRRLELTSWQLIGSGYRVTPKGECVVELRYEEWKQTVTCTGRQPGAVASIVPAAPVAASADAPARGQSPLTIVPDSEYASRPWRQK

SEQ ID NO: 95 - protein, 183 aa, Pseudomonas aeruginosa
MTRTSNPCAVVLAFAAIAASGTAMAANTITFSGEVTDQTCQVAVNGFTDPTVILDSVPVSALDGAVGRSAGETAFTLQLTDCVAPTADEHFTTLFQATNPSAAGNLVNTAASGATGVALQLLDSVGGNPVDLAGGAAVPAGDIVLANGATSTSYDYAVQYVSEAATVTPGPVLGVVTYTLRYE

SEQ ID NO: 96 - protein, 149 aa, Pseudomonas aeruginosa
MKAQKGFTLIELMIVVAIIGILAAIAIPQYQNYVARSEGASALATINPLKTTVEESLSRGIAGSKIKIGTTASTATETYVGVEPDANKLGVIAVAIEDSGAGDITFTFQTGTSSPKNATKVITLNRTADGVWACKSTQDPMFTPKGCDN

SEQ ID NO: 97 - protein, 237 aa, Pseudomonas aeruginosa
MSIDNVSGTSSNTGNVNGSKRAAGSGATETGQSVKGSSNLGKDEFLKLLVAQLKNQDPMSPQQNGEFIAQLAQFSTVEGVQSLNKSMESILSNYQSSQALQASSLVGRKVIVATDKSVVDTKDTFKASLNLPVSSSNVWVNVYDDKGTVVNRINLGQQAAGSVSFMWDGKDSSGNIMPPGTYKFEAQTSIDGKTYGLQTYLPANVDSVTLGQNGGELMLNLAGLGSIALSKVQIIGQ

SEQ ID NO: 98 - protein, 248 aa, Pseudomonas aeruginosa
MPRHSYRSGRTSALLLVLLASIAQARASVVVTGTRVIYPGEAREKTVQLSNRDAFPNVVQAWVDIDAPDAPPDQADAPFLVNPAVFRMAPDSGQTLRIVYTGQGLPGDRESLFHLNVLQIPPRNSSHADRNQMLLMLRNRLKLFYRPAGIQGRPEDLPGQLRFALVRRSAGWAVRVDNPSGYYASFASATLSVGQRRWRLRSGMLEPRSHAEWQAETREALPPGRVRLHALLINDYGAHMDIRHDLSP

SEQ ID NO: 99 - protein, 488 aa, Pseudomonas aeruginosa
MALTVNTNIASLNTQRNLNASSNDLNTSLQRLTTGYRINSAKDDAAGLQISNRLSNQISGLNVATRNANDGISLAQTAEGALQQSTNILQRIRDLALQSANGSNSDADRAALQKEVAAQQAELTRISDTTTFGGRKLLDGSFGTTSFQVGSNAYETIDISLQNASASAIGSYQVGSNGAGTVASVAGTATASGIASGTVNLVGGGQVKNIAIAAGDSAKAIAEKMDGAIPNLSARARTVFTADVSGVTGGSLNFDVTVGSNTVSLAGVTSTQDLADQLNSNSSKLGITASINDKGVLTITSATGENVKFGAQTGTATAGQVAVKVQGSDGKFEAAAKNVVAAGTAATTTIVTGYVQLNSPTAYSVSGTGTQASQVFGNASAAQKSSVASVDISTADGAQNAIAVVDNALAAIDAQRADLGAVQNRFKNTIDNLTNISENATNARSRIKDTDFAAETAALSKNQVLQQAGTAILAQANQLPQAVLSLLR

SEQ ID NO: 100 - protein, 474 aa, Pseudomonas aeruginosa
MAGISIGVGSTDYTDLVNKMVNLEGAAKTNQLATLEKTTTTRLTALGQFKSAISAFQTALTALNSNAVFMARTAKSSNEDILKASATQSAVAGTYQIQVNSLATSSKIALQAIADPANAKFNSGTLNISVGDTKLPAITVDSSNNTLAGMRDAINQAGKEAGVSATIITDNSGSRLVLSSTKTGDGKDIKVEVSDDGSGGNTSLSQLAFDPATAPKLSDGAAAGYVTKAANGEITVDGLKRSIASNSVSDVIDGVSFDVKAVTEAGKPITLTVSRDDAGVKDNVKKFVEAYNTLTKFINEQTVVTKVGEDKNPVTGALLGDASVRALVNTMRSELIASNENGSVRNLAALGITTTKDGTLEIDEKKLDKAISADFEGVASYFTGDTGLAKRLGDKMKPYTDAQGILDQRTTTLQKTLSNVDTQKADLAKRLAALQEKLTTQFNLLSAMQDEMTKRQKSITDNLASLPYGSGKKT

SEQ ID NO: 101 - protein, 462 aa, Pseudomonas aeruginosa
MSFNIGLSGIQAASSGLNVTGNNIANAGTVGFKQSRAEFADVYAASVLGSGSNPQGSGVLLSDVSQMFKQGNIDSTNSVLDLAINGNGFFVTSNNGAISYTRAGYFNTDKQDFIVDNNGYRLQGYAVGPNGQLQNGVVTDLKVERANQAPQATSSIQQSYNLNSTLKPPTVTPFDPSDAATYNSSSSLGIYDSQGNSHTMSQFFIKNEPDPNATPPIPENSWTMKVLIDGVNPLDPSNKTPMSFNVTFDASGQMTSVRAPDGSTSGPGFSIDATTNVIQFSPATGNPPTPGTGWIPAASDGKTPPTYAWNGATGAASGISFDMRKTTQYSTAFAQSNPIQDGYTTGQLAGLEIDDTGVIFARYTNGQSKVQGQVVLANFANIQGLTPIGKTSWVQSSESGEPAVGAPRSGTLGALQSGALEASNVDISNELVNLIVHQRNYQANAKTIQTEDAVTQTIINLR

SEQ ID NO: 102 - protein, 78 aa, Pseudomonas aeruginosa
MSRVCQVTGKGPVTGNNISHAHNKTRRRFLPNLQHHRFWVESEKRFVRLRVSAKGMRIIDKRGIEAVLADLRARGEKF

SEQ ID NO: 103 - protein, 116 aa, Pseudomonas aeruginosa
MTNKIIQQIEAEQMNKEIPAFAPGDTVIVQVKVKEGDRQRLQAFEGVVIAKRNRGLNSAFTVRKISNGVGVERTFQTYSPIVDSLSVKRRGDVRKAKLYYLRALSGKAARIKEKLV

SEQ ID NO: 104 - protein, 200 aa, Pseudomonas aeruginosa
MQLNVNGAQAIEVSERTFGGEFNETLVHQAVVAYMAGGRQGSKAQKTRSEVSGGGKKPWRQKGTGRARAGTIRSPIWRGGGTTFAAKPRSHEQKLNKKMYRAALRSILAELVRLDRLVVVADFAVDAPKTKGLVAKLDTLGLKDVLIVTDGVDENLYLAARNLAHVDVRDVQGSDPVSLIAYDKVLVTVSAVKKFEELLG

SEQ ID NO: 105 - protein, 116 aa, Pseudomonas aeruginosa
MSVKKETRLRRARKARLKMRELETVRLCVYRSSQHIYAQVIAADGGKVLASASTLDKDLREGATGNIDAAKKVGQLVAERAKAAGVTQVAFDRSGFKYHGRVKALADAAREGGLEF

SEQ ID NO: 106 - protein, 99 aa, Pseudomonas aeruginosa
MNQERVFKVLLGPHISEKATGLADGKSQFVFKVATDATKLEIKKAVESLFSVKVQRVTTLNVKGKTKRTARGLGKRNDWKKAYIALQPGQDLDFATSAE

SEQ ID NO: 107 - protein, 156 aa, Pseudomonas aeruginosa
MPRRRVAAKREVLADPKYGSQILAKFMNHVMESGKKAVAERIVYGALDKVKERGKADPLETFEKALDAIAPLVEVKSRRVGGATYQVPVEVRPSRRNALAMRWLVDFARKRGEKSMALRLAGELLDAAEGKGAAVKKREDVHRMAEANKAFSHYRF

SEQ ID NO: 108 - protein, 840 aa, Pseudomonas aeruginosa
MTQVTVKELAQVVDTPVERLLLQMRDAGLPHTSAEQVVTDSEKQALLTHLKGSHGDRASEPRKITLQRKTTTTLKVGGSKTVSVEVRKKKTYVKRSPDEIEAERQRELEEQRAAEEAERLKAEEAAARQRAEEEARKAEEAARAKAAQEAAATAGAEPAVVADVAVAEPVAKPAAVEERKKEEPRRVPKRDEDDDRRDRKHTQHRPSVKEKEKVPAPRVAPRSTDEESDGYRRGGRGGKSKLKKRNQHGFQNPTGPIVREVNIGETITVAELAAQMSVKGAEVVKFMFKMGSPVTINQVLDQETAQLVAEELGHKVKLVSENALEEQLAESLKFEGEAVTRAPVVTVMGHVDHGKTSLLDYIRRAKVAAGEAGGITQHIGAYHVETERGMVTFLDTPGHAAFTAMRARGAQATDIVILVVAADDGVMPQTQEAVQHAKAAGVPIVVAVNKIDKPEANPDNIKNGLAALDVIPEEWGGDAPFVPVSAKLGTGVDELLEAVLLQAEVLELKATPSAPGRGVVVESRLDKGRGPVATVLVQDGTLRQGDMVLVGINYGRVRAMLDENGKPIKEAGPSIPVEILGLDGTPDAGDEMTVVADEKKAREVALFRQGKFREVKLARAHAGKLENIFENMGQEEKKTLNIVLKADVRGSLEALQGSLSGLGNDEVQVRVVGGGVGGITESDANLALASNAVLFGFNVRADAGARKIVEAEGLDMRYYNVIYDIIEDVKKALTGMLGSDLRENILGIAEVRDVFRSPKFGAIAGCMVTEGMVHRNRPIRVLRDDVVIFEGELESLRRFKDDVAEVRAGMECGIGVKSYNDVKVGDKIEVFEKVEVARSL

SEQ ID NO: 109 - protein, 70 aa, Pseudomonas aeruginosa
MRRLKRDPLERAFLRGYQNGITGKSRDLCPFTHPTTRQSWLNGWREGRGDNWDGLTGTAGLQRLNQLQHV

SEQ ID NO: 110 - protein, 758 aa, Pseudomonas aeruginosa
MLNRELEVTLNLAFKEARAKRHEFMTVEHLLLALLDNEAAATVLRACGANLDKLRRDLQEFIDSTTPLIPQHDDERETQPTLGFQRVLQRAVFHVQSSGKREVTGANVLVAIFSEQESQAVFLLKQQSIARIDVVNYIAHGISKVPGHAEHPQDGEQDMQDEEGGESATSNHPLDAYASNLNELARQGRIDPLVGREHEVERVAQILARRRKNNPLLVGEAGVGKTAIAEGLAKRIVDGQVPDLLADSVVYSLDLGALLAGTKYRGDFEKRFKALLNELRKRPHAVLFIDEIHTIIGAGAASGGVMDASNLLKPVLSSGEIRCIGSTTFQEFRGIFEKDRALARRFQKVDVTEPSVEDTYGILKGLKGRFEQHHHIEYSDEALRAAAELAARYINDRHMPDKAIDVIDEAGAYQRLQPEEKRVKRIEVAQVEDIVAKIARIPPKHVTTSDKELLRNLERDLKLTVFGQDDAIESLSTAIKLSRAGLKAPDKPVGSFLFAGPTGVGKTEVARQLAKALGVELVRFDMSEYMERHTVSRLIGAPPGYVGFDQGGLLTEAITKTPHCVLLLDEIEKAHPEVFNLLLQVMDHGTLTDNNGRKADFRNIILIMTTNAGAEVAARASIGFNQQDHTTDAMEVIKKSFTPEFRNRLDTIIQFGRLSTETIKSVVDKFLTELQAQLEDKRVQLEVSDAARGWLAEKGYDVQMGARPMARLIQDKIKRPLAEEILFGELAEHGGLVHVDLKGDELAFEFEITAAEPA

SEQ ID NO: 111 - protein, 101 aa, Pseudomonas aeruginosa
MIPGEYDIQPGDIELNAGRRTLALSVANTGDRPIQVGSHYHFFEVNDALAFDRPATRGMRLNIAAGTAVRFEPGQSREVELVEIGGGRRVYGFAGRVMGDL

SEQ ID NO: 112 - protein, 83 aa, Pseudomonas aeruginosa
MSLKITDDCINCDVCEPECPNGAISQGEEIYVIDPNLCTECVGHYDEPQCQQVCPVDCIPLDDANVESKDQLMEKYRKITGKA

SEQ ID NO: 113 - protein, 217 aa, Pseudomonas aeruginosa
MGLELGFRELGEVPYEPTWHAMQRFVAERDKSVMDEAWLLQHPAVFTQGQAGKAEHVLFPGDIPVIQVDRGGQVTYHGPGQLVTYLLLDVRRLGLGVRELVSRIEQSLIGLLASYDVQAVAKPDAPGVYVDGAKIASLGLRIRNGCSFHGLALNLDMDLRPFQRINPCGYAGMPMTQLRDLVGPVDFAEVCTRLRAELVSRLGYAEQKTLTGGIELT

SEQ ID NO: 114 - protein, 512 aa, Pseudomonas aeruginosa
MSQAHTPSAPLAEVYDVAVVGGGINGVGIAADAAGRGLSVFLCEQHDLAQHTSSASSKLIHGGLRYLEHYEFRLVREALAEREVLLAKAPHIVKPLRFVLPHRPHLRPAWMIRAGLFLYDHLGKREKLPASRGLRFTGSSPLKAEIRRGFEYSDCAVDDARLVVLNAISAREHGAHVHTRTRCVSARRSKGLWHLHLERSDGSLYSIRARALVNAAGPWVARFIQDDLKQKSPYGIRLIQGSHIIVPKLYEGEHAYILQNEDRRIVFAIPYLDRFTMIGTTDREYQGDPAKVAISEEETAYLLQVVNAHFKQQLAAADILHSFAGVRPLCDDESDEPSAITRDYTLSLSAGNGEPPLLSVFGGKLTTYRKLAESALTQLQPFFANLGPAWTAKAPLPGGEQMQSVEALTEQLANRYAWLDRELALRWARTYGTRVWRLLDGVNGEADLGEHLGGGLYAREVDYLCKHEWAQDAEDILWRRSKLGLFLSPSQQVRLGQYLQSEHPHRPRVHAA

SEQ ID NO: 115 - protein, 295 aa, Pseudomonas aeruginosa
MASHEHYYVPAQSKWPIIASIGLLVTVFGLGTWFNDLTAGHKESHGPWIFFVGGLIIAYMLFGWFGNVIRESRAGLYSAQMDRSFRWGMSWFIFSEVMFFAAFFGALFYVRHFAGPWLGGEGAKGVAHMLWPNFQYSWPLLQTPDPKLFPPPSAVIEPWKLPLINTILLVTSSFTVTFAHHALKKNKRGPLKAWLALTVLLGIAFLILQAEEYVHAYNELGLTLGAGIYGSTFFMLTGFHGAHVTLGALILGIMLIRILRGHFDAEHHFGFEAASWYWHFVDVVWIGLFIFVYVI

SEQ ID NO: 116 - protein, 374 aa, Pseudomonas aeruginosa
MLRHPRVWMGFLLLSAISQANAAWTVNMAPGATEVSRSVFDLHMTIFWICVVIGVLVFGAMFWSMIVHRRSTGQQPAHFHESTTVEILWTVVPFVILVVMAVPATRTLIHIYDTSEPELDVQVTGYQWKWQYKYLGQDVEYFSNLATPQDQIHNRQAKDEHYLLEVDEPLVLPVGTKVRFLITSSDVIHSWWVPAFAVKRDAIPGFVNEAWTKVDEPGIYRGQCAELCGKDHGFMPIVVDVKPKAEFDQWLAKRKEEAAKVKELTSKEWTKEELVARGDKVYHTICAACHQAEGQGMPPMFPALKGSKIVTGPKEHHLEVVFNGVPGTAMAAFGKQLNEVDLAAVITYERNAWGNDDGDMVTPKDVVAYKQKQQ

SEQ ID NO: 117 - protein, 530 aa, Pseudomonas aeruginosa
MSAVIDTPDHHAGDHHHGPAKGLMRWVLTTNHKDIGTLYLWFSFMMFLLGGSMAMVIRAELFQPGLQIVEPAFFNQMTTMHGLIMVFGAVMPAFVGLANWMIPLMIGAPDMALPRMNNFSFWLLPAAFGLLVSTLFMPGGGPNFGWTFYAPLSTTFAPHSVTFFIFAIHLAGISSIMGAINVIATILNLRAPGMTLMKMPLFVWTWLITAFLLIAVMPVLAGVVTMMLMDIHFGTSFFSAAGGGDPVLFQHVFWFFGHPEVYIMILPAFGAVSAIIPTFARKPLFGYTSMVYATASIAFLSFVVWAHHMFVVGIPVTGELFFMYATMLIAVPTGVKVFNWVTTMWEGSLTFETPMLFAVAFVILFTIGGFSGLMLAIAPADFQYHDTYFVVAHFHYVLVPGAIFGIFASAYYWLPKWTGHMYDETLGKLHFWMSFIGMNLAFFPMHFVGLAGMPRRIPDYNLQFADFNMVSSIGAFMFGTTQLLFLFIVIKCIRGGKPAPAKPWDGAEGLEWSIPSPAPYHTFSTPPEVK

SEQ ID NO: 118 - protein, 341 aa, Pseudomonas aeruginosa
MFDMMDAARLEGLHLAQDPATGLKAIIAIHSTRLGPALGGCRYLPYPNDEAAIGDAIRLAQGMSYKAALAGLEQGGGKAVIIRPPHLDNRGALFEAFGRFIESLGGRYITAVDSGTSSADMDCIAQQTRHVTSTTQAGDPSPHTALGVFAGIRASAQARLGSDDLEGLRVAVQGLGHVGYALAEQLAAVGAELLVCDLDPGRVQLAVEQLGAHPLAPEALLSTPCDILAPCGLGGVLTSQSVSQLRCAAVAGAANNQLERPEVADELEARGILYAPDYVINSGGLIYVALKHRGADPHSITAHLARIPARLTEIYAHAQADHQSPARIADRLAERILYGPQ

SEQ ID NO: 119 - protein, 141 aa, Pseudomonas aeruginosa
MFGISFSELLLVGLVALLVLGPERLPGAARTAGLWIGRLKRSFNTIKQEVEREIGADEIRRQLHNEHILSMEREAQKLLAPLTGQNPPQETPPPAAESPAPSVPTPPPTSTPAVPPADAAAPPAVAASTPPSPPSETPRNP

SEQ ID NO: 120 - protein, 347 aa, Pseudomonas aeruginosa
MKQQFERSPSESYFWPVVLAVVLHVLIFAMLFVSWAFAPELPPSKPIVQATLYQLKSKSQATTQTNQKIAGEAKKTASKQYEVEQLEQKKLEQQKLEQQKLEQQQVAAAKAAEQKKADEARKAEAQKAAEAKKADEAKKAAEAKAAEQKKQADIAKKRAEDEAKKKAAEDAKKKAAEDAKKKAAEEAKKKAAAEAAKKKAAVEAAKKKAAAAAAAARKAAEDKKARALAELLSDTTERQQALADEVGSEVTGSLDDLIVNLVSQQWRRPPSARNGMSVEVLIEMLPDGTITNASVSRSSGDKPFDSSAVAAVRNVGRIPEMQQLPRATFDSLYRQRRIIFKPEDLSL

SEQ ID NO: 121 - protein, 82 aa, Pseudomonas aeruginosa
MGIFDWKHWIVILIVVVLVFGTKRLKNLGSDVGEAIKGFRKAVNTEEDDKKDQPAAQPAQPLNQPHTIDAQAQKVEEPARKD

SEQ ID NO: 122 - protein, 176 aa, Pseudomonas aeruginosa
MQNAKLMLTCLAFAGLAALAGCSFPGVYKIDIQQGNVVTQDMIDQLRPGMTRRQVRFIMGNPLIVDTFHANRWDYLYSIQPGGGRRQQERVSLFFNDSDQLAGLNGDFMPGVSRDEAILGKEGSTTVTQPADQQKPEAQKEEPPKPGSTLEQLQREVDEAQPVPVPTPEPLDPSPQ

SEQ ID NO: 123 - protein, 421 aa, Pseudomonas aeruginosa
MSMTPIARAVAFAALGSSITVPTLAHAEFIKDSKASIELRNFYFNRDFRQEGASQSKAEEWAQGFLLRYESGYTEGTIGFGVDAIGLLGVKLDSSPDRSGTGLLKRDRETGRAQDDYGEAGITAKLRASKSTLKIGTLTPKLPVIMPNDSRLLPQTFQGGALNSMEIDGLTLDAGRLKKVNQRDSSDNEDMTITGGGKRNIVVRSGLTSDKFDFAGGSYKWTDNLSTSYHYGKLDNFYKQHYLGLVHTLPIADKQSLKSDIRWARSTDDGSSNVDNKALNAMFTYSLGYHAFGVGYQKMSGDTGFAYINGADPYLVNFIQIGDFANKDEKSWQARYDYNFAGVGIPGLTFMTRYVKGDNIDLLTTSGEGKEWERDMDIAYVFQSGPLKNLGVKWRNATMRTNYTNDYDENRLIVSYTLPLW

SEQ ID NO: 124 - protein, 145 aa, Pseudomonas aeruginosa
MDLTSKVNRLLAEFAGRIGLPSLSLDEEGMASLLFDEQVGVTLLLLAERERLLLEADVAGIDVLGEGIFRQLASFNRHWHRFDLHFGFDELTGKVQLYAQILAAQLTLECFEATLANLLDHAEFWQRLLPCDSDREAVAAVGMRV

SEQ ID NO: 125 - protein, 551 aa, Pseudomonas aeruginosa
MIARLLAALGLAAFAPALWADALTGDVQRQPLNVSAIVMFVAFVGATLCITYWASKRNRSAADYYAAGGRITGFQNGLAIAGDYMSAASFLGISALVFTSGYDGLIYSIGFLVGWPIILFLIAERLRNLGKYTFADVASYRLKQKQIRTLSACGSLVVVAFYLIAQMVGAGKLIELLFGLNYHVAVVLVGILMVLYVLFGGMLATTWVQIIKAVLLLSGASFMAIMVLKHVNFDVSTLFSEAIKVHPKGEAIMSPGGLVKDPISAFSLGFALMFGTAGLPHILMRFFTVSDAKEARKSVFYATGFIGYFYILTFIIGFGAILLVSTNPDFKDATGALIGGNNMAAVHLADAVGGSLFLGFISAVAFATILAVVAGLTLAGASAVSHDLYASVFKGGKANEKDELRVSKMTTVALGVVAIVLGILFEKQNIAFMVGLAFSIAASCNFPVLLLSMYWKKLTTRGAMIGGWMGLITAVGLMVLGPTIWVQILGHEKAIYPYEYPALFSMIVAFVGIWFFSITDKSAAADEERARFFPQFIRSQTGLGASGAVAH

SEQ ID NO: 126 - protein, 305 aa, Pseudomonas aeruginosa
MNYPVDHLTALKVFRAVAANGGFAAAARQMNLSPAAVSKNVAELEAHLKVRLINRTTRSMSLTEAGEVYRQRLERILDDLEAADAALTSMQQGPSGLLRVSAPLTLALTCLTPAIPAFLQRYPELRLELLLQDGRQDLIAEGIDLALRGSDRVADSGLVARPLLVLEHVLCAAPAYLSQHGQPLRPEALREHECIRFSLSGHADRWTFRKDRECIAVPIAGRYRVSSSLAVRDALLAGFGLSLIPRLYVQAELAEGRLVELLADWKADETAIHAVYPSRQLAGKTRVFLDFLTETMAQGHDSPTL

SEQ ID NO: 127 - protein, 284 aa, Pseudomonas aeruginosa
MTTSLQPVHALVPGANLEAYVHSVNSIPLLSPEQERELAERLFYQQDLEAARQMVLAHLRFVVHIAKSYSGYGLAQADLIQEGNVGLMKAVKRFNPEMGVRLVSFAVHWIKAEIHEFILRNWRIVKVATTKAQRKLFFNLRSQKKRLAWLNNEEVHRVAESLGVEPREVREMESRLTGQDMAFDPAADADDESAYQSPAHYLEDHRYDPARQLEDADWSDSSSANLHEALEGLDERSRDILQQRWLSEEKATLHDLAEKYNVSAERIRQLEKNAMSKLKGRILA

SEQ ID NO: 128 - protein, 334 aa, Pseudomonas aeruginosa
MALKKEGPEFDHDDEVLLLEPGIMLDESSADEQPSPRATPKATTSFSSKQHKHIDYTRALDATQLYLNEIGFSPLLTPEEEVHFARLAQKGDPAGRKRMIESNLRLVVKIARRYVNRGLSLLDLIEEGNLGLIRAVEKFDPERGFRFSTYATWWIRQTIERAIMNQTRTIRLPIHVVKELNVYLRAARELTHKLDHEPSPEEIANLLEKPVAEVKRMLGLNERVTSVDVSLGPDSDKTLLDTLTDDRPTDPCELLQDDDLSESIDQWLTELTDKQREVVIRRFGLRGHESSTLEEVGQEIGLTRERVRQIQVEALKRLREILEKNGLSSDALFQ

SEQ ID NO: 129 - protein, 275 aa, Pseudomonas aeruginosa
MDKPASRHFSVLIIDDEPQVTSELRELLENSGYRCVTSTHRESAIASFQADPNIGLVICDLYLGQDNGIRLIESLKEVAGNGRFFESIILTGHDGRQEVIEAMRVGAADYYQKPVAPQELLHGLERLESRLHERVRSQLSLSHVNQRLEYLAESLNSIYRDIHKIKYEVHGNSQPSALRSEDSQPSAPPAPVAESQVSPSNPLFGKLSPRQQAVARLVSKGLTNYQIAYELGITENTVKLYVSQVLRLMHMHNRTQLALALSPAAMQQGSGAVVH

SEQ ID NO: 130 - protein, 212 aa, Pseudomonas aeruginosa
MPSVSDPRALARLLELILSTAQAGLAFSKDRFDIGRFRALQHAVAEFIASDQGVAYERVENWIALDSHYPTPKLDVRALILDSQQRVLLVREASDSRWTLPGGWCDVNESPADAVVRETQEESGLEVRAIRLLALLDKHKHPHPPQLPHALKAFFFCHVTGGSLQQQTDETSAAEYFTVDALPPLSEHRVLASQIQTLWQRIHAETPEALFD

SEQ ID NO: 131 - protein, 71 aa, Pseudomonas aeruginosa
MPAVKVKENEPFDVALRRFKRSCEKAGVLAEVRSREFYEKPTAERKRKAAAAVKRHAKKVQREQRRRERLY

SEQ ID NO: 132 - protein, 178 aa, Pseudomonas aeruginosa
MLNGPIPPHIDPRKLVDRGATLEGVYALADMPRVCEQLTSDAGEVRVKVSFERDHQKLAVMHMQLDTEVSMVCQRCLDAAAIPVHGEYTYAILREGQSADGLPKGYDALEVGEEPLDLLALVEDELLLALPIVPAHDPEVCQHPAGFVVEDEPESSEVEDKRPNPFSVLAQLKRDPNV

SEQ ID NO: 133 - protein, 158 aa, Pseudomonas aeruginosa
MRTSFFAAAALLAVSAFAQAHEYNAGQLHIEHPWALALPPTSPNGAAYFVVQNHGKENDTLLGADTPRAASAEVHEHVHKNGMMSMQKVDSVDVAPGKDLRFAPGGYHLMLMGLKQPLVAGERFPLTLHFRKAGDVPVEIVVESKAPAEQGGHEQHGH

SEQ ID NO: 134 - protein, 103 aa, Pseudomonas aeruginosa
MNDSIYQRIDTNPRFKELVAKRERFAWILSSIMLGLYVIFILLIAFQPQLLGARISPDSSVTWGIPMGVGLILAAFILTGLYVRRANGEFDSLNQEILKEAQQ

SEQ ID NO: 135 - protein, 65 aa, Pseudomonas aeruginosa
MNSDVIKGKWKQLTGKIKERWGDLTDDDLQAADGHAEYLVGKLQERYGWSKERAEQEVRDFSDRL

SEQ ID NO: 136 - protein, 78 aa, Pseudomonas aeruginosa
MVLFNDDYTPMDFVVEVLEVFFNMDREKATKIMLTVHTQGKAVCGLFTRDVAETKAMQVNQYARESQHPLLCEIEKDS

SEQ ID NO: 137 - protein, 184 aa, Pseudomonas aeruginosa
MSDAKVDTRRLVGRLLLVTVLMFAFGFALVPLYDVMCRALGINGKTAGSAYSGEQQVDVGREVKVQFMTSNNIDMVWEFRSAGDQLVVHPGAVNQMVFYARNPSDKPMTAQAIPSIAPAEAAAYFHKTECFCFTQQVLQPGESIEMPVRFIVDRDLPKDVRHVTLAYTLFDITARKPPVPVAGR

SEQ ID NO: 138 - protein, 640 aa, Pseudomonas aeruginosa
MSIFSHFQERFEATRQEEYSLQEYLDLCKQDKTAYASAAERLLMAIGEPELLDTSVDSRLSRIFSNKVIRRYPAFADFHGMEECIDQIVAFFRHAAQGLEEKKQILYLLGPVGGGKSSLAEKLKQLMEKVPFYAIKGSPVFESPLGLFNPDEDGAILEEDYGIPRRYLRSIMSPWATKRLNEFGGDISQFRVVKLYPSILNQIAIAKTEPGDENNQDISALVGKVDIRKLEEYPQNDADAYSYSGALCRANQGLMEFVEMFKAPIKVLHPLLTATQEGNYNSTEGLGALPYSGIILAHSNESEWHSFRNNKNNEAFIDRIYIVKVPYCLRVADEIKIYDKLLVNSSLAHAHCAPDTLKMLSQFSVLSRLKEPENSNIYSKMRVYDGENLKDTDPKAKSIQEYRDSAGVDEGMAGLSTRFAFKILSKVFNFDPHEVAANPVHLLYVLEQQIEQEQFQPETRERYLRFIKEYLAPRYVEFIGKEIQTAYLESYSEYGQNIFDRYVLYADFWIQDQEYRDPETGEILNRAALNEELEKIEKPAGISNPKDFRNEIVNFVLRARAGNNGKNPSWLSYEKLRVVIEKKMFSNTEDLLPVISFNAKASKEDQQKHNDFVKRMVERGYTEKQVRLLSEWYLRVRKSQ

SEQ ID NO: 139 - protein, 108 aa, Pseudomonas aeruginosa
MMKGGMAGLMKQAQQMQEKMQKMQEELANAEVTGQSGAGLVSVVMTGRHDVKRVSLDDSLMQEDKEILEDLIAAAVNDAVRKIEQNNQEKMSGFTSGMQLPPGFKMPF

SEQ ID NO: 140 - protein, 423 aa, Pseudomonas aeruginosa
MSYVIDRRLNGKNKSTVNRQRFLRRYREHIKKAVEEAVSRRSITDMEHGEQISIPGRDIDEPVLHHGRGGRQTVVHPGNKEFTAGEHIARPSGGGGGRGGGKASNSGEGMDDFVFQITQEEFLDFMFEDLELPNLVKRHITGTDTFKTVRAGISNDGNPSRINIVRTLRSAHARRIALSGGSRAKLRAALKELERIKREEPDNLGDIQELELEIAKLRARIDRVPFLDTFDLKYNLLVKQPNPTSKAVMFCLMDVSGSMTQATKDIAKRFFILLYLFLKRNYEKIEVVFIRHHTSAREVDEEEFFYSRETGGTIVSSALKMMQEIMAERYPTHEWNIYAAQASDGDNWNDDSPVCRDILLKQIMPFVQYYTYVEITPREHQALWFEYERVREAFEDSFAQQQIVSASDIYPVFRELFQRRLVA

SEQ ID NO: 141 - protein, 141 aa, Pseudomonas aeruginosa
MATRRKTTPQEIDDIQDRMGSMRELDFDERRQARKARIGDERPEAEVEAEFSSRRVREAGHAGGQPDEDDGYQDNVGMDDLAPETLIDESGARSPAERGGESPADKRLRVVHGNEIGAGHGLDEAELARRDPLDGSSDEER

SEQ ID NO: 142 - protein, 128 aa, Pseudomonas aeruginosa
MGDRLAVPIRYYALAGIAAAILLNVLLRGVVRFGGLPASLLIAALVAGGLAWWFARAQRRWPTWGERLRLVALYGGVLGVLYLLLVGLASLKGDPSPAALLIVVLHYLCYPALLLVFFSGRVYGFFLR

SEQ ID NO: 143 - protein, 52 aa, Pseudomonas aeruginosa
MSLGTILLIILILLLIGGLPVFPHSRNWGYGPSGIIGALLVVLLVLLLLGMI

SEQ ID NO: 144 - protein, 158 aa, Pseudomonas aeruginosa
MLKLLVVVASLFIGGGMAMGEPGGADGFSAVAAVDPGANPEAQLDEEPQEVSEAPAYRVDDLTFLYLTHEVYLEPYVSCRPKVLGERSYVACWNETYSGRSPLNFWEYDGGDFLALNDPARVLAEGKFASEQRIGEAPLPLPLDIDLDQLERAYSLMM

SEQ ID NO: 145 - protein, 711 aa, Pseudomonas aeruginosa
MSIDILVTTDRLNKDPLGREFISYIESNSDRLNLAGSALYYDFPAYSDYDTVTHKPDVLILSPAHGILAIRLINLARLNQGALSQADESLNQFCSILIGRLLKSKPLREGRSKLSFEVTPVIYCDQKTDVLPEDLDCELVSSYGAFEALIEKLKDRESNAETLSEIRSVLEGAKALTRASKRIVEDPEVTPAAAALAKLESEIANFDQKQRRAALVTIDGPQRIRGLAGSGKTVILAMKAAHLHMTRPNDKILVTFFTKSLRSPIKDLVTKFYRHYKEIDPDWNNIHIRHGWGGSNSSGTYADACRRSGRMPRSYSSARDAAPRGVEPFSFACEELINSAQSLEYYDHILIDEGQDFPDGFYKLCFELAKGDRDKKNIVWAYDELQNILNVKMRSPAELFGQDDDGEPRISLARAEKNLPPGATNDTVLSKCYRNQREVLVAAHALGFGIYSNIVQLLESPDHWRDVGYDVLTPDFQVGNDIEILRPVENSPVALDTDGLPPLIGNFIASSFEKEISWITKEIINFLDMGLLPEDIIVVALDDRHMRNYFRYLSESLASHEISVNNIHADPYSEPPFSISGKITLSTVYRAKGNEAAVVFALGVDALSLRLRADRNKIFAAFTRTKAWLRVSGLGDSARRVAVEIDTALRNFPHLKFKMPDISEIDLIQRDLSKRSIKAKKIRNEYIQKLKDEGFTEDEIADILSLEEKDE

SEQ ID NO: 146 - protein, 72 aa, Pseudomonas aeruginosa
MQDLGFALLLIGYVWSVASGGRRSIPCALLCLLLFPLAQLAFAINDAPMRPPLALAAFGAGLAYLGGGSVFG

SEQ ID NO: 147 - protein, 106 aa, Pseudomonas aeruginosa
MKLSDSFDARRLRPRRPRVWRWRLAAAFAALVASLGVLLSLAGTSALIGRQPAVGMLHIEPAAGGVLLALGLLSLCLGVFFWRFSRRRSRRNSDLSLSPNLMKKRD

SEQ ID NO: 148 - protein, 104 aa, Pseudomonas aeruginosa
MTTVADIVSTMKSKFNASAAAGLDLVFQFNIEDGDNHYLVVKDGTCEVVQGDAENPNVTLIMDSETLKGITSGETDGMQAFMAGKLRAEGDMMLAMKLGELFPV

SEQ ID NO: 149 - protein, 95 aa, Pseudomonas aeruginosa
MPGFFMRSGNSGGLVERVRLADADAFVGLRQVEVFMAAGGAVHQPAVVLDIEGKIDDHGLDLVQVVDQIVMFVGCGFQHDDSPVLSFAWVGFCDR

SEQ ID NO: 150 - protein, 113 aa, Pseudomonas aeruginosa
MTLRNGVPSMTKDEKEKTHVDAIIERYKDLMVEIPPADRQPGLSLLWPVPAQPAIDKGVRQAENWLADQIEGQLWTAFAFGRDSLPTPMQKTAFEVAFLTRLQQRLVAARRSG

SEQ ID NO: 151 - protein, 73 aa, Pseudomonas aeruginosa
MLLSLLCLSTLALGLALSLAGSTREEREQAALLPFADDPEAARRVARDTGKICRQVVRPLEESREAAGPPFLA

SEQ ID NO: 152 - protein, 168 aa, Pseudomonas aeruginosa
MDKRLLSKGLVLGLLSLGSMTAHADAAGGNGCGWGNMVFEGQRGLFPHLLATTTNGTSGNATFGMTSGTNGCNTNARLGYGGRSIFAMNGMLDNIAEDMAKGQGEALDAYAVLLGVEAKDRAHFAQVTQQHFGEIFASKDATGEQVLSNTLAVMSRDGTLARYAKQPA

SEQ ID NO: 153 - protein, 173 aa, Pseudomonas aeruginosa
MKKLLPLAVLAALSSVHVASAQAADVSAAVGATGQSGMTYRLGLSWDWDKSWWQTSTGRLTGYWDAGYTYWEGGDEGAGKHSLSFAPVFVYEFAGDSIKPFIEAGIGVAAFSGTRVGDQNLGSSLNFEDRIGAGLKFANGQSVGVRAIHYSNAGLKQPNDGIESYSLFYKIPI

SEQ ID NO: 154 - protein, 455 aa, Pseudomonas aeruginosa
MKTTKILLHTGVLALSLLATQVMAAVSADEAAKLGTSLTPLGAEKAGNADGSIPAWDGGLATNAGSVDSRGFLANPYASEQPLFTITAQNVDQYKDKLTPGQLAMFKRYPDTYKIPVYKTHRSATVPAAVQEAAKRNATTTKLVEGGNGLENFDTANPFPIPQNGLEVIWNHITRYRGGSVRRLVTQATPQVNGSYQLVYFQDAFTFRTNLKDYNPNKPSNVLFYFKQRVTAPSRLAGNVLLVHETLNQVKEPRLAWLYNAGQRRVRRAPQVSYDGPGTAADGLRTSDNFDMYNGAPDRYDWKLEGKKEIYIPYNSYKLDDPKIKYSEIVKAGHINQDLTRYELHRVWHVVATLKPGERHIYAKRDFYIDEDTWQAAEIDHYDGRGTLWRVAEAHAEQYYDKQVPWYAVETLYDLLSGRYLALGMKNEEKQAYDFNYSASESDYTPAALRQEGVR

SEQ ID NO: 155 - protein, 330 aa, Pseudomonas aeruginosa
MLAVLSVSLMVVDARFDYLEPVRSKLGMVLTPFYGLAEMPVRAWEGVRDQFSSRSELIAENERLKAESLLMQRRVQKLAALTEQNVRLRELLNSAALVDDKVLVSELIGVDPNPFTQRIMIDKGENDGVFVGQPVLDASGLMGQVVEVMPYTARVLLLTDTTHSIPVQVNRNGLRAIAVGTGNPERLELRYVADTADIKEGDLLVSSGLGQRFPAGYPVATVKEVIHDSGQPFAVVRAVPTAKMNRSRYVLLVFSDSRTPEQRANDAAEAQEEADKKAAAGAQAPQPAAQPAAAPSPATPAAQGAAQQPAAAPAPAPTQPAAPAANGGRR

SEQ ID NO: 156 - protein, 90 aa, Pseudomonas aeruginosa
MRKPELAAAIAEKADLTKEQANRVLNALLDEITGALNRKDSVTLVGFGTFLQRHRGARTGKNPQTGQPVKIKASNTVAFKPGKALRDAVN

SEQ ID NO: 157 - protein, 716 aa, Pseudomonas aeruginosa
MSRKRSPAPSRISEGHPFPLGATWDGLGVNFALFSAHATKVELCLFDARGEKEIERIELPEYTDEIWHGYLPDAHPGQIYGYRVHGPYEPDAGHRFNPNKLLLDPYAKQLVGRLRWSEALFGYTIGSADADLSFDERDSAPFVPKSKVIDPAFTWAERPPVRVPWDRTVIYEAHLRGLSMRHPQVPEAVRGTFAGLMNADLLAHIRRLGVTSVELLPIHGFVDDKHLLENGMSNYWGYNSIAFFAPHPAYLASGQVNEFKEMVAHLHDAGLELILDVVYNHTAEGNELGPTLCMRGIDNASYYRLMPDQRRYYINDSGTGNTLDLSHPCVLQMVTDSLRYWATEMRVDGFRFDLATILGRHPDGFDERHGFLVACRQDPVLSKCKLIAEPWDCGPGGYQVGGFPPGWAEWNDRFRDCVRAYWRGDDGMLPELARRLTASGDLYDQRGRRPYASVNFVTAHDGFTLRDVVSYDHKHNEANGENNADGSDHNLSWNHGCEGPTDDPEIRALRLRQMRNLLSTLLLSQGTPMLVAGDEFSRTQQGNNNVYCQDNELGWIDWRLDDEGRSLLAFTQRLLALRQRYPILRRGRFLVGEYNEALGVKDVTWLAPGGEEMTEEHWHDEHARCLGVLLDGRAQPTGILRSGEDATLLLILNAYHDAVSFRLPEVAEGSGWTCLLDTQRPEDPLGERYPFASEFLVGGRSFLLFELQPPGAGAEG

SEQ ID NO: 158 - protein, 173 aa, Pseudomonas aeruginosa
MPHSYRKMESPVGTLTLVARDDAFLVAILWQHERPNRVPLDEMRLSEDSSLLAETERQLREYFSGKRSRFELPLDFQGTEFQKKVWSALLTIPFGETRSYTEIAVQIGSPNAVRAVGAANGRNPLSIVAPCHRVIGASGGLTGFAGGLAAKQWLLRLETRGRTPDLLSMIEDE

SEQ ID NO: 159 - protein, 127 aa, Pseudomonas aeruginosa
MPMRSLIVACLALSATGCNSWSLNSDLNGAYRAYDKGDCAQVMLDLSRAERRIRARPYLQPEISLLRGQCLERQSLFVDAAQTYHFIIARYPTSEYAYRAKARLETLRQLGRLSETPASASAVPTRL

SEQ ID NO: 160 - protein, 77 aa, Pseudomonas aeruginosa
MNLKPQTLMVAIQCVAARTRELDAQLQNDDPQNAAELEQLLVGYDLAADDLKNAYEQALGQYSGLPPYDRLIEEPAS

SEQ ID NO: 161 - protein, 236 aa, Pseudomonas aeruginosa
MTDPIRLSKRLAELTACSRREAELYIEGGWVSVDGEVIEEPQFKVLDQRVELLPGARAETIEPATLLLHKPAGWRHDDFEGLLAAGRRWSDDPSPLRALKKHFARQRPTLALDSEASGLVVFSQQHGVLRKLVDDGARLEQEYLVEVAGDLAAGGLERLRHGLAYQGRRLSPCKVSWQNESHLRFALKDVLPGQLRFMCEREGLEVRSIRRLRIGALSLARLPLGEWRYLGLHERF

SEQ ID NO: 162 - protein, 76 aa, Pseudomonas aeruginosa
MLIPHDLLEADTLNNLLEDFVTREGTDNGDETPLDVRVERARHALRRGEAVILFDPESQQCQLMLRSEVPAELLRD

SEQ ID NO: 163 - protein, 97 aa, Pseudomonas aeruginosa
MKLRPLHDRVVIRRSEEETKTAGGIVLPGSAAEKPNRGEVVAVGTGRVLDNGEVRALAVKVGDKVVFGPYSGSNAIKVDGEELLVMGESEILAVLED

SEQ ID NO: 164 - protein, 122 aa, Pseudomonas aeruginosa
MRSWIYLLLAIGAEVIGTTSMKLAATHAPVAGMLLMYGMIGLSYFFLALAVKRVPVGVAYALWEGIGIVLITAVSVAWLGESIGLYKAVGLGVMIAGILLIKSGTRNASGTPAQSRGEAVTC

SEQ ID NO: 165 - protein, 792 aa, Pseudomonas aeruginosa
MNSATLTELDLPVSGMTCASCAGRVERALKKVPGVAAASVNLASEQARVQAPADSLPALVAAVEQAGYQVPARSLELSIEGMTCASCVGRVERALKKVPGVREVSVNLASERAHLELLGAVDSQALLQAVEQAGYKARLLDAGQPRQDDAERRLRRERWWVIAALLLALPLVLPMLVEWAGLHWMLPPWAQFLLATPVQFVIGARFYVSAWRAVKAGAGNMDLLVALGTSAGYGLSVYLWLTAPPGHMPHLYFEASTVVIALILLGKYLESRAKRQTASAIRALEALRPERAVRLRDGREEEVAIAELRLGDEVVVRPGERFPVDGEVLDGSSHADEALITGESLPVPKAPGDKVTGGAINGEGRLLLRTTALGGETVLAKIIRLVEDAQAAKAPIQKLVDKVSQVFVPVVILIALVTLGAWLVAGVGLEQALVNAVAVLVIACPCALGLATPTAIMAGTGVAARHGILIKDAESLEVAHAVTSVAFDKTGTLTSGRPQIIHLGGDDQEQLLRLAGALQRGSEHPLAKAVLERCAERDLEVPPVNASQALSGRGIQGEVEGRRLALGNRRLLDEQELKPGALASAAADWEAEGRTLSWLLELAPEKRVLGLFAFGDSLKDGAAEAVEALRGRDIHSHLITGDNRGSAAVVAKALGIDDVHAEVLPADKAATVAELKGRGRVVAMVGDGINDAPALAAADVGIAMGGGTDVAMHAAGITLMRGDPRLVPAALDISRRTYAKIRQNLFWAFIYNVIGIPLAAFGLLNPMVAGAAMAFSSVSVVGNALLLRRWKP

SEQ ID NO: 166 - protein, 212 aa, Pseudomonas aeruginosa
MRRTKEDSEKTRTAILLAAEELFLEKGVSHTSLEQIARAAGVTRGAVYWHFQNKAHLFNEMLNQVRLPPEQLTERLSGCDGSDPLRSLYDLCLEAVQSLLTQEKKRRILTILMQRCEFTEELREAQERNNAFVQMFIELCEQLFARDECRVRLHPGMTPRIASRALHALILGLFNDWLRDPRLFDPDTDAEHLLEPMFRGLVRDWGQASSAP

SEQ ID NO: 167 - protein, 210 aa, Pseudomonas aeruginosa
MLVCAAFSGGAGASDAGALLEAARRGDTAQVASLLERKVEVDAPSADGSTPLLLATANDHLAVARQLIEAGADVNRQNSRLDSPYLLAGAEGRLEILRLTLLHGADLKSTNRYGGTALIPACERGHVEVVKTLLQAGVDPNHVNKLGWTGLLEAILLSDGGPRHQEIVRLLIDAGADVNLADADGVSPLAHARQRGQGGIERLLLAAGAQ

SEQ ID NO: 168 - protein, 637 aa, Pseudomonas aeruginosa
MGKIIGIDLGTTNSCVAILENGNVKVIENAEGARTTPSIIAYTNDGETLVGQPAKRQAVTNPQNTLYAVKRLIGRRFEENVVQKDIQMVPYSIVKADNGDAWVEVKGQKMAPPQISAEVLKKMKKTAEDYLGEPVTEAVITVPAYFNDSQRQATKDAGRIAGLDVKRIINEPTAAALAYGLDKAKGDHTVIVYDLGGGTFDVSVIEIAEVDGEHQFEVLATNGDTFLGGEDFDIRLIDYLVDEFKKESGINLKGDPLAMQRLKEAAEKAKIELSSTQQTDVNLPYVTADASGPKHLNVKVSRAKLESLVEDLVQRTIEPCRTALKDAGLDVSDIHEVILVGGQTRMPLVQKTVAEFFGKEARKDVNPDEAVAVGAAIQGAVLAGDVKDVLLLDVTPLTLGIETLGGVMTGLIEKNTTIPTKKSQVFSTADDNQGAVTIHVLQGERKQAAQNKSLGKFDLADIPPAPRGVPQIEVTFDIDANGILHVSAKDKATGKQQSIVIKASSGLSEDEIQQMVRDAEANAEEDRKFEELAAARNQGDALVHATRKMITEAGDKATAEDKATIEKALGELEAAVKGDDKAEIEAKMNALSQASTPLAQKMYAEQAQQGEDAPQGEQAKAADDVVDAEFEEVKDNK

SEQ ID NO: 169 - protein, 182 aa, Pseudomonas aeruginosa
MNKSMLVGAVLGAVGVTAGGAVATYSLVDRGPDYAEVVAVQPVKETIKTPRQVCKDVAVTRQRPVKDQHQIAGTAIGAVVGGLLGNQIGGGTGKKIATVAGAVGGGYAGNKVQEGMQERDTYTTTETRCSTVHDSSEKVVGYDVKYMLDGKAGQIRMERDPGSQIPVDKNGRLILSQGETLR

SEQ ID NO: 170 - protein, 809 aa, Pseudomonas aeruginosa
MSKNARYAWRLSLGGLLLGLLACAVYLLAVPLGFKYGEIQVGHGLEHEGRIAWDAAGVPHIRAQSLEDGYFLLGYSHARDRLWQMEFARRYAGGTLSEVFGAKTLPMDRFARTLGFRRTAEGIYANLDAPTRVLLQRYSDGINAYLELAPAALPLEFSLVRHERPGPWGPVDSLSLHLLYSWTLSANLGMQLQRLALAEHLDLARINEVFAPYPGERPPATRDYASLYRSLHGTPDAGKLLGQLPGSNVEGIGSNNWVVSASRSATGKPLLANDPHLRLTNPAAFYLASLKIPGLSLTGANFAGAPLFVIGHNQRIAWGYTNTGSHIQDAYLERVDPQDPRRYLTPDGYRPFETRLERIAVRDGETVSLEVRSTRHGPVISDIYEPARLPQAQRDRLVIALAWTGLDRHDKTFPSLLAINRAEGWEQFLDAAANFGVPPQNMVYADVEGNIGYVSAGRVPLRGADDDLHGLAPSPGWESRYDWVGYVPESAKPRSLNPREGFIATANQRIVPPDNAFDFGHDWVLPYRYDRIREWLGGPGQRTLEDSLELQNDEFSSVMASLLPKMLEQVSDPELRASEAFALLQGWNHQAAADLAAPLIAGYWVRAFTRELLQPRIGTQLLASGWNQRNYDGFLRLILDGQADLRFWCGQEQGCDLKLNQSLRRALDELRAAHGSAPSGWKWGEAHAALAEHVPFHKTPLRALFDLKNNKGGDNFSVNVGRFDYSDPANPFNTRIAATLRMVIDLADFDNSRYALSTRNSGLPFDGATDLNELWARGAYIRIADDAPDATDRQLVLRPSASSSGEPRP

(19) United States
(12) Patent Application Publication
(10) Pub. No.: US 2003/0113742 A1
(43) Pub. Date: Jun. 19, 2003

(54) Methods and compositions for the modulation of biofilm formation

(75) Inventors: Marvin Whiteley, Coralville, IA (US); M. Gita Bangera, Lynnwood, WA (US); Stephen Lory, Cambridge, MA (US); Everett Peter Greenberg, Iowa City, IA (US)

Correspondence Address: LAHIVE & COCKFIELD, 28 STATE STREET, BOSTON, MA, 02109, US

(73) Assignee: University of Iowa Research Foundation, Iowa City, IA (US)

(21) Appl. No.: 10/127,032

(22) Filed: Apr. 19, 2002

Related U.S. Application Data
(60) Provisional application No. 60/285,190, filed on Apr. 20, 2001.
(60) Provisional application No. 60/344,142, filed on Oct. 24, 2001.

(51) Int. Cl. C12Q 1/68; G01N 33/554; G01N 33/569; A61K 39/40; A61K 31/704

(52) U.S. Cl. 435/6; 514/35; 435/7.32; 424/170.1

(57) ABSTRACT

The present invention relates to methods for the modulation of biofilm formation and antibiotic resistance. Specifically, the present invention identifies the differential expression of biofilm-associated genes in biofilms, relative to their expression in non-biofilm producing bacterial cells. The present invention also identifies the differential expression of biofilm-associated genes in biofilms treated with antibiotic, relative to their expression in untreated biofilms. The present invention describes methods for the diagnostic evaluation of biofilm formation. The invention also provides methods for identifying a compound capable of modulating biofilm formation and antibiotic resistance. The present invention also provides methods for the identification and therapeutic use of compounds as treatments of biofilm-associated diseases or disorders.

### RELATED APPLICATIONS

This application claims priority to U.S. Provisional Patent Application No. 60/285,190, filed on Apr. 20, 2001, and to U.S. Provisional Patent Application No. 60/344,142, filed on Oct. 24, 2001, the contents of which are incorporated herein in their entirety by reference.

### GOVERNMENT FUNDING

Work described herein was supported by funding from the National Institute of Health Grant GM 59026. The U.S. Government therefore may have certain rights in this invention.

### BACKGROUND OF THE INVENTION

Biofilms are defined as an association of microorganisms, single or multiple species, that grow attached to a surface and produce a slime layer that provides a protective environment (Costerton, J. W. (1995) J Ind Microbiol. 15(3):137-40, Costerton, J. W. et al. (1995) Annu Rev Microbiol. 49:711-45). Biofilms are structured communities of cells embedded in an extracellular polysaccharide (EPS) matrix (J. W. Costerton, et al. Ann. Rev. Microbiol. 49, 711 (1995); D. DeBeer, et al. Biotech. Bioeng. 44, 636 (1994); J. R. Lawrence, et al. J. Bacteriol. 173, 6558 (1991)). Typically, biofilms produce large amounts of extracellular polysaccharides, responsible for the slimy appearance, and are characterized by an increased resistance to antibiotics (1000-to 1500-fold less susceptible). Bacteria growing in biofilms possess characteristics distinct from their free-floating or swimming (planktonic) counterparts. Of particular importance, biofilm bacteria are resistant to antimicrobial treatments, and to host immune defenses (J. W. Costerton, et al. Ann. Rev. Microbiol. 49, 711 (1995); D. DeBeer, et al. Biotech. Bioeng. 44, 636 (1994); J. R. Lawrence, et al. J. Bacteriol. 173, 6558 (1991); J. W. Costerton, P. S. Stewart, E. P. Greenberg, Science 284, 1318 (1999)).

Several mechanisms are proposed to explain this biofilm resistance to antimicrobial agents (Costerton, J. W. et al. (1999) Science. 284(5418):1318-22). One idea is that the extracellular matrix in which the bacterial cells are embedded provides a barrier toward penetration by the biocides. A further possibility is that a majority of the cells in a biofilm are in a slow-growing, nutrient-starved state, and therefore not as susceptible to the effects of anti-microbial agents. A third mechanism of resistance could be that the cells in a biofilm adopt a distinct and protected biofilm phenotype, e.g., by elevated expression of drug-efflux pumps.

In most natural settings, bacteria grow predominantly in biofilms. Biofilms of P. aeruginosa have been isolated from medical implants, such as indwelling urethral, venous or peritoneal catheters (Stickler, D. J. et al. (1998) Appl Environ Microbiol. 64(9):3486-90). Chronic P. aeruginosa infections in cystic fibrosis lungs are considered to be biofilms (Costerton, J. W. et al (1999) Science. 284(5418):1318-22).

In industrial settings, the formation of biofilms is often referred to as ‘biofouling’. Biological fouling of surfaces is common and leads to material degradation, product contamination, mechanical blockage, and impedance of heat transfer in water-processing systems. Biofilms are also the primary cause of biological contamination of drinking water distribution systems, due to growth on filtration devices.

P. aeruginosa is a soil and water bacterium that can infect animal hosts. Normally, the host defense system is adequate to prevent infection. However, in immunocompromised individuals (such as burn patients, patients with cystic fibrosis, or patients undergoing immunosuppressive therapy), P. aeruginosa is an opportunistic pathogen, and infection with P. aeruginosa can be fatal (Govan, J. R. et al. (1996) Microbiol Rev. 60(3):539-74; Van Delden, C. et al. (1998) Emerg Infect Dis. 4(4):551-60).

For example, cystic fibrosis (CF), the most common inherited lethal disorder in Caucasian populations (˜1 out of 2,500 life births), is characterized by bacterial colonization and chronic infections of the lungs. The most prominent bacterium in these infections is P. aeruginosa—by their mid-twenties, over 80% of people with CF have P. aeruginosa in their lungs (Govan, J. R. et al. (1996) Microbiol Rev. 60(3):539-74). Although these infections can be controlled for many years by antibiotics, ultimately they “progress to mucoidy,” meaning that the P. aeruginosa forms a biofilm that is resistant to antibiotic treatment. At this point the prognosis is poor. Once CF lungs have been colonized, P. aeruginosa cannot be eradicated by even the most aggressive antibiotic therapies (J. W. Costerton, et al. Science 284, 1318 (1999); N. Hoiby, Ann. Rev. Med. 44, 1 (1993); J. L. Burns, et al. Adv Pediatr. Infect. Dis. 8, 53 (1993); Singh, et al, Nature 407, 762 (2000)). The median survival age for people with CF is the late 20s, with P. aeruginosa being the leading cause of death (Govan, J. R. et al. (1996) Microbiol Rev. 60(3):539-74). According to the Cystic Fibrosis Foundation, treatment of CF cost more than $900 million in 1995 (Cystic Fibrosis Foundation website).

P. aeruginosa is also one of several opportunistic pathogens that infect people with AIDS, and is the main cause of bacteremia (bacterial infection of the blood) and pneumonitis in these patients (Rolston, K. V. et al. (1990) Cancer Detect Prev. 14(3):377-81; Witt, D. J. et al. (1987) Am J Med. 82(5):900-6). A recent study of 1,635 AIDS patients admitted to a French hospital between 1991-1995 documented 41 cases of severe P. aeruginosa infection (Meynard, J. L. et al. (1999) J Infect. 38(3):176-81). Seventeen of these had bacteremia, which was lethal in 8 cases. Similar, numbers were obtained in a smaller study in a New York hospital, where the mortality rate for AIDS patients admitted with P. aeruginosa bacteremia was about 50% (Mendelson, M. H. et al. 1994. Clin Infect Dis. 18(6):886-95).

In addition, about two million Americans suffer serious bums each year, and 10,000-12,000 die from their injuries. The leading cause of death is infection (Lee, J. J. et al. (1990) J Burn Care Rehabil. 11(6):575-80). P. aeruginosa bacteremia occurs in 10% of seriously burned patients, with a mortality rate of 80% (Mayhall, C. G. (1993) p. 614-664, Prevention and control of nosocomial infections. Williams & Wilkins, Baltimore; McManus, A. T et al. (1985) Eur J Clin Microbiol. 4(2):219-23).

Such infections are often acquired in hospitals (“nosocomial infections”) when susceptible patients come into contact with other patients, hospital staff, or equipment. In 1995 there were approximately 2 million incidents of nosocomial infections in the U.S., resulting in 88,000 deaths and an estimated cost of $ 4.5 billion (Weinstein, R. A. (1998) Emerg Infect Dis. 4(3):416-20). Of the AIDS patients mentioned above who died of P. aeruginosa bacteremia, more than half acquired these infections in hospitals (Meynard, J. L. et al. (1999) J Infect. 38(3):176-81).

Nosocomial infections are especially common in patients in intensive care units as these people often have weakened immune systems and are frequently on ventilators and/or catheters. Catheter-associated urinary tract infections are the most common nosocomial infection (Richards, M. J. et al (1999) Crit Care Med. 27(5):887-92) (31% of the total), and P. aeruginosa is highly associated with biofilm growth and catheter obstruction. While the catheter is in place, these infections are difficult to eliminate (Stickler, D. J. et al. (1998) Appl Environ Microbiol. 64(9):3486-90). The second most frequent nosocomial infection is pneumonia, with P. aeruginosa the cause of infection in 21% of the reported cases (Richards, M. J. et al. (1999) Crit Care Med. 27(5):887-92). The annual costs for diagnosing and treating nosocomial pneumonia has been estimated at greater than $2 billion (Craven, D. E. et al. (1991) Am J Med. 91(3B):44S-53S).

Treatment of these so-called nosocomial infections is complicated by the fact that bacteria encountered in hospital settings are often resistant to many antibiotics. In June 1998, the National Nosocomial Infections Surveillance (NNIS) System reported increases in resistance of P. aeruginosa isolates from intensive care units of 89% for quinolone resistance and 32% for imipenem resistance compared to the years 1993-1997 (Centers for Disease Control website). In fact, some strains of P. aeruginosa are resistant to over 100 antibiotics (Levy, S. (1998) Scientific American. March). There is a critical need to overcome the emergence of bacterial strains that are resistant to conventional antibiotics (Travis, J. (1994) Science 264:360-362).

P. aeruginosa is also of great industrial concern (Bitton, G. (1994) Wastewater Microbiology. Wiley-Liss, New York, N.Y.; Steelhammer, J. C. et al. (1995) Indust. Water Treatm.:49-55). The organism grows in an aggregated state, the biofilm, which causes problems in many water processing plants. Of particular public health concern are food processing and water purification plants. Problems include corroded pipes, loss of efficiency in heat exchangers and cooling towers, plugged water injection jets leading to increased hydraulic pressure, and biological contamination of drinking water distribution systems (Bitton, G. (1994) Wastewater Microbiology. Wiley-Liss, New York, N.Y., 9). The elimination of biofilms in industrial equipment has so far been the province of biocides. Biocides, in contrast to antibiotics, are antimicrobials that do not possess high specificity for bacteria, so they are often toxic to humans as well. Biocide sales in the US run at about $1 billion per year (Peaff, G. (1994) Chem. Eng. News:15-23).

### SUMMARY OF THE INVENTION

The present invention pertains to the modulation, e.g., inhibition, or the prevention of biofilm formation or development by a cell. The invention further pertains to methods for identifying modulators, e.g., inhibitors, of biofilm formation in bacteria, such as the human pathogen Pseudomonas aeruginosa. The invention also pertains to the modulation of antibiotic resistance in bacteria, e.g., Pseudomonas aeruginosa.

The inhibition of biofilm formation renders a bacterial population more susceptible to treatment, either directly through the host immune-response or in combination with traditional antibacterial agents and biocides. The present invention is based, at least in part, on the discovery that certain genes are differentially expressed in biofilm forming bacteria versus non-biofilm forming (planktonic) bacteria.

Thus, in one aspect, the invention provides a method for identifying a compound capable of modulating biofilm formation by bacteria, e.g., in a subject, or biofouling, comprising contacting a biofilm-associated gene or polypeptide comprising the nucleotide or amino acid sequence of any of the genes or polypeptides listed in Table 1 with a test compound, and assaying the ability of the compound to modulate the expression of a biofilm-associated gene or the activity of a biofilm-associated polypeptide comprising the nucleotide sequence of any of the genes or polypeptides listed in Table 1. In one embodiment, the compound inhibits or prevents biofilm formation or biofouling. In another embodiment, the compound is a small molecule.

In another aspect, the invention provides a method for identifying a compound capable of modulating bacterial antibiotic resistance, comprising assaying the ability of the compound to modulate the expression of a biofilm-associated gene or the activity of a biofilm-associated polypeptide comprising the nucleotide sequence of any of the genes or polypeptides listed in Table 2. In one embodiment, the antibiotic is tobramycin. In another embodiment, the bacteria is Pseudomonas aeruginosa.

Another aspect of the invention provides a method of assessing or diagnosing whether a subject is afflicted with a biofilm-associated disease or disorder, the method comprising comparing the level of expression of a biofilm-associated gene or the activity of a biofilm-associated polypeptide in a subject sample, e.g., a lung tissue sample, wherein the biofilm-associated gene or polypeptide is selected from the group consisting of the biofilm-associated genes and polypeptides listed in Table 1, and the level of expression of the biofilm-associated gene or the activity of a biofilm-associated polypeptide in a control non-biofilm producing bacterial sample, wherein differential expression of the biofilm-associated gene in the subject sample compared to the non-biofilm producing bacterial sample is an indication that the patient is afflicted with a biofilm-associated disease or disorder and wherein altered polypeptide activity of the biofilm-associated gene in the subject sample compared to the non-biofilm producing bacterial sample is an indication that the patient is afflicted with a biofilm-associated disease or disorder. In one embodiment, the subject is human. In another embodiment, the subject is immunocompromised. In yet another embodiment, the biofllm-associated disease or disorder is selected from the group consisting of cystic fibrosis, AIDS, middle ear infections, acne, periodontal disease, catheter-associated infections or medical device-associated infections. In a further embodiment, the non-biofilm producing bacterial sample is Pseudomonas aeruginosa. In a further embodiment, the biofilm producing bacterial sample is Pseudomonas aeruginosa.

Yet another aspect of the invention provides a method of detecting the presence of biofilm or biofilm forming bacteria, e.g., on the surface or within a medical device, the method comprising comparing the level of expression of a biofilm-associated gene or the activity of a biofilm-associated polypeptide in a sample, wherein the biofilm-associated gene or polypeptide is selected from the group consisting of the biofilm-associated genes and polypeptides listed in Table 1, and the level of expression of the biofilm-associated gene or the activity of a biofilm-associated polypeptide in a control non-biofilm producing bacterial sample, wherein differential expression of the biofilm-associated gene in the sample compared to the non-biofilm producing bacterial sample is an indication that biofilm and/or biofilm producing bacteria are present, and wherein altered polypeptide activity of the biofilm-associated gene in the sample compared to the non-biofilm producing bacterial sample is an indication that biofilm and/or biofilm producing bacteria are present. In one embodiment, the non-biofilm producing bacterial sample is Pseudomonas aeruginosa. In another embodiment, the biofilm producing bacterial sample is Pseudomonas aeruginosa.

In yet another aspect, the invention provides a method for treating a subject having a biofilm-associated disease or disorder by administering to the subject a therapeutically effective amount of a biofilm-associated nucleic acid or polypeptide modulator. In one embodiment, the biofilm-associated polypeptide modulator is selected from the group consisting of a small molecule, an antibody specific for a biofilm-associated polypeptide, a biofilm-associated polypeptide, e.g., a biofilm-associated polypeptide comprising the amino acid sequence of any one of SEQ ID NOs:86-158, a biofilm-associated polypeptide comprising an amino acid sequence which is at least 50%, 60%, 70%, 75%, 80%, 85%, 90%, 9%, 96%, 97%, 98%, 99%, or more identical to any one of the amino acid sequences of SEQ ID NOs:86-158, or a fragment of a biofilm-associated polypeptide comprising an amino acid sequence of any one of SEQ ID NOs:86-158. In another embodiment, the biofilm-associated nucleic acid modulator is selected from the group consisting of a biofilm-associated nucleic acid molecule comprising, e.g., the nucleotide sequence of any one of SEQ ID NOs:1-73, a biofilm-associated nucleotide comprising a nucleic acid sequence which is at least 50%, 60%, 70%, 75%, 80%, 85%, 90%, 95%, 96%, 97%, 98%, 99%, or more identical to any one of the nucleic acid sequences of SEQ ID NOs:1-73, or a fragment of a biofilm-associated nucleotide comprising a nucleotide sequence of any one of SEQ ID NOs:1-73, an antisense biofilm-associated nucleic acid molecule, and a ribozyme. In still another embodiment, the biofilm-associated associated nucleic acid or protein modulator is administered in a pharmaceutically acceptable formulation. In still another embodiment, the biofilm-associated nucleic acid modulator is administered using a gene therapy vector.

In yet another aspect, the invention provides a method for modulating, e.g., inhibiting, or preventing biofilm formation by contacting a biofilm-forming cell, e.g., a bacterial cell, with a biofilm-associated nucleic acid modulator or biofilm-associated polypeptide modulator, thereby modulating biofilm formation. In one embodiment, the biofilm-associated nucleic acid modulator or biofilm-associated polypeptide modulator may be used in combination with traditional antibacterial agents and biocides known or used in the art.

In a further aspect, the invention provides a method for identifying a compound capable of modulating, e.g., inhibiting, or preventing the formation of biofilm by contacting a nucleic acid molecule comprising the nucleotide sequence of any one of SEQ ID NOs.:1-73 or a polypeptide comprising the amino acid sequence of any one of SEQ ID NOs:86-158, with a test compound, and assaying the ability of the compound to modulate nucleic acid expression of a nucleotide comprising the nucleotide sequence of any one of SEQ ID NOs.:1-73 or polypeptide activity of a polypeptide comprising the amino acid sequence of any one of SEQ ID NOs:86-158.

In still a further aspect, the invention provides a method for identifying a compound capable of modulating antibiotic resistance by bacteria comprising contacting a nucleic acid molecule comprising the nucleotide sequence of any one of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, or SEQ ID NO: 71 with a test compound, and assaying the ability of the compound to modulate nucleic acid expression of any one of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, or SEQ ID NO: 71, thereby identifying a compound capable of modulating antibiotic resistance by bacteria. In one embodiment, the bacteria is Pseudomonas aeruginosa.

In still a further aspect, the invention provides a method for identifying a compound capable of modulating antibiotic resistance by bacteria comprising contacting a polypeptide comprising the amino acid sequence of any one SEQ ID NOs.:159-170, SEQ ID NO:89, SEQ ID NO:93, SEQ ID NO:109, SEQ ID NO:111, SEQ ID NO:133, SEQ ID NO:142, SEQ ID NO:141, or SEQ ID NO: 156 with a test compound, and assaying the ability of the compound to modulate polypeptide activity of any one of SEQ ID NOs.: SEQ ID NOs.:159-170, SEQ ID NO:89, SEQ ID NO:93, SEQ ID NO:109, SEQ ID NO:11, SEQ ID NO:133, SEQ ID NO:142, SEQ ID NO:141, or SEQ ID NO:156, thereby identifying a compound capable of modulating antibiotic resistance by bacteria. In one embodiment, the bacteria is Pseudomonas aeruginosa.

In another aspect, the invention provides methods for identifying biofilm regulated genes. The methods include comparing the expression of a bacterial gene from a cell growing in biofilm with the expression of a bacterial gene from a planktonic bacterial cell, wherein a gene which is differentially expressed in a cell growing in biofilm is a biofilm-regulated gene. In one embodiment the expression of a bacterial gene from a cell growing in biofilm and the expression of a bacterial gene from a planktonic bacterial cell is determined using a microarray. In another embodiment, the biofilm-regulated gene is regulated by exposure to an antibiotic.

In another aspect, the invention provides a method for identifying a compound capable of modulating, e.g., inhibiting, the formation of biofilm. The method includes contacting a cell with a test compound, wherein the cell expresses a gene comprising any one of SEQ ID NOs.:1-73 or a polypeptide comprising any one of SEQ ID NOs:86-158, and wherein the gene has been mutated such that the cell exhibits increased biofilm production compared to the wild-type cell; and determining the ability of the test compound to modulate, e.g., inhibit, biofilm formation by the cell containing the mutated gene as compared to the wild-type cell. In one embodiment, the mutated gene is a mutated rpoS gene.

In still another aspect, the invention provides a method for identifying a compound capable of modulating, e.g., decreasing, antibiotic resistance. The method includes contacting a cell with a test compound, wherein the cell expresses a gene comprising any one of SEQ ID NOs.:1-73 and wherein the gene has been mutated such that the cell exhibits increased antibiotic resistance compared to the wild-type cell; and determining the ability of the test compound to modulate, e.g., decrease, antibiotic sensitivity of the cell containing the mutated gene as compared to the wild-type cell, thereby identifying a compound capable of modulating antibiotic resistance of a cell. In one embodiment, the mutated gene is a mutated rpoS gene.

Other features and advantages of the invention will be apparent from the following detailed description and claims.

### DETAILED DESCRIPTION OF THE INVENTION

The present invention provides methods for the diagnostic evaluation of biofilm formation. The invention also provides methods for identifying a compound capable of modulating biofilm formation and antibiotic resistance. The present invention further provides methods for the identification and therapeutic use of compounds as treatments of biofilm-associated diseases or disorders. The present invention still further provides methods for modulating, e.g., inhibiting or preventing, biofilm formation, e.g., in a subject, and methods for modulating, e.g., inhibiting or preventing biofouling.

The invention is based, at least in part, on the discovery of bacterial (e.g., Pseudomonas aeruginosa) genes which are differentially expressed in biofilm forming bacterial populations (see Table 1). The invention is also based, in part, on the discovery of bacterial (e.g., Pseudomonas aeruginosa) genes which are differentially expressed in biofilms treated with an aminoglycoside antibiotic, e.g., tobramycin, versus untreated biofilms (see Table 2).

Bacteria often exist as sessile biofilm communities. Biofilm bacteria are resistant to antimicrobial treatments. Thus, biofilm infections with opportunistic pathogenic bacteria such as Pseudomonas aeruginosa are persistent. Therefore, the genes identified herein as being differentially expressed in biofilm forming bacterial populations and in biofilms treated with antibiotics compared to untreated biofilms, and the polypeptides encoded by these genes, provide targets for the modulation of biofilm formation and/or development as well as for the modulation of antibiotic resistance by bacteria. The SEQ ID NOs listed in Tables 1 and 2 and referred to herein correspond to the “PA” identification numbers listed in the Tables (e.g., SEQ ID NO:1 corresponds to the nucleotide sequence for gene identification number PA0723. The corresponding amino acid sequence is set forth as SEQ ID NO:86). The nucleotide and amino acid sequences of all of the genes and polypeptides listed in Tables 1 and 2 can be accessed via the Internet at the Pseudomonas Genome Project website.

Biofilm development appears to proceed through a number of programmed steps (J. W. Costerton, et al. Science 284, 1318 (1999); G. A. O'Toole, R. Kolter, Mol. Microbiol 30, 295 (1998)). Biofilm bacteria possess special characteristics such as antibiotic resistance, and bacteria in biofilms represent heterogeneous groups of cells exposed to different microenvironments. Biofilm formation by P. aeruginosa occurs in discrete steps: surface attachment and multiplication, microcolony formation, and differentiation into mature, structured antibiotic-resistant communities. Gene expression differences in a mature biofilm versus planktonic cells is particularly relevant because of the resistance of mature biofilms to antimicrobial treatment.

A P. aeruginosa microarray was used to compare gene expression in planktonic and biofilm forming bacterial cells (M. G. Bangera, J. K. Ichikawa, C. Marx, S. Lory, paper presented at American Society of Microbiology 100^{th} general meeting, Los Angeles, Calif., 2000). The present invention is not limited to the use of genes and polypeptides from P. aeruginosa. A small number of genes, 73, showed differential expression (at least a 2-fold difference, see Table 1 in Example 1, corresponding to SEQ ID NOs.:1-73). The proteins encoded by these genes are set forth as SEQ ID NOs:86-158. Thirty-four of these genes were activated and 39 were repressed in biofilm populations.

Genes for synthesis of pili and flagella are repressed in biofilms (Table 1). Pili and flagella have been reported to be involved in the initial steps (attachment and microcolony formation) of P. aeruginosa biofilm development (G. A. O'Toole, R. Kolter, Mol. Microbiol. 30, 295 (1998)).

These results suggest that these appendages may not be required for maintenance of a mature biofilm and that they are involved in committed steps in biofilm development. Once development has proceeded through these steps pili and flagella are no longer required.

These data show that none of the genes for synthesis of pili and flagella were induced in the biofllm. However, some of the genes that are activated or repressed in biofilms are known to affect antibiotic sensitivity in P. aeruginosa. Aminoglycosides like tobramycin and gentamicin are front-line antibiotics in the treatment of P. aeruginosa infections (N. Hoiby, “Pseudomonas in cystic fibrosis: past, present, and future” (Cystic Fibrosis Trust, 1998)). These cationic antibiotics bind to the negatively charged lipopolysaccharide (LPS) of the outer membrane (R. E. W. Hancock, Ann. Rev. Microbiol.38, 237 (1984); H. Nikaido, M. Vaara, Microbiol. Rev. 49, 872 (1985)), and subsequent transport into P. aeruginosa correlates with the level of the transmembrane electrical potential (L. E. Bryan, S. Kwan, Antimicrob. Agents Chemoth. 23, 835 (1983); L. E. Bryan, et al. Antimicrob. Agents Chemoth. 17, 71 (1980); P. D. Damper, W. Epstein, Antimicrob. Agents Chemoth. 20, 803 (1981)).

The major aminoglycoside-resistance mechanism of P. aeruginosa is impermeability of the bacteria to antibiotic entrance (L. E. Bryan, et al. J. Antibiot. (Tokyo) 29, 743 (1976); D. L. MacLeod, et al., J. Infect. Dis. 181, 1180 (2000)). This impermeability involves several factors including the tolA gene product (M. Rivera, et al. Antimicrob. Agents Chemoth. 32, 649 (1988)) and terminal electron transport proteins (L. E. Bryan, S. Kwan, Antimicrob. Agents Chemoth. 23, 835 (1983); L. E. Bryan, et al. Antimicrob. Agents Chemoth. 17, 71 (1980)). The tolA gene product affects LPS structure resulting in decreased aminoglycoside affinity for the outer membrane. Mutants that underproduce tolA are hypersensitive to aminoglycoside antibiotics (M. Rivera, et al. Antimicrob. Agents Chemoth. 32, 649 (1988)).

The tolA gene was activated in P. aeruginosa biofilms (Table 1). Biofilms exposed to tobramycin were compared with untreated biofilms (see Example 2). Twenty genes were differentially expressed in tobramycin-treated biofilms (Table 2, corresponding to SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, and SEQ ID NO: 71), 14 were activated and 6 were repressed by tobramycin (at 7× the minimum inhibitory concentration for planktonic cells). The proteins encoding these genes are set forth as SEQ ID NOs.:159-170, SEQ ID NO:89, SEQ ID NO:93, SEQ ID NO:109, SEQ ID NO:111, SEQ ID NO:133, SEQ ID NO:142, SEQ ID NO:141, and SEQ ID NO: 156. Of these 20 genes, 12 were classified as genes coding for hypothetical proteins of unknown function. Treatment with tobramycin, which causes errors in protein synthesis, appeared to induce a stress response with activation of dnaK and groES for example.

This analysis of P. aeruginosa biofilms shows that on average, gene expression in biofilm cells is remarkably similar to gene expression in planktonic cells maintained under similar environmental conditions. However, 73 genes were identified that were differentially expressed in biofilms. Involvement of all of these genes in biofilm resistance to antibiotics can be assessed. Moreover, some of these genes are involved in the maintenance of mature biofilms. The genes identified herein, and subsets thereof, are of great use in the development of rapid screens for agents or compounds that block biofilm maintenance, for diagnosis and/or of biofilm-associated disorders, and for treatment and/or prevention of biofouling.

Definitions

Before further description of the invention, certain terms employed in the specification, examples and claims are, for convenience, collected here.

The term “biofilm” is intended to include all biological films that are formed by microorganisms such as bacteria. Biofilms are composed of microorganisms, e.g., bacteria, embedded in an organic gelatinous structure composed of one or more matrix polymers which are secreted by the resident microorganisms. The language “biofilm development” or “biofilm formation” is intended to include the formation, growth, and modification of the bacterial colonies contained within the biofilm structures, as well as the synthesis and maintenance of the exopolysaccharide matrix of the biofilm structures.

The term “biofouling” refers to the undesirable formation and/or accumulation of biofilms on surfaces. For example, biofilms may form in industrial settings and lead to material degradation, product contamination, mechanical blockage, and impedance of heat transfer in water-processing systems. Biofouling also refers to biological contamination of water distribution systems, e.g., due to growth on surfaces such as, for example, filtration devices. Biofouling also refers to biofilm formation, for example, within food or on food processing devices, on medical devices, (e.g., catheters) or on the outside of vessels, e.g., boats or ships.

The term “biofilm-associated disease or disorder” includes diseases or disorders which are characterized by the presence or potential presence of a bio film, e.g., a bacterial biofilm. Biofilm-associated diseases or disorders include infection of the subject by one or more bacteria, e.g., Pseudomonas aeruginosa, Bacillus subtilis, Candida albicans, Staphylococcus aureus, Staphylococcus epidermidis, Enterococcus faecalis, Helicobacter pylori, Escherichia coli, Salmonella typhimurium, Legionella pneumophila, or other gram-negative or gram positive bacteria. Examples of biofilm-associated diseases or disorders include diseases or disorders caused by, for example, bacteria (e.g., gram-positive and/or gram-negative bacteria), fungi, viruses and parasites. Examples of biofilm-associated diseases or disorders include, but are not limited to, cystic fibrosis, AIDS, middle ear infections, osteomyelitis, acne, dental cavities, prostatitis, abscesses, bacteremia, contamination of peritoneal dialysis fluid, endocarditis, pneumonia, meningitis, cellulitis, pharyngitis, otitis media, sinusitis, scarlet fever, arthritis, urinary tract infection, laryngotracheitis, erysipeloid, gas gangrene, tetanus, typhoid fever, acute gastroenteritis, bronchitis, epiglottitis, plague, sepsis, chancroid, wound and burn infection, cholera, glanders, periodontitis, genital infections, empyema, granuloma inguinale, Legionnaire's disease, paratyphoid, bacillary dysentary, brucellosis, diphtheria, pertussis, botulism, toxic shock syndrome, mastitis, rheumatic fever, eye infections, including contact lens infections, periodontal infections, catheter- or medical device-associated infections, and plaque. Other biofilm-associated diseases or disorders include swine erysipelas, peritonitis, abortion, encephalitis, anthrax, nocardiosis, pericarditis, mycetoma, peptic ulcer, melioidosis, Haverhill fever, tularemia, Moko disease, galls (such as crown, cane and leaf), hairy root, bacterial rot, bacterial blight, bacterial brown spot, bacterial wilt, bacterial fin rot, dropsy, columnaris disease, pasteurellosis, furunculosis, enteric redmouth disease, vibriosis of fish, and fouling of medical devices.

The terms “biofilm-associated gene”, “biofilm-associated polypeptide”, or “biofilm-associated molecule”, refer to bacterial (e.g., Pseudomonas aeruginosa) genes which are differentially expressed in biofilm forming bacterial populations or the proteins encoded by these genes. These terms also refer to bacterial (e.g., Pseudomonas aeruginosa) genes (or the encoded proteins) which are differentially expressed in biofilms treated with an antibiotic, including, but not limited to, an aminoglycoside antibiotic (e.g., tobramycin or gentamicin), versus untreated biofilms. The terms biofilm-associated gene, protein, or molecule also refer to biofilm regulated genes, proteins, or molecules, which are regulated by the formation or development of biofilm by bacteria, e.g., Pseudomonas aeruginosa, or which are regulated by exposure to or treatment with antibiotics, e.g., aminoglycoside antibiotics (e.g., tobramycin or gentamicin). Examples of biofilm-associated molecules used in the methods of the invention include the nucleic acid molecules comprising the nucleotide sequence of SEQ ID NOs.: 1-85, and the proteins encoded by these nucleic acid sequences (SEQ ID NOs:86-170).

The term “modulator”, as in “modulator of biofilm formation” or “biofilm-associated gene modulator” or “biofilm-associated polypeptide modulator” is intended to encompass compounds capable of inducing and/or potentiating, as well as inhibiting and/or preventing biofilm-associated gene expression or biofilm-associated polypeptide activity. A modulator of biofilm formation may act to modulate either signal generation, signal reception (e.g., the binding of a signal molecule to a receptor or target molecule), signal transmission (e.g., signal transduction via effector molecules to generate an appropriate biological response), biofilm formation or development, or antibiotic resistance.

Modulators may be purchased, chemically synthesized or recombinantly produced. Modulators can be obtained from a library of diverse compounds based on a desired activity, or alternatively they can be selected from a random screening procedure. Examples of modulators include antibodies, polypeptides or fragments thereof, small molecules, nucleic acids or fragments thereof, or ribozymes. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule compounds depends upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the biofilm-associated molecule of the invention.

The terms “derived from” or “derivative”, as used interchangeably herein, are intended to mean that a sequence is identical to or modified from another sequence, e.g., a naturally occurring sequence. Derivatives within the scope of the invention include polynucleotide derivatives. Polynucleotide or nucleic acid derivatives differ from the sequences described herein (e.g., SEQ ID Nos.: 1-85) or known in nucleotide sequence. For example, a polynucleotide derivative may be characterized by one or more nucleotide substitutions, insertions, or deletions, as compared to a reference sequence. A nucleotide sequence comprising a biofilm-associated genetic locus that is derived from the genome of P. aeruginosa, e.g., SEQ ID NOs.:1-85, includes sequences that have been modified by various changes such as insertions, deletions and substitutions, and which retain the property of being regulated in response to biofilm formation or development or antibiotic resistance, or modulate biofilm formation or development or antibiotic resistance. The nucleotide sequence of the P. aeruginosa genome as well as the amino acid sequences of the proteins encoded by the P. aeruginosa genome are available at the Pseudomonas Genome Project website, and described in Stover, et al. (2000) Nature 406:959-964.

Polypeptide or protein derivatives include polypeptide or protein sequences that differ from the sequences described (SEQ ID NOs:86-170) in amino acid sequence, or in ways that do not involve the primary sequence, or both, and still preserve the activity of the polypeptide or protein. Derivatives of an amino acid sequence are produced when one or more amino acids is substituted with a different natural amino acid, an amino acid derivative or non-native amino acid. In certain embodiments protein derivatives include naturally occurring polypeptides or proteins, or biologically active fragments thereof, whose sequences differ from the wild type sequence by one or more conservative amino acid substitutions, which typically have minimal influence on the secondary structure and hydrophobic nature of the protein or peptide. Derivatives may also have sequences which differ by one or more non-conservative amino acid substitutions, deletions or insertions which do not abolish the biological activity of the polypeptide or protein.

In other embodiments, derivatives with amino acid substitutions which are less conservative may also result in desired derivatives, e.g., by causing changes in charge, conformation and other biological properties of the protein. Such substitutions would include, for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge. When the result of a given substitution cannot be predicted with certainty, the derivatives may be readily assayed according to the methods disclosed herein to determine the presence or absence of the desired characteristics. The polypeptides and proteins used in the methods of this invention may also be modified by various changes such as insertions, deletions and substitutions, either conservative or nonconservative where such changes might provide for certain advantages in their use.

The term “differential expression”, as used herein, includes both quantitative as well as qualitative differences in the expression of a gene. Thus, a differentially expressed gene may have its expression activated or inactivated in non-biofilm producing (planktonic) bacterial cells, versus cells growing in biofilms, e.g., Pseudomonas aeruginosa. The degree to which expression differs in planktonic versus cells growing in biofilms need only be large enough to be visualized via standard characterization techniques, e.g., microarray, quantitative PCR, Northern analysis, or subtractive hybridization. The expression of a differentially expressed gene may be used as part of a diagnostic evaluation to screen for biofilm formation or development, or may be used in methods for identifying compounds useful for the modulation of biofilm formation or development or antibiotic resistance. In addition, a differentially expressed gene involved in biofilm formation, e.g., a biofilm-associated gene, may represent a target gene such that modulation of the level of target gene expression or of target gene product activity may act to ameliorate a disease or disorder characterized by biofilm development or resistance to antibiotics caused by mature biofilm formation, e.g., biofilm-associated diseases or disorders. Moreover, a biofilm-associated gene may represent a target gene such that modulation of the level of target gene expression or of target gene product activity may act to ameliorate the formation of biofilm, e.g., in industrial facilities, e.g., biofouling of industrial water systems, or to treat a biofilm-associated disease or disorder.

As used herein, the term “genetic locus” includes a position on a chromosome, or within a genome, which is associated with a particular gene or genetic sequences having a particular characteristic. For example, in one embodiment, a biofilm-associated genetic locus includes nucleic acid sequences which comprise an open reading frame (ORF) of a biofilm-associated gene. Examples of biofilm-associated genetic loci of P. aeruginosa are described herein as SEQ ID NOs:1-85.

The term “operatively linked” or “operably linked” is intended to mean that molecules are functionally coupled to each other in that the change of activity or state of one molecule is affected by the activity or state of the other molecule. In one embodiment, nucleotide sequences are “operatively linked” when the regulatory sequence functionally relates to the DNA sequence encoding the polypeptide or protein of interest. For example, a nucleotide sequence comprising a transcriptional regulatory element(s) (e.g., a promoter) is operably linked to a DNA sequence encoding the protein or polypeptide of interest if the promoter nucleotide sequence controls the transcription of the DNA sequence encoding the protein of interest. In addition, two nucleotide sequences are operatively linked if they are coordinately regulated and/or transcribed. Typically, two polypeptides that are operatively linked are covalently attached through peptide bonds.

The term “regulatory sequences” is intended to include the DNA sequences that control the transcription of an adjacent gene. Gene regulatory sequences include, but are not limited to, promoter sequences that are found in the 5′ region of a gene proximal to the transcription start site which bind RNA polymerase to initiate transcription. Gene regulatory sequences also include enhancer sequences which can function in either orientation and in any location with respect to a promoter, to modulate the utilization of a promoter, and other expression control elements (e.g., polyadenylation signals). Such regulatory sequences are described, for example, in Goeddel (1990) Methods Enzymol. 185:3-7. Transcriptional control elements include, but are not limited to, promoters, enhancers, and repressor and activator binding sites.

The term “subject” includes organisms which are capable of suffering from biofilm-associated diseases or disorders. The term subject includes mammals, e.g., horses, monkeys, bears, dogs, cats, mice, rabbits, cattle, squirrels, rats, and, preferably, humans; plants, avian and aquatic organisms. In a further embodiment, the subject may be immunocompromised.

I. Screening Assays

The invention provides methods (also referred to herein as “screening assays”) for identifying modulators, i.e., candidate or test compounds or agents (e.g., peptides, peptidomimetics, small molecules (organic or inorganic) or other drugs) which bind to biofilm-associated polypeptides, have a stimulatory or inhibitory effect on, for example, biofilm-associated gene expression or biofilm-associated polypeptide activity, or have a stimulatory or inhibitory effect on, for example, the expression or activity of a biofilm-associated molecule substrate.

These assays are designed to identify compounds that bind to a biofilm-associated polypeptide, bind to other intracellular or extracellular proteins that interact with a biofilm-associated polypeptide, e.g., a polypeptide that participates in a signal transduction pathway that leads to biofilm formation, and interfere with the interaction of the biofilm-associated polypeptide with other cellular or extracellular proteins. A biofilm-associated polypeptide ligand can, for example, be used to modulate, e.g., inhibit, biofilm formation or development and/or antibiotic resistance. Such compounds may include, but are not limited to peptides, antibodies, or small organic or inorganic compounds. Such compounds may also include other cellular proteins.

Compounds identified via assays such as those described herein may be useful, for example, for modulating, e.g., inhibiting, biofilm formation or development and/or antibiotic resistance. In instances whereby biofilm formation or development and/or antibiotic resistance results from an overall lower level of biofilm-associated gene expression and/or biofilm-associated polypeptide, compounds that interact with the biofilm-associated polypeptide may include compounds which accentuate or amplify the activity of the biofilm-associated polypeptide. Such compounds would bring about an effective increase in the level of biofilm-associated molecule polypeptide activity, thus inhibiting biofilm formation or development and/or antibiotic resistance.

In other instances, mutations within the biofilm-associated gene may cause excessive amounts of biofilm-associated polypeptide to be made which leads to biofilm formation or development and/or antibiotic resistance. In such cases, compounds that bind to a biofilm-associated polypeptide may be identified that inhibit the activity of the biofilm-associated polypeptide. Assays for testing the effectiveness of compounds identified by techniques such as those described in this section are discussed herein.

In one embodiment, the invention provides assays for screening candidate or test compounds which are substrates of a biofilm-associated polypeptide or biologically active portion thereof. In another embodiment, the invention provides assays for screening candidate or test compounds which bind to or modulate the activity of a biofilm-associated polypeptide or biologically active portion thereof.

Test compounds, or modulators, can be exogenously added to cells growing in biofilms, produced by a second cell which is co-incubated with the cells growing in biofilms, or expressed by the cells growing in biofilms.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra).

In certain embodiments of the instant invention, the compounds tested are in the form of peptides from a peptide library. The peptide library may take the form of a cell culture, in which essentially each cell expresses one, and usually only one, peptide of the library. While the diversity of the library is maximized if each cell produces a peptide of a different sequence, it is usually prudent to construct the library so there is some redundancy. Depending on size, the combinatorial peptides of the library can be expressed as is, or can be incorporated into larger fusion proteins. The fusion protein can provide, for example, stability against degradation or denaturation. In an exemplary embodiment of a library for intracellular expression, e.g., for use in conjunction with intracellular target receptors, the polypeptide library is expressed as thioredoxin fusion proteins (see, for example, U.S. Pat. Nos. 5,270,181 and 5,292,646; and PCT publication WO94/02502). The combinatorial peptide can be attached on the terminus of the thioredoxin protein, or, for short peptide libraries, inserted into the so-called active loop.

In one aspect, the invention provides a method for identifying a compound capable of modulating, e.g., inhibiting or preventing, biofilm formation comprising contacting a nucleic acid or polypeptide molecule comprising the nucleotide sequence of any of the molecules listed in Table 1, with a test compound, and assaying the ability of the compound to modulate the expression of a nucleic acid molecule comprising the nucleotide sequence of any of the molecules listed in Table 1 or the activity of a polypeptide comprising the amino acid sequence of any of the molecules listed in Table 1. The biofilm formation, in a preferred embodiment, is by the human pathogen Pseudomonas aeruginosa.

Biofilm formation can be assessed by detection of bacterial signaling, e.g., signal molecules involved in quorum sensing signaling, or any signal transduction pathway that leads to biofilm formation (see, for example, Favre-Bonte, S., et al. (2002) Microb. Pathog. 32(3):143-7; Schaefer, et al. (2001) Methods Enzymol 336:41-7). Biofilm formation can also be assessed by detection of surface attachment by bacteria, use of scanning electron microscopy, use of transmission electron microscopy (TEM), by assessing other characteristics known to be typical of biofilm forming bacteria versus non-biofilm forming bacteria, or by other methods known in the art (see, for example, McFeters, et al.(1999)Symp Ser Soc Appl Microbiol 85(28):193S-200S. In addition, several methods are known in the art for detecting biofilm formation in, for example, medical devices (see, for example, Donlan, et al. (2001) J. Clin. Microbiology 39(2):750; Tunney, et al. (1999) Methods Enzymol 310:566; Merritt and Anderson (1998) J. Biomed. Res. 39(3):415). Identification of the components of the biofilm itself, e.g., polysaccharides present in the extracellular matrix, may also be utilized to detect the formation of a biofilm (see, for example, Leriche, V. et al. (2000) Appl. Environ Microbiol 66(5):1851-6); Baty, et al. (2001) Methods Enzymol 336:279-301; Giwereman, B. et al. (1992) FEMS Microbiol Immunol 4(4):225; Sugita, et al. (2001) cornea 20(4):362-5).

In another aspect, the present invention provides a method for identifying a compound capable of modulating, e.g., inhibiting, bacterial antibiotic resistance comprising contacting a nucleic acid molecule comprising the nucleotide sequence of any of the molecules listed in Table 2 with a test compound, and assaying the ability of the compound to modulate the expression of a nucleic acid molecule comprising the nucleotide sequence of any of the molecules listed in Table 2 or the activity of a polypeptide comprising the amino acid sequence of any of the molecules listed in Table 2. In one embodiment, the antibiotic is tobramycin. In another embodiment, the bacteria is Pseudomonas aeruginosa.

In a further aspect, the invention provides a method for identifying a compound capable of modulating, e.g., inhibiting, the formation of biofilm comprising contacting a nucleic acid molecule comprising the nucleotide sequence of any one of SEQ ID NOs.:1-73 or a polypeptide comprising the amino acid sequence of any one of SEQ ID NOs:86-158 with a test compound, and assaying the ability of the compound to modulate nucleic acid expression of any one of SEQ ID NOs.:1-73 or polypeptide activity of any one of SEQ ID NOs:86-158.

In still a further aspect, the invention provides a method for identifying a compound capable of modulating antibiotic resistance by bacteria comprising contacting a test compound with a nucleic acid molecule comprising the nucleotide sequence of any one of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, and SEQ ID NO: 71 or a polypeptide comprising the amino acid sequence of SEQ ID NOs.: SEQ ID NOs.: 159-170 and SEQ ID NO:89, SEQ ID NO:93, SEQ ID NO:109, SEQ ID NO:111, SEQ ID NO:133, SEQ ID NO:142, SEQ ID NO:141, and SEQ ID NO: 156, and assaying the ability of the compound to modulate nucleic acid expression of any one of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, and SEQ ID NO: 71, or polypeptide activity by any one of the polypeptides comprising the amino acid sequence of SEQ ID NOs.: SEQ ID NOs.:159-170 and SEQ ID NO:89, SEQ ID NO:93, SEQ ID NO:109, SEQ ID NO:11, SEQ ID NO:133, SEQ ID NO:142, SEQ ID NO:141, and SEQ ID NO: 156, thereby identifying a compound capable of modulating antibiotic resistance by bacteria. In one embodiment, the bacteria is Pseudomonas aeruginosa.

In another aspect, the invention provides a methods for identifying biofilm regulated genes comprising comparing the expression of a bacterial gene from a cell growing in biofilm versus the expression of a bacterial gene from a planktonic bacterial cell, wherein a gene which is differentially expressed in a cell growing in biofilm is a biofilm-regulated gene. In one embodiment the expression of a bacterial gene from a cell growing in biofilm and the expression of a bacterial gene from a planktonic bacterial cell is determined by microarray. In another embodiment, the biofilm-regulated gene is regulated by exposure to an antibiotic.

II. Diagnostic Assays for Biofilm Formation or Antibiotic Resistance

To determine whether a subject is afflicted with or prone to be afflicted with a biofilm-associated disease or disorder or whether antibiotic resistance exists in a subject, a biological sample may be obtained from a subject, e.g., a lung tissue sample, and the biological sample may be contacted with a compound or an agent capable of detecting a biofilm-associated polypeptide or nucleic acid (e.g., mRNA or genomic DNA) that encodes a biofilm-associated polypeptide, in the biological sample. A preferred agent for detecting biofilm-associated mRNA or genomic DNA is a labeled nucleic acid probe capable of hybridizing to biofilm-associated mRNA or genomic DNA. The nucleic acid probe can be, for example, the biofilm-associated nucleic acid set forth in any one of SEQ ID NOs:1-85, or a portion thereof, such as an oligonucleotide of at least 10, 15, 20, 25, 30, 25, 40, 45, 50, 100, 250 or 500 nucleotides in length and sufficient to specifically hybridize under stringent conditions to biofilm-associated mRNA or genomic DNA. Other suitable probes for use in the diagnostic assays of the invention are described herein.

A preferred agent for detecting biofilm-associated polypeptides in a sample is an antibody capable of binding to biofilm-associated polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect biofilm-associated mRNA, protein, or genomic DNA in a biological sample in vitro as well as in vivo. For example, in vitro techniques for detection of biofilm-associated mRNA include Northern hybridizations and in situ hybridizations. In vitro techniques for detection of biofilm-associated polypeptide include enzyme linked immunosorbent assays (ELISAs), Western blots, immunoprecipitations and immunofluorescence. In vitro techniques for detection of biofilm-associated genomic DNA include Southern hybridizations. Furthermore, in vivo techniques for detection of biofilm-associated polypeptide include introducing into a subject a labeled anti-biofilm-associated antibody. For example, the antibody can be labeled with a radioactive marker whose presence and location in a subject can be detected by standard imaging techniques.

In another embodiment, the methods further involve obtaining a control biological sample from a control subject, contacting the control sample with a compound capable of detecting biofilm-associated polypeptide, mRNA, or genomic DNA, such that the presence of biofilm-associated polypeptide, mRNA or genomic DNA is detected in the biological sample, and comparing the level of expression of biofilm-associated mRNA or genomic DNA or amount of biofilm-associated polypeptide, in the control sample with the level of expression of biofilm-associated mRNA or genomic DNA or amount of biofilm-associated polypeptide in the test sample.

To determine whether biofilm formation exists on any surface (e.g., on a medical device, such as a catheter, in a water distribution system or on a vessel), a sample of the surface may be analyzed using any of the techniques described herein.

III. Monitoring of Effects of Biofilm Modulators

The present invention further provides methods for determining the effectiveness of a biofilm-associated gene modulator or biofilm-associated polypeptide modulator in biofilm formation. For example, the effectiveness of a biofilm-associated modulator in increasing biofilm-associated gene expression, protein levels, or in upregulating biofilm-associated activity, can be monitored in clinical trials of subjects exhibiting decreased biofilm-associated gene expression, protein levels, or downregulated biofilm-associated activity. Alternatively, the effectiveness of a biofilm-associated modulator in decreasing biofilm-associated gene expression, protein levels, or in downregulating biofilm-associated activity, can be monitored in clinical trials of subjects exhibiting increased biofilm-associated gene expression, protein levels, or biofilm-associated activity. In such clinical trials, the expression or activity of a biofilm-associated gene, and preferably, other genes that have been implicated in, for example, a biofilm-associated disease or disorder can be used as a “read out” or marker of the phenotype of a particular cell.

For example, and not by way of limitation, genes, including biofilm-associated, that are modulated in cells by treatment with a compound which modulates biofilm-associated activity (e.g., identified in a screening assay as described herein) can be identified. Thus, to study the effect of compounds which modulate biofilm-associated activity on subjects suffering from a biofilm-associated disease or disorder in, for example, a clinical trial, cells can be isolated and RNA prepared and analyzed for the levels of expression of biofilm-associated and other genes implicated in the biofilm-associated disease or disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of biofilm-associated or other genes. In this way, the gene expression pattern can serve as a marker, indicative of the physiological response of the cells to the agent which modulates biofilm-associated activity. This response state may be determined before, and at various points during treatment of the individual with the agent which modulates biofilm-associated activity.

In a preferred embodiment, the present invention provides a method for monitoring the effectiveness of treatment of a subject with an agent which modulates biofilm-associated activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a biofilm-associated polypeptide, mRNA, or genomic DNA in the pre-administration sample; (iii) obtaining one or more post-administration samples from the subject; (iv) detecting the level of expression or activity of the biofilm-associated polypeptide, mRNA, or genomic DNA in the post-administration samples; (v) comparing the level of expression or activity of the biofilm-associated polypeptide, mRNA, or genomic DNA in the pre-administration sample with the biofilm-associated polypeptide, mRNA, or genomic DNA in the post administration sample or samples; and (vi) altering the administration of the agent to the subject accordingly. For example, increased administration of the agent may be desirable to increase the expression or activity of biofilm-associated to higher levels than detected, i.e., to increase the effectiveness of the agent. Alternatively, decreased administration of the agent may be desirable to decrease expression or activity of biofilm-associated to lower levels than detected, i.e. to decrease the effectiveness of the agent. According to such an embodiment, biofilm-associated gene or protein expression or activity may be used as an indicator of the effectiveness of an agent, even in the absence of an observable phenotypic response.

IV. Methods of Treatment of Subjects Suffering from Biofilm-Associated Disease or Disorders

The present invention provides for both prophylactic and therapeutic methods of treating a subject, e.g., a human, at risk of (or susceptible to) a biofilm-associated disease or disorder. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the biofihn-associated molecules of the present invention or biofilm-associated modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a biofilm-associated disease or disorder by administering to the subject an agent which modulates biofilm-associated gene expression or biofilm-associated polypeptide activity. Subjects at risk for a biofilm-associated disease or disorder can be identified by, for example, any or a combination of the diagnostic or prognostic assays described herein. Administration of a prophylactic agent can occur prior to the manifestation of symptoms characteristic of aberrant biofilm-associated gene expression or polypeptide activity, such that a biofilm-associated disease or disorder is prevented or, alternatively, delayed in its progression. Depending on the type of biofilm-associated aberrancy, for example, a biofilm-associated molecule agonist or biofilm-associated molecule antagonist agent can be used for treating the subject. The appropriate agent can be determined based on screening assays described herein.

B. Therapeutic Methods

Another aspect of the invention pertains to methods for treating a subject suffering from a biofilm-associated disease or disorder. These methods involve administering to a subject a biofilm-associated gene modulator or a biofilm-associated polypeptide modulator (e.g., a modulator identified by a screening assay described herein), or a combination of such modulators.

The agents or compounds which modulate biofilm formation can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates biofilm formation in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the agents that modulate biofilm formation are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates biofilm formation and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such biofilm modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates biofilm formation, as well as tailoring the dosage and/or therapeutic regimen of treatment with an agent which modulates biofilm formation.

Pharmacogenomics deals with clinically significant hereditary variations in the response to drugs due to altered drug disposition and abnormal action in affected persons. See, for example, Eichelbaum, M. et al (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a biofilm-associated polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2Cl9) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a biofilm-associated molecule or biofilm modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a biofilm-associated disease or disorder with an agent capable of modulation of biofilm formation.

V. Isolated Nucleic Acid Molecules

The methods of the invention include the use of isolated nucleic acid molecules (e.g., SEQ ID NOs.:1-85) that encode biofilm-associated polypeptides or biologically active portions thereof, as well as nucleic acid fragments sufficient for use as hybridization probes to identify biofilm-associated gene-encoding nucleic acid molecules (e.g., mRNA of biofilm-associated molecules) and fragments for use as PCR primers for the amplification or mutation of biofilm-associated nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used interchangeably herein, the terms “nucleic acid molecule” and “polynucleotide” are intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The term “DNA” refers to deoxyribonucleic acid whether single- or double-stranded. As used herein, the terms “gene” and “recombinant gene” refer to nucleic acid molecules which include an open reading frame encoding a protein, preferably a biofilm-associated polypeptide, and can further include non-coding regulatory sequences, and introns.

The present invention includes polynucleotides capable of hybridizing under stringent conditions, preferably highly stringent conditions, to the polynucleotides described herein (e.g., a biofilm-associated genetic locus, e.g., SEQ ID NOs.: 1-85). As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

The invention further encompasses nucleic acid molecules that differ from the biofilm-associated genetic loci described herein, e.g., the nucleotide sequences shown in SEQ ID NO:1-85. Accordingly, the invention also includes variants, e.g., allelic variants, of the disclosed polynucleotides or proteins; that is naturally occurring and non-naturally occurring alternative forms of the isolated polynucleotide which may also encode proteins which are identical, homologous or related to that encoded by the polynucleotides of the invention.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Allelic variants result, for example, from DNA sequence polymorphisms within a population (e.g., a bacterial population) that lead to changes in the nucleic acid sequences of biofilm-associated genetic loci.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at Accelrys™ website, formerly the Genetics Computer Group website), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at Accelrys™ website, formerly the Genetics Computer Group website), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4:11-17 (1988) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the National Center for Biotechnology Information website. Additionally, the “Clustal” method (Higgins and Sharp, Gene, 73:237-44, 1988) and “Megalign” program (Clewley and Arnold, Methods Mol. Biol, 70:119-29, 1997) can be used to align sequences and determine similarity, identity, or homology.

VI. Isolated Biofilm-Associated Polypeptides and Anti-Biofilm-Associated Antibodies

One aspect of the invention pertains to the use of isolated biofilm-associated polypeptides, and biologically active portions thereof, as well as the use of polypeptide fragments suitable for use as immunogens to raise anti-biofilm-associated antibodies. In one embodiment, native biofilm-associated polypeptides can be isolated from cells sources by an appropriate purification scheme using standard protein purification techniques. In another embodiment, biofilm-associated polypeptides are produced by recombinant DNA techniques. Alternative to recombinant expression, a biofilm-associated polypeptide can be synthesized chemically using standard peptide synthesis techniques.

An “isolated” or “purified” protein or biologically active portion thereof is substantially free of cellular material or other contaminating proteins from the cell or tissue source from which the biofilm-associated polypeptide is derived, or substantially free from chemical precursors or other chemicals when chemically synthesized. The language “substantially free of cellular material” includes preparations of biofilm-associated polypeptide in which the protein is separated from cellular components of the cells from which it is isolated or recombinantly produced. In one embodiment, the language “substantially free of cellular material” includes preparations of biofilm-associated polypeptide having less than about 30% (by dry weight) of non-biofilm-associated polypeptide (also referred to herein as a “contaminating protein”), more preferably less than about 20% of non-biofilm-associated polypeptide, still more preferably less than about 10% of non-biofilm-associated polypeptide, and most preferably less than about 5% non-biofilm-associated polypeptide. When the biofilm-associated polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of biofilm-associated polypeptide in which the protein is separated from chemical precursors or other chemicals which are involved in the synthesis of the protein. In one embodiment, the language “substantially free of chemical precursors or other chemicals” includes preparations of biofilm-associated polypeptide having less than about 30% (by dry weight) of chemical precursors or non-biofilm-associated chemicals, more preferably less than about 20% chemical precursors or non-biofilm-associated chemicals, still more preferably less than about 10% chemical precursors or non-biofilm-associated chemicals, and most preferably less than about 5% chemical precursors or non-biofilm-associated chemicals.

As used herein, a “biologically active portion” of a biofllm-associated polypeptide includes a fragment of a biofilm-associated polypeptide which participates in an interaction between a biofilm-associated molecule and a non-biofilm-associated molecule. Biologically active portions of a biofilm-associated polypeptide include peptides comprising amino acid sequences sufficiently identical to or derived from the amino acid sequence of the biofilm-associated polypeptide, e.g., the amino acid sequence shown in SEQ ID NOs:86-170, which include less amino acids than the full length biofilm-associated polypeptides, and exhibit at least one activity of a biofilm-associated polypeptide. Typically, biologically active portions comprise a domain or motif with at least one activity of the biofilm-associated polypeptide, e.g. modulating signal generation, signal reception, biofilm formation, biofilm development, or antibiotic resistance. A biologically active portion of a biofilm-associated polypeptide can be a polypeptide which is, for example, 10, 15, 20, 25, 30, 35, 40, 45, 50, 75, 100, 125, 150, 175, 200 or more amino acids in length. Biologically active portions of a biofilm-associated polypeptide can be used as targets for developing compounds which modulate biofilm formation.

In a preferred embodiment, the biofilm-associated polypeptide has an amino acid sequence shown in SEQ ID NOs:86-170. In other embodiments, the biofilm-associated polypeptide is substantially identical to SEQ ID NOs:86-170, and retains the functional activity of the protein of SEQ ID NOs:86-170, yet differs in amino acid sequence due to natural allelic variation or mutagenesis, as described in detail herein. Accordingly, in another embodiment, the biofilm-associated polypeptide is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to any one of SEQ ID NOs:86-170.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g. when aligning a second sequence to the biofilm-associated amino acid sequence of SEQ ID NOs:86-170 having 419 amino acid residues, at least 120, preferably at least 160, more preferably at least 201, even more preferably at least 241, and even more preferably at least 281 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at the Genetics Computer Group website), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at the Genetics Computer Group website), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to biofilm-associated nucleic acid molecules of the invention. BLAST protein searches can be performed with the XBLAST program, score=100, wordlength=3 to obtain amino acid sequences homologous to biofilm-associated protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the National Center for Biotechnology Information website.

The invention also provides biofilm-associated chimeric or fusion proteins. As used herein, a biofilm-associated “chimeric protein” or “fusion protein” comprises a biofilm-associated polypeptide operatively linked to a non-biofilm-associated polypeptide. A “biofilm-associated polypeptide” refers to a polypeptide having an amino acid sequence corresponding to biofilm-associated, whereas a “non-biofilm-associated polypeptide” refers to a polypeptide having an amino acid sequence corresponding to a protein which is not substantially homologous to a biofilm-associated polypeptide, e.g., a protein which is different from a biofilm-associated polypeptide and which is derived from the same or a different organism. Within a biofilm-associated fusion protein the biofilm-associated polypeptide can correspond to all or a portion of a biofilm-associated polypeptide. In a preferred embodiment, a biofilm-associated fusion protein comprises at least one biologically active portion of a biofilm-associated polypeptide. In another preferred embodiment, a biofilm-associated fusion protein comprises at least two biologically active portions of a biofilm-associated polypeptide. Within the fusion protein, the term “operatively linked” is intended to indicate that the biofilm-associated polypeptide and the non-biofilm-associated polypeptide are fused in-frame to each other. The non-biofilm-associated polypeptide can be fused to the N-terminus or C-terminus of the biofilm-associated polypeptide.

For example, in one embodiment, the fusion protein is a GST-biofilm-associated fusion protein in which the biofilm-associated sequences are fused to the C-terminus of the GST sequences. Such fusion proteins can facilitate the purification of recombinant biofilm-associated.

In another embodiment, the fusion protein is a biofilm-associated polypeptide containing a heterologous signal sequence at its N-terminus. In certain host cells (e.g., mammalian host cells), expression and/or secretion of biofilm-associated polypeptide can be increased through use of a heterologous signal sequence.

The biofilm-associated fusion proteins of the invention can be incorporated into pharmaceutical compositions and administered to a subject in vivo. The biofilm-associated fusion proteins can be used to affect the bioavailability of a biofilm-associated substrate. Use of biofilm-associated fusion proteins may be useful therapeutically for the treatment of biofilm-associated diseases or disorders.

Moreover, the biofilm-associated-fusion proteins of the invention can be used as immunogens to produce anti-biofilm-associated antibodies in a subject, to purify biofilm-associated ligands and in screening assays to identify molecules which inhibit the interaction of biofilm-associated with a biofilm-associated substrate.

Preferably, a biofilm-associated chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A biofilm-associated molecule-encoding nucleic acid can be cloned into such an expression vector such that the fusion moiety is linked in-frame to the biofilm-associated polypeptide.

The present invention also pertains to the use of variants of the biofilm-associated polypeptides which function as either biofilm-associated polypeptide agonists (mimetics) or as biofilm-associated polypeptide antagonists. Variants of the biofilm-associated polypeptides can be generated by mutagenesis, e.g., discrete point mutation or truncation of a biofilm-associated polypeptide. An agonist of the biofilm-associated polypeptides can retain substantially the same, or a subset, of the biological activities of the naturally occurring form of a biofilm-associated polypeptide. An antagonist of a biofilm-associated polypeptide can inhibit one or more of the activities of the naturally occurring form of the biofilm-associated polypeptide by, for example, competitively modulating a bio film-associated polypeptide-mediated activity of a biofilm-associated polypeptide. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the biofilm-associated polypeptide.

In one embodiment, variants of a biofilm-associated polypeptide which function as either biofilm-associated molecule agonists (mimetics) or as biofilm-associated molecule antagonists can be identified by screening combinatorial libraries of mutants, e.g., truncation mutants, of a biofilm-associated polypeptide for biofilm-associated polypeptide agonist or antagonist activity. In one embodiment, a variegated library of biofilm-associated molecule variants is generated by combinatorial mutagenesis at the nucleic acid level and is encoded by a variegated gene library. A variegated library of biofilm-associated molecule variants can be produced by, for example, enzymatically ligating a mixture of synthetic oligonucleotides into gene sequences such that a degenerate set of potential biofilm-associated sequences is expressible as individual polypeptides, or alternatively, as a set of larger fusion proteins (e.g., for phage display) containing the set of biofilm-associated gene sequences therein. There are a variety of methods which can be used to produce libraries of potential biofilm-associated variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential biofilm-associated gene sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a biofilm-associated polypeptide coding sequence can be used to generate a variegated population of biofilm-associated fragments for screening and subsequent selection of variants of a biofilm-associated polypeptide. In one embodiment, a library of coding sequence fragments can be generated by treating a double stranded PCR fragment of a biofilm-associated coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the biofilm-associated polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of biofilm-associated polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify biofilm-associated variants (Arkin and Yourvan (1992) Proc. Natl. Acad. Sci. USA 89:7811-7815; Delgrave et al. (1993) Protein Engineering 6(3): 327-331).

In one embodiment, cell based assays can be exploited to analyze a variegated biofilm-associated molecule library. For example, a library of expression vectors can be transfected into a cell line which ordinarily responds to a biofilm-associated molecule ligand in a particular biofilm-associated ligand-dependent manner. The transfected cells are then contacted with a biofilm-associated molecule ligand and the effect of expression of the mutant on, e.g., modulation of biofilm formation or modulation of antibiotic resistance can be detected. Plasmid DNA can then be recovered from the cells which score for inhibition, or alternatively, potentiation of signaling by the biofilm-associated molecule ligand, and the individual clones further characterized.

An isolated biofilm-associated polypeptide, or a portion or fragment thereof, can be used as an immunogen to generate antibodies that bind biofilm-associated polypeptide using standard techniques for polyclonal and monoclonal antibody preparation. A full-length biofilm-associated polypeptide can be used or, alternatively, the invention provides antigenic peptide fragments of biofilm-associated for use as immunogens. The antigenic peptide of biofilm-associated polypeptide comprises at least 8 amino acid residues of the amino acid sequence shown in SEQ ID NOS:86-170 and encompasses an epitope of biofilm-associated such that an antibody raised against the peptide forms a specific immune complex with biofilm-associated polypeptide. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of biofilm-associated polypeptides that are located on the surface of the protein, e.g., hydrophilic regions, as well as regions with high antigenicity.

A biofilm-associated polypeptide immunogen typically is used to prepare antibodies by immunizing a suitable subject, (e.g., rabbit, goat, mouse or other mammal) with the immunogen. An appropriate immunogenic preparation can contain, for example, a recombinantly expressed biofilm-associated polypeptide or a chemically synthesized biofilm-associated polypeptide. The preparation can further include an adjuvant, such as Freund's complete or incomplete adjuvant, or similar immunostimulatory agent. Immunization of a suitable subject with an immunogenic biofilm-associated preparation induces a polyclonal anti-biofilm-associated antibody response.

Accordingly, another aspect of the invention pertains to the use of anti-biofilm-associated antibodies. The term “antibody” as used herein refers to immunoglobulin molecules and immunologically active portions of immunoglobulin molecules, i.e., molecules that contain an antigen binding site which specifically binds (immunoreacts with) an antigen, such as a biofilm-associated polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind biofilm-associated polypeptides. The term “monoclonal antibody” or “monoclonal antibody composition”, as used herein, refers to a population of antibody molecules that contain only one species of an antigen biofilm-associated polypeptide binding site capable of immunoreacting with a particular epitope of biofilm-associated polypeptide. A monoclonal antibody composition thus typically displays a single binding affinity for a particular biofilm-associated polypeptide with which it immunoreacts.

Polyclonal anti-biofilm-associated antibodies can be prepared as described above by immunizing a suitable subject with a biofilm-associated immunogen. The anti-biofilm-associated antibody titer in the immunized subject can be monitored over time by standard techniques, such as with an enzyme linked immunosorbent assay (ELISA) using immobilized biofilm-associated. If desired, the antibody molecules directed against biofilm-associated can be isolated from the mammal (e.g. from the blood) and further purified by well known techniques, such as protein A chromatography to obtain the IgG fraction. At an appropriate time after immunization, e.g., when the anti-biofilm-associated antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a biofilm-associated immunogen as described above, and the culture supernatants of the resulting hybridoma cells are screened to identify a hybridoma producing a monoclonal antibody that binds biofilm-associated.

Any of the many well known protocols used for fusing lymphocytes and immortalized cell lines can be applied for the purpose of generating an anti-biofilm-associated monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind biofilm-associated, e.g., using a standard ELISA assay.

Alternative to preparing monoclonal antibody-secreting hybridomas, a monoclonal anti-biofilm-associated antibody can be identified and isolated by screening a recombinant combinatorial immunoglobulin library (e.g., an antibody phage display library) with biofilm-associated to thereby isolate immunoglobulin library members that bind biofilm-associated. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-biofilm-associated antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-biofilm-associated antibody (e.g., monoclonal antibody) can be used to isolate biofilm-associated polypeptides by standard techniques, such as affinity chromatography or immunoprecipitation. An anti-biofilm-associated antibody can facilitate the purification of natural biofilm-associated from cells and of recombinantly produced biofilm-associated expressed in host cells. Moreover, an anti-biofilm-associated antibody can be used to detect biofilm-associated polypeptide (e.g., in a cellular lysate or cell supernatant) in order to evaluate the abundance and pattern of expression of the biofilm-associated polypeptide. Anti-biofilm-associated antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, p-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

VII. Recombinant Expression Vectors and Host Cells

The present invention also discloses recombinant vector constructs and recombinant host cells transformed with said constructs for use in the methods of the invention.

The term “vector” or “recombinant vector” is intended to include any plasmid, phage DNA, or other DNA sequence which is able to replicate autonomously in a host cell. As used herein, the term “vector” refers to a nucleic acid molecule capable of transporting another nucleic acid to which it has been linked. A vector may be characterized by one or a small number of restriction endonuclease sites at which such DNA sequences may be cut in a determinable fashion without the loss of an essential biological function of the vector, and into which a DNA fragment may be spliced in order to bring about its replication and cloning. A vector may further contain a marker suitable for use in the identification of cells transformed with the vector. Recombinant vectors may be generated to enhance the expression of a gene which has been cloned into it, after transformation into a host. The cloned gene is usually placed under the control of (i.e., operably linked to) certain control sequences or regulatory sequences, which may be either constitutive or inducible.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. Expression systems for both prokaryotic and eukaryotic cells are described in, for example, chapters 16 and 17 of Sambrook, J. et al. Molecular Cloning: A Laboratory Manual. 2^{nd} ed., Cold Spring Harbor Laboratory, Cold Spring Harbor Laboratory Press, Cold Spring Harbor, N.Y., 1989.

In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses a transcriptional regulatory protein but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. ((1985) Mol. Cell. Biol. 5:3251-3260) can be used.

In general, it may be desirable that an expression vector be capable of replication in the host cell. Heterologous DNA may be integrated into the host genome, and thereafter is replicated as a part of the chromosomal DNA, or it may be DNA which replicates autonomously, as in the case of a plasmid. In the latter case, the vector will include an origin of replication which is functional in the host. In the case of an integrating vector, the vector may include sequences which facilitate integration, e.g., sequences homologous to host sequences, or encoding integrases.

Appropriate cloning and expression vectors for use with bacterial, fungal, yeast, and mammalian cellular hosts are known in the art, and are described in, for example, Powels et al. (Cloning Vectors: A Laboratory Manual, Elsevier, New York, 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a poly-adenylation site, splice donor and acceptor sites, and transcriptional termination sequences.

The vectors of the subject invention may be transformed into an appropriate cellular host for use in the methods of the invention.

As used interchangeably herein, a “cell” or a “host cell” includes any cultivatable cell that can be modified by the introduction of heterologous DNA. As used herein, “heterologous DNA”, a “heterologous gene” or “heterologous polynucleotide sequence” is defined in relation to the cell or organism harboring such a nucleic acid or gene. A heterologous DNA sequence includes a sequence that is not naturally found in the host cell or organism, e.g., a sequence which is native to a cell type or species of organism other than the host cell or organism. Heterologous DNA also includes mutated endogenous genetic sequences, for example, as such sequences are not naturally found in the host cell or organism. Preferably, a host cell is one in which a biofilm-associated molecule, e.g, a gene with the nucleotide sequence of SEQ ID NOs.: l-86, initiates a biofilm formation or antibiotic resistance response which includes the regulation of other biofilm-associated genetic sequences and non-biofilm-associated genetic sequences.

A host cell of the present invention includes prokaryotic cells and eukaryotic cells. Prokaryotes include gram negative or gram positive organisms, for example, E. Coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a preferred embodiment, a host cell of the invention is a mutant strain of P. aeruginosa in which lasI and rhlI are inactivated.

Eukaryotic cells include, but are not limited to, yeast cells, plant cells, fungal cells, insect cells (e.g., baculovirus), mammalian cells, and cells of parasitic organisms, e.g., trypanosomes. Mammalian host cell culture systems include established cell lines such as COS cells, L cells, 3T3 cells, Chinese hamster ovary (CHO) cells, embryonic stem cells, and HeLa cells. Other suitable host cells are known to those skilled in the art. DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

Host cells comprising an isolated nucleic acid molecule of the invention (e.g., a biofilm-associated genetic locus operatively linked to a reporter gene) can be used in the methods of the instant invention to identify a modulator of biofilm formation or development or antibiotic resistance in bacteria.

The invention further illustrated by the following examples which should not be construed as limiting. The contents of all references, patents and published patent applications cited throughout this application, as well as the Sequence Listing and the sequences identified by the Pseudomonas Genome Project gene identification numbers, are incorporated herein by reference.

### EXAMPLES

### Example 1

### Detection of P. aeruginosa Gene Expression Levels Using Microarrays

This example describes the P. aeruginosa microarray used to identify differentially expressed genes in biofilm cells. The array contains 5,500 of the predicted 5,570 P. aeruginosa genes (G. Bangera, J. K. Ichikawa, C. Marx, S. Lory, paper presented at American Society of Microbiology 100^{th} general meeting, Los Angeles, Calif., 2000).

Planktonic bacteria were grown in a chemostat at near the maximum growth rate for P. aeruginosa. The growth medium and dilution rate was the same as with the chemostat culture of planktonic bacteria. P. aeruginosa was grown using continuous culture techniques with an effort to minimize differences in the conditions to which P. aeruginosa was exposed.

P. aeruginosa PAO1 was grown at 37° C. with aeration in chemostat vessels (100 ml of medium, dilution rate, 0.2 h⁻¹). The growth medium consisted of 0.5% NH₄Cl, 0.25% NaCl, 0.015% KH₂PO₄, 1.5% MOPS (pH, 7.0), and 0.015% casamino acids. For biofilms, the chemostat vessel contained 100 g of sterilized granite pebbles (approximately 70 pebbles). Vessels were inoculated with approximately 10⁶ P. aeruginosa cells. For biofilm cultures, cells were allowed to attach to the rocks for 24 h prior to initiating the flow of medium, after which flow was initiated at a high rate (100 ml/h). After 2 h at this flow rate, more than 99% of all unattached bacteria were washed away. Flow was then decreased to give a final dilution rate identical to that in the planktonic chemostat. After 5 days cell numbers in the biofilm and planktonic chemostats were similar (1010 cells) as determined with standard plate counting techniques. To remove biofilm bacteria for plate counting, rocks were vortexed for 2 minutes in 5 ml phosphate buffered saline. A small planktonic population was present in the biofilm reactors throughout the experiment, but never represented more than 0.1% of the total chemostat population.

RNA was isolated from planktonic and biofilm bacteria using the Trizol reagent (Life Technologies, Grand Island, N.Y.). Planktonic cells were pelleted by centrifugation (10,000 rpm for 10 min) at 4° C. For biofilm RNA isolation, rocks containing biofilm bacteria were rinsed with sterile medium and suspended in Trizol reagent. After vortexing for 1 min the rocks were removed and the remaining cell material was sonicated for 10 s. Insoluble material was removed by centrifugation and RNA was isolated as described above. Contaminating DNA was eliminated by DNase treatment, and RNA was isolated by phenol-chloroform extraction and precipitation with LiCl. cDNA probes were produced from RNA using random primers (NSNSNSNSNS) and Cy5-dCTP or Cy3-dCTP (Amersham, Buckinghamshire, UK) according to previously described procedures (J. K. Ichikawa, et al., Proc. Natl. Acad. Sci. U.S.A. 97, 9659 (2000)). To avoid complications associated with Cy5-dCTP and Cy3-dCTP incorporation rates into resulting cDNA, each RNA comparison was performed with both dye combinations on separate microarrays.

The microarrays were glass microscope slides containing representative gene-specific DNA fragments from 5,500 of the estimated 5,570 open reading frames (ORFs) of P. aeruginosa. The microarrays were printed with a Generation II Array printer (Molecular Dynamics), and the hybridized microarrays were images with a Generation II scanning confocal fluorescent microscope (Molecular Dynamics).

Biofilm samples were prepared for scanning electron microscopy by fixation in 2.5% glutaraldehyde, and they were stained with 1% osmium tetroxide. The samples were dehydrated in ethanol and hexamethyldisilizane, air-dried, mounted on aluminum stubs, and sputter coated with gold and palladium (60:40). Imaging was with a Hitachi S-4000 scanning electron microscope.

Scanning electron micrographs of a P. aeruginosa biofilm on the surface of a granite pebble revealed rod-shaped P. aeruginosa and strings of dehydrated EPS connecting bacterial cells.

Results of the microarray analysis revealed 73 genes that showed differential expression (e.g., at least a 2-fold difference in expression).

Table 1, below, contains each gene that was differentially expressed in biofilm cells (corresponding to SEQ ID NOs.: 1-73). Corresponding amino acid sequences are identified as SEQ ID NOs:86-158. The data represent results of two independent experiments (average of 8 individual comparisons±standard error of the mean, SE). Positive values represent activation, and negative values represent repression in biofilms. Spot intensity on the microarray was measured based on an average total spot fluorescence (average of 8 independent spots). Spot intensities below 1000 were not included in the analysis because of statistical variability. P. aeruginosa ORF numbers and homologies were obtained from the Pseudomonas Genome Project website. Classifications are based on those described by Stover et al. (Nature 406, 959 (2000)). Statistical analysis of microarray data was performed using previously described computer software (J. K. Ichikawa, et al., Proc. Natl. Acad. Sci. U.S.A. 97, 9659 (2000)). Northern blot analysis and ribonuclease protection assays were performed as outlined by the manufacturer (Ambion, Austin, Tex.) to verify microarray data for four activated and repressed genes. These techniques revealed fold regulations of 75±10, 3.6±0.7, −25±5, and −3.0±0.7 for PA4867, PA0971, PA2128, and PA1080 respectively, and highlighted by bold print in Table 1.

TABLE 1
Genes differentially expressed in P. aeruginosa biofilms.

| SEQ | | | Fold |
|---|---|---|---|
| ID NO. | | | activation ± |
| NT | AA | P. aeruginosa ORF (number) | SE |

Bacteriophage genes

| | | | |
|---|---|---|---|
| 1 | 86 | Coat protein B of bacteriophage Pf1 | 83.5 ± 10.3 |
| | | (PA0723) | |
| 2 | 87 | Hypothetical protein of bacteriophage Pf1 | 64.2 ± 5.6 |
| | | (PA0722) | |
| 3 | 88 | Helix destabilizing protein of bacteriophage | 35.2 ± 2.7 |
| | | Pf1 (PA0720) | |
| 4 | 89 | Hypothetical protein of bacteriophage Pf1 | 26.6 ± 4.1 |
| | | (PA0721) | |
| 5 | 90 | Protein of bacteriophage Pf1 (PA0718) | 22.6 ± 2.9 |
| 6 | 91 | Hypothetical protein from bacteriophage Pf1 | 14.6 ± 2.4 |
| | | (PA0727) | |
| 7 | 92 | Probable coat protein A of bacteriophage Pf1 | 10.1 ± 0.6 |
| | | (PA0724) | |
| 8 | 93 | Hypothetical protein of bacteriophage Pf1 | 9.9 ± 1.1 |
| | | (PA0725) | |
| 9 | 94 | Hypothetical protein of bacteriophage Pf1 | 8.9 ± 0.5 |
| | | (PA0726) | |

Motility and attachment

| | | | |
|---|---|---|---|
| 10 | 95 | Probable fimbrial protein (PA2128) | −16.5 ± 1.5 |
| 11 | 96 | Pilin protein PilA (PA4525) | −6.6 ± 0.8 |
| 12 | 97 | Flagellar basal-body rod modification protein | −2.7 ± 0.3 |
| | | FlgD (PA1079) | |
| 13 | 98 | Probable pili assembly chaperone (PA2129) | −2.4 ± 0.2 |
| 14 | 99 | Flagellin type B (PA1092) | −2.3 ± 0.3 |
| 15 | 100 | Flagellar capping protein FliD (PA1094) | −2.1 ± 0.3 |
| 16 | 101 | Flagellar hook protein FlgE (PA1080) | −2.0 ± 0.1 |

Translation

| | | | |
|---|---|---|---|
| 17 | 102 | 50S ribosomal protein L28 (PA5316) | 4.4 ± 0.5 |
| 18 | 103 | 50S ribosomal protein L19 (PA3742) | 2.7 ± 0.1 |
| 19 | 104 | 50S ribosomal protein L4 (PA4262) | 2.4 ± 0.2 |
| 20 | 105 | 50S ribosomal protein L18 (PA4247) | 2.3 ± 0.3 |
| 21 | 106 | 50S ribosomal protein L23 (PA4261) | 2.3 ± 0.2 |
| 22 | 107 | 30S ribosomal protein S7 (PA4267) | 2.2 ± 0.3 |
| 23 | 108 | Translation initiation factor IF-2 (PA4744) | 2.1 ± 0.1 |
| 24 | 109 | Ribosome modulation factor (PA3049) | −5.3 ± 0.7 |
| 25 | 110 | ATP-binding protease component ClpA | −2.1 ± 0.1 |
| | | (PA2620) | |

Metabolism

| | | | |
|---|---|---|---|
| 26 | 111 | Urease beta subunit (PA4867) | 63.1 ± 8.1 |
| 27 | 112 | Ferredoxin [4Fe-4S] (PA0362) | 2.9 ± 0.3 |
| 28 | 113 | Lipoate-protein ligase B (PA3997) | 2.8 ± 0.4 |
| 29 | 114 | Glycerol-3-phosphate dehydrogenase | −4.1 ± 0.3 |
| | | (PA3584) | |
| 30 | 115 | Cytochrome c oxidase, subunit III (PA0108) | −2.9 ± 0.3 |
| 31 | 116 | Cytochrome c oxidase, subunit II (PA0105) | −2.9 ± 0.2 |
| 32 | 117 | Cytochrome c oxidase, subunit I (PA0106) | −2.7 ± 0.2 |
| 33 | 118 | Leucine dehydrogenase (PA3418) | −2.5 ± 0.2 |

Membrane Proteins/Secretion

| | | | |
|---|---|---|---|
| 34 | 119 | Translocation protein TatB (PA5069) | 6.9 ± 1.4 |
| 35 | 120 | TolA protein (PA0971) | 3.9 ± 0.4 |
| 36 | 121 | Translocation protein TatA (PA5068) | 2.4 ± 0.2 |
| 37 | 122 | Outer membrane lipoprotein OmlA (PA4765 | 2.4 ± 0.7 |
| 38 | 123 | Probable porin (PA3038) | −3.5 ± 0.5 |
| 39 | 124 | Exoenzyme S synthesis protein C precursor | −2.5 ± 0.3 |
| | | (PA1710) | |
| 40 | 125 | Probable sodium:solute symporter (PA3234) | −2.3 ± 0.1 |

Regulation

| | | | |
|---|---|---|---|
| 41 | 126 | Probable transcriptional regulator (PA2547) | 3.1 ± 0.1 |
| 42 | 127 | Sigma factor RpoH (PA0376) | 2.3 ± 0.3 |
| 43 | 128 | Sigma factor RpoS (PA3622) | −2.3 ± 0.3 |
| 44 | 129 | Probable two-component response regulator | −2.2 ± 0.2 |
| | | (PA4296) | |

Conserved hypothetical

| | | | |
|---|---|---|---|
| 45 | 130 | Conserved hypothetical protein (PA0990) | 4.0 ± 0.4 |
| 46 | 131 | Conserved hypothetical protein (PA0579) | 3.3 ± 0.7 |
| 47 | 132 | Conserved hypothetical protein (PA2971) | 2.4 ± 0.2 |
| 48 | 133 | Conserved hypothetical protein (PA3785) | −3.9 ± 0.5 |
| 49 | 134 | Conserved hypothetical protein (PA3235) | −3.2 ± 0.2 |
| 50 | 135 | Conserved hypothetical protein (PA4738) | −3.1 ± 0.2 |
| 51 | 136 | Conserved hypothetical protein (PA2621) | −3.0 ± 0.3 |
| 52 | 137 | Conserved hypothetical protein (PA0107) | −2.7 ± 0.2 |
| 53 | 138 | Conserved hypothetical protein (PA0588) | −2.6 ± 0.3 |
| 54 | 139 | Conserved hypothetical protein (PA1533) | −2.6 ± 0.3 |
| 55 | 140 | Conserved hypothetical protein (PA0587) | −2.3 ± 0.2 |

Hypothetical

| | | | |
|---|---|---|---|
| 56 | 141 | Hypothetical protein (PA1870) | 29.7 ± 1.2 |
| 57 | 142 | Hypothetical protein (PA3884) | 11.1 ± 1.6 |
| 58 | 143 | Hypothetical protein (PA3231) | 3.8 ± 0.4 |
| 59 | 144 | Hypothetical protein (PA0714) | 2.5 ± 0.3 |
| 60 | 145 | Hypothetical protein (PA1372) | 2.5 ± 0.3 |
| 61 | 146 | Hypothetical protein (PA3411) | −12.8 ± 1.7 |
| 62 | 147 | Hypothetical protein (PA1676) | −5.2 ± 0.6 |
| 63 | 148 | Hypothetical protein (PA1830) | −3.9 ± 0.9 |
| 64 | 149 | Hypothetical protein (PA4638) | −3.7 ± 0.8 |
| 65 | 150 | Hypothetical protein (PA1244) | −3.1 ± 0.4 |
| 66 | 151 | Hypothetical protein (PA1855) | −2.9 ± 0.2 |
| 67 | 152 | Hypothetical protein (PA4607) | −2.6 ± 0.3 |
| 68 | 153 | Hypothetical protein (PA4661) | −2.4 ± 0.2 |
| 69 | 154 | Hypothetical protein (PA3922) | −2.1 ± 0.2 |

Other

| | | | |
|---|---|---|---|
| 70 | 155 | Rod shape-determining protein MreC | 3.1 ± 0.5 |
| | | (PA4480) | |
| 71 | 156 | Probable DNA-binding protein (PA5348) | −4.6 ± 0.4 |
| 72 | 157 | Probable glycosyl hydrolase (PA2160) | −2.3 ± 0.2 |
| 73 | 158 | Methylated-DNA-protein-cysteine | −2.1 ± 0.2 |
| | | methyltransferase (PA0995) | |

Most P. aeruginosa genes were not differentially expressed in biofilm populations compared to non-biofilm populations. About 0.5% of genes were activated and about 0.5% were repressed in biofilms. Some of the activated genes are known to effect antibiotic sensitivity of non-biofilm-grown P. aeruginosa. Exposure of biofilms to the antibiotic tobramycin caused differential expression of 20 genes. The identification of biofilm-regulated genes points to mechanisms of biofilm resistance to antibiotics.

Most P. aeruginosa strain PAO1 genes were expressed at levels that allowed an analysis with reasonably small statistical variation (74% of the 5,500 genes arrayed). The great majority of these genes did not show differential expression in biofilm vs. planktonic cultures. This result argues strongly against the proposal that existence in a biofilm results in dramatic differences in the overall makeup of bacterial cells (J. W. Costerton, et al. Ann. Rev. Microbiol. 49, 711 (1995)). However, the present analysis averages gene expression in biofilms, which are heterogeneous groups of cells exhibiting different activities (J. W. Costerton, Z. Lewandowski, D. E. Caldwell, D. R. Korber, H. M. Lappin-Scott, Ann. Rev. Microbiol. 49, 711 (1995), M. G. Bangera, J. K. Ichikawa, C. Marx, S. Lory, paper presented at American Society of Microbiology 100^{th} general meeting, Los Angeles, Calif., 2000). It may be that certain subpopulations in the biofilm had substantially different patterns of gene expression than did the homogeneous planktonic population or the majority of metabolically active cells in the biofilm.

A small number of genes, 73, showed differential expression (at least a 2-fold difference, see Table 1, corresponding to SEQ ID NOs.:1-73). The proteins encoded by these genes are set forth as SEQ ID NOs:86-158. Thirty-four of these genes were activated and 39 were repressed in biofilm populations. The array data was validated by analyzing expression of several genes by Northern blotting and ribonuclease protection assays (Table 1). About 34% of the 73 biofilm-regulated genes code for hypothetical proteins of unknown function. This is slightly lower then the overall percentage of such genes (44%) derived from the P. aeruginosa genome sequencing project (C. K. Stover, et al., Nature 406, 959 (2000)).

The most highly activated genes in P. aeruginosa biofilms were those of the temperate filamentous bacteriophage Pf1. The P. aeruginosa PAO1 genome contains a nearly complete copy of the genome of bacteriophage Pf1 (11 of 14 genes present). The abundance of Pf1 in the fluid over the biofilms and in the planktonic chemostat culture fluid were assessed by plaquing on a Pf1-sensitive strain of P. aeruginosa. Pf1 concentrations were determined by plaque assay using P. aeruginosa PAK as the host bacterium. This bacteriophage produces small cloudy plaques typical of filamentous bacteriophage. The induced levels of Pf1 transcripts in biofilm cells were reflected by a 10²-10³-fold greater abundance of Pf1 in the biofilm reactor then in the plankton reactor. Phage induction might be important for gene transfer within biofilms, it could function in exclusion of other strains of P. aeruginosa from a biofilm, or perhaps as is the case for other temperate bacteriophage in other bacteria there is a toxin gene within the phage genome (J. B. Zabriskie, Annu. Rev. Med. 17, 337 (1966); J. A. Johnson, J. G. Morris, J. B. Kaper, J. Clin. Microbiol. 31, 732 (1993)).

Genes for synthesis of pili and flagella are repressed in biofilms (Table 1). Pili and flagella have been reported to be involved in the initial steps (attachment and microcolony formation) of P. aeruginosa biofilm development (G. A. O'Toole, R. Kolter, Mol. Microbiol. 30, 295 (1998)).

These results suggest that these appendages may not be required for maintenance of a mature biofllm. That they are involved in committed steps in biofilm development. Once development has proceeded through these steps pili and flagella are no longer required.

These data show that none of the genes for synthesis of pili and flagella were induced in the biofilm. However, some of the genes that are activated or repressed in biofilms are known to affect antibiotic sensitivity in P. aeruginosa. Aminoglycosides like tobramycin and gentamicin are front-line antibiotics in the treatment of P. aeruginosa infections (N. Hoiby, “Pseudomonas in cystic fibrosis: past, present, and future” (Cystic Fibrosis Trust, 1998)). These cationic antibiotics bind to the negatively charged lipopolysaccharide (LPS) of the outer membrane (R. E. W. Hancock, Ann. Rev. Microbiol.38, 237 (1984); H. Nikaido, M. Vaara, Microbiol. Rev. 49, 872 (1985)), and subsequent transport into P. aeruginosa correlates with the level of the transmembrane electrical potential (L. E. Bryan, S. Kwan, Antimicrob. Agents Chemoth. 23, 835 (1983); L. E. Bryan, et al. Antimicrob. Agents Chemoth. 17, 71 (1980); P. D. Damper, W. Epstein, Antimicrob. Agents Chemoth. 20, 803 (1981)).

### Example 2

### Detection of Genes Regulated by Tobramycin in P. aeruginosa Biofilms

This example describes the detection of genes regulated by tobramycin in P. aeruginosa biofilms.

The major aminoglycoside-resistance mechanism of P. aeruginosa is impermeability of the bacteria to antibiotic entrance (L. E. Bryan, et al. J. Antibiot. (Tokyo) 29, 743 (1976); D. L. MacLeod, et al., J. Infect. Dis. 181, 1180 (2000)). This impermeability involves several factors including the tolA gene product (M. Rivera, et al. Antimicrob. Agents Chemoth. 32, 649 (1988)) and terminal electron transport proteins (L. E. Bryan, S. Kwan, Antimicrob. Agents Chemoth. 23, 835 (1983); L. E. Bryan, et al. Antimicrob. Agents Chemoth. 17, 71 (1980)). The tolA gene product affects LPS structure resulting in decreased aminoglycoside affinity for the outer membrane. Mutants that underproduce tolA are hypersensitive to aminoglycoside antibiotics (M. Rivera, et al. Antimicrob. Agents Chemoth. 32, 649 (1988)).

The tolA gene was activated in P. aeruginosa biofilms (Table 1). Clearly this could contribute to the resistance of the biofilms to aminoglycosides. The cytochrome c oxidase genes were repressed. Cytochrome c oxidase is the terminal electron acceptor during aerobic growth, and repression of cytochrome c oxidase should decrease sensitivity of P. aeruginosa to aminoglycoside antibiotics (L. E. Bryan, S. Kwan, Antimicrob. Agents Chemoth. 23, 835 (1983); L. E. Bryan, et al. Antimicrob. Agents Chemoth. 17, 71 (1980)). These are just two examples of genes that may be involved in biofilm resistance to one class of antibiotics. Many other genes in Table 1 might be involved as well (for example the porin genes and the genes for alternate RNA polymerase σ factors). Of course the genes coding for proteins of unknown function are particularly interesting candidates as antibiotic-resistance factors. If such a gene is involved in antibiotic resistance, functional studies might reveal novel biofilm resistance mechanisms.

The existence in a biofilm might induce moderate levels of resistance to all antimicrobial treatments. This could afford cells in a biofilm the opportunity to respond to an antibiotic by inducing genes more specific to that antibiotic. Thus, biofilms exposed to tobramycin were compared with untreated biofilms. Tobramycin (5 μg/ml) was added to influent medium after 4 days of biofilm growth. After 24 h, biofilms were removed and processed for RNA. A 5 μg/ml concentration of tobramycin is approximately 7×the minimum inhibitory concentration of planktonic P. aeruginosa PAO1, but as indicated by plate counting 5 μg/ml tobramycin did not significantly affect cell numbers in biofilms.

Twenty genes were differentially expressed in tobramycin-treated biofilms (Table 2, corresponding to SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, and SEQ ID NO: 71), 14 were activated and 6 were repressed by tobramycin (at 7× the minimum inhibitory concentration for planktonic cells). The proteins encoding these genes are set forth as SEQ ID NOs.: 159-170, SEQ ID NO:89, SEQ ID NO:93, SEQ ID NO:109, SEQ ID NO:111, SEQ ID NO:133, SEQ ID NO:142, SEQ ID NO:141, and SEQ ID NO: 156. Of these 20 genes, 12 were classified as genes coding for hypothetical proteins of unknown function. As expected, treatment with tobramycin, which causes errors in protein synthesis, appeared to induce a stress response with activation of dnaK and groES for example. Of particular interest, tobramycin strongly induced several genes coding for hypothetical proteins. It also induced two probable efflux systems (a probable non-RND drug efflux system and a P-type ATPase). These are candidate tobramycin-resistance loci. Four genes that were induced in biofilms as compared to plankton were repressed by tobramycin treatment of biofilms, and two that were repressed in biofilms were activated in tobramycin treated biofilms.

The results (shown in Table 2, below) are from two independent experiments (1 standard error of the mean, SE). Positive values represent activation and negative values represent repression. Gene identifications and P. aeruginosa ORF numbers were obtained from the Pseudomonas Genome Project website. Genes that were repressed in biofilms and activated by tobramycin are underlined. Genes that were activated in biofilms and repressed by tobramycin are shown in italics and bold-face type. Eight of the 20 genes identified as being differentially expressed in tobramycin-treated biofilms (Table 2) were also identified as being differentially expressed in biofilms as compared to planktonic bacterial cells. These eight genes are identified by bold SEQ ID NOs in Table 2, below (e.g., SEQ ID NO:4, SEQ ID NO:8, SEQ ID NO:24, SEQ ID NO:26, SEQ ID NO:48, SEQ ID NO:57, SEQ ID NO:56, and SEQ ID NO: 71 and corresponding amino acid sequences set forth as SEQ ID NO:89, SEQ ID NO:93, SEQ ID NO:109, SEQ ID NO:111, SEQ ID NO:113, SEQ ID NO:142, SEQ ID NO:141, and SEQ ID NO:156).

TABLE 2
Genes regulated by tobramycin in P. aeruginosa biofilms

| SEQ | | | Fold |
|---|---|---|---|
| ID NO. | | | activation ± |
| NT | AA | P. aeruginosa ORF (number) | SE |
| 24 | 109 | Ribosome modulation factor (PA3049) | 24.7 ± 11.4 |
| 74 | 159 | Hypothetical protein (PA4326) | 23.2 ± 7.5 |
| 75 | 160 | Hypothetical protein (PA2703) | 19.3 ± 3.7 |
| 71 | 156 | Probable DNA binding protein (PA5348) | 18.1 ± 4.9 |
| 76 | 161 | Hypothetical protein (PA1110) | 8.7 ± 1.7 |
| 77 | 162 | Conserved hypothetical protein (PA3463) | 8.3 ± 3.2 |
| 78 | 163 | GroES (PA4386) | 5.8 ± 1.1 |
| 48 | 133 | Conserved hypothetical protein (PA3785) | 5.0 ± 1.3 |
| 79 | 164 | Probable drug efflux protein (PA1541) | 4.7 ± 1.4 |
| 80 | 165 | Probable metal transporting P-type ATPase | 3.6 ± 1.1 |
| | | (PA3920) | |
| 81 | 166 | Probable transcriptional regulator (PA3574) | 3.3 ± 0.6 |
| 82 | 167 | Conserved hypothetical protein (PA2498) | 2.9 ± 0.6 |
| 83 | 168 | DnaK (PA4761) | 2.7 ± 0.4 |
| 84 | 169 | Conserved hypothetical protein (PA3819) | 2.1 ± 0.3 |
| 85 | 170 | Hypothetical protein (PA1893) | −2.4 ± 0.3 |
| 8 | 93 | Hypothetical protein of bacteriophage PF1 | −3.1 ± 0.5 |
| | | (PA0725) | |
| 26 | 111 | Urease beta subunit (PA4867) | −3.7 ± 1.1 |
| 4 | 89 | Hypothetical protein of bacteriophage PF1 | −3.9 ± 1.0 |
| | | (PA0721) | |
| 56 | 141 | Hypothetical protein (PA1870) | −6.4 ± 1.5 |
| 57 | 142 | Hypothetical protein (PA3884) | −14.7 ± 5.7 |

### Example 3

### Effects of a Mutated Biofilm-Regulated Gene, rpoS, on Biofilm Formation and Antibiotic Resistance

If the microarray experiment described herein has identified genes important in biofilm development (Table 1) or antibiotic resistance (Table 2), then mutants defective in one or more of these genes should show aberrant biofilm structure and antibiotic sensitivity. In order to test this hypothesis, a mutant which was defective in one of the biofilm-regulated genes, rpoS (gene identification number PA3622, SEQ ID NO:43), was studied. The rpoS gene encodes for an RNA polymerase a subunit and influences transcription of other P. aeruginosa genes. Previous studies have indicated that an rpoS-deletion mutant of P. aeruginosa was hypervirulent in a mouse model, and that rpoS may serve a role in biofilm development (Suh, et al (1999) J. Bacteriology 181(13) 3890-7 and Heydom, et al. (2000) Microbiology 146:2409-15). Expression of the rpoS gene was repressed in the microarray experiment described herein (see Table 1).

To examine the involvement of rpoS in biofilm development, the wild-type strain and an isogenic rpoS mutant strain were grown in flow cell reactors and both were tagged with a gfp plasmid which expresses green fluorescent protein (GFP) (Whitely, et al. (2000) J. Bacteriology 182:4356-4360). The P. aeruginosa biofilms that developed in the flow cells were examined by scanning confocal laser microscopy. Within four hours, differences between the rpoS mutant and the wild-type biofilms were evident. The mutant had attached to and covered much more of the glass surface. In a quantitative microtitre dish assay, biofilm formation of the parent strain was 38% of the rpoS mutant. After 24 hours, the mutant biofilm had matured and large structured groups of bacteria were evident. The wild-type biofilm showed smaller structures and after a further incubation, the wild-type biofilm remained thinner than the mutant biofilm. This is consistent with a previous examination of an rpoS mutant: a 6 day-old rpoS mutant biofilm showed a mean thickness of 17 μm, and the thickness of the parent biofilm was 6 μm.

To assess whether rpoS influences the susceptibility of P. aeruginosa biofilms to antibiotics, biofilms of the P. aeruginosa wild-type and rpoS mutant were treated with increasing amounts of the aminoglycoside antibiotic tobramycin. Biofilms of the rpoS mutant were much more resistant to killing by tobramycin than were wild-type P. aeruginosa biofilms. These results indicate that the rpoS gene is important for biofilm formation and in susceptibility to the antibiotic tobramycin. These studies confirm that the microarray experiment, as described herein, identified a gene important for biofilm development and antibiotic susceptibility.

Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A method for identifying a compound capable of modulating biofilm formation by bacteria, comprising contacting a nucleic acid molecule comprising any one of the nucleotide sequences of SEQ ID NO:1-73 or a polypeptide comprising any one of the amino acid sequences of SEQ ID NO:86-158 with a test compound, and assaying the ability of the compound to modulate the expression of a nucleic acid molecule comprising any one of the nucleotide sequences of SEQ ID NO:1-73 or the activity of a polypeptide comprising any one of the amino acid sequences of SEQ ID NO:86-158, thereby identifying a compound capable of modulating biofilm formation by bacteria.

2. The method of claim 1, wherein the bacteria is Pseudomonas aeruginosa.

3. The method of claim 1, wherein said compound inhibits biofilm formation.

4. A method for identifying a compound capable of modulating bacterial antibiotic resistance, comprising contacting a nucleic acid molecule comprising any one of the nucleotide sequences of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, or SEQ ID NO: 71 or a polypeptide comprising any one of the amino acid sequences of SEQ ID NOs.:159-170, SEQ ID NO:89, SEQ ID NO:93, SEQ ID NO:109, SEQ ID NO:111, SEQ ID NO:133, SEQ ID NO:142, SEQ ID NO:141, or SEQ ID NO: 156 with a test compound, and assaying the ability of the compound to modulate the expression of a nucleic acid molecule comprising any one of the nucleotide sequences of SEQ ID NOs.:74-85, SEQ ID NO:4, SEQ ID NO:8, SEQ ID NO:24, SEQ ID NO:26, SEQ ID NO:48, SEQ ID NO:57, SEQ ID NO:56, or SEQ ID NO: 71 or the activity of a polypeptide comprising any one of the amino acid sequences of SEQ ID NOs.:159-170, SEQ ID NO:89, SEQ ID NO:93, SEQ ID NO:109, SEQ ID NO:111, SEQ ID NO:133, SEQ ID NO:142, SEQ ID No:141, or SEQ ID NO: 156, thereby identifying a compound capable of modulating bacterial antibiotic resistance.

5. The method of claim 4, wherein the bacteria is Pseudomonas aeruginosa.

6. The method of claim 4, wherein the antibiotic is tobramycin.

7. A method of assessing whether a subject is afflicted with a biofilm-associated disease or disorder, the method comprising comparing:
a) the level of expression of a biofilm-associated gene in a subject sample, wherein the biofilm-associated gene is selected from the group consisting of the biofilm-associated genes listed in Table 1, with
b) the level of expression of the biofilm-associated gene in a control non-biofilm producing bacterial sample,
wherein differential expression of the biofilm-associated gene in the subject sample compared to the non-biofilm producing bacterial sample is an indication that the patient is afflicted with a biofilm-associated disease or disorder, thereby assessing whether a subject is afflicted with a biofilm-associated disease or disorder.

8. The method of claim 7, wherein the subject is human.

9. The method of claim 7, wherein said subject is immunocompromised.

10. The method of claim 7, wherein said biofilm-associated disease or disorder is selected from the group consisting of cystic fibrosis, AIDS, middle ear infections, acne, periodontal disease, catheter-associated infections, and medical device-associated infections.

11. A method for treating a subject having a biofllm-associated disease or disorder comprising administering to the subject a therapeutically effective amount of a biofilm-associated nucleic acid modulator or biofilm-associated polypeptide modulator, thereby treating said subject having a biofilm-associated disease or disorder.

12. A method for modulating biofilm formation and development comprising contacting biofilm forming bacteria with an effective amount of a biofilm-associated nucleic acid modulator or a biofilm-associated polypeptide modulator, thereby modulating biofilm formation and development.

13. The method of claim 11 or 12, wherein the biofilm-associated polypeptide modulator is selected from the group consisting of a small molecule, an antibody specific for a biofilm-associated polypeptide, a biofilm-associated polypeptide, and a fragment of a biofilm-associated polypeptide.

14. The method of claim 11 or 12, wherein the biofilm-associated nucleic acid modulator is selected from the group consisting of a biofilm-associated nucleic acid molecule or protein, a fragment of a biofilm-associated nucleic acid molecule, an antisense biofilm-associated nucleic acid molecule, and a ribozyme.

15. The method of claim 11, wherein said biofilm-associated nucleic acid or biofilm-associated protein modulator is administered in a pharmaceutically acceptable formulation.

16. The method of claim 11 or 12, wherein said biofilm-associated polypeptide comprises the amino acid sequence of any of SEQ ID NOs:86-158, or a fragment thereof.

17. The method of claim 11 or 12, wherein said biofilm-associated nucleic acid modulator is administered using a gene therapy vector.

18. The method of claim 11 or 12, wherein said biofilm-associated nucleic acid molecule comprises the nucleotide sequence of any one of SEQ ID NOs:1-73 or a fragment thereof.

19. The method of claim 11, wherein the subject is a mammal.

20. The method of claim 11, wherein the subject is human.

21. The method of claim 11, wherein said subject is immunocompromised.

22. A method of identifying a biofilm-regulated gene comprising comparing the expression of a bacterial gene from a cell growing in biofilm with the expression of a bacterial gene from a planktonic bacterial cell, wherein a gene which is differentially expressed in a cell growing in biofilm is a biofilm-regulated gene.

23. The method of claim 22, wherein the expression of a bacterial gene from a cell growing in biofilm and the expression of a bacterial gene from a planktonic bacterial cell is determined by use of a microarray.

24. The method of claim 22, wherein the biofilm-regulated gene is regulated by exposure to an antibiotic.

25. A method for identifying a compound capable of modulating the formation of biofilm by a cell comprising:
a) contacting a cell with a test compound, wherein said cell expresses a gene comprising any one of SEQ ID NOs.:1-73 and wherein said gene has been mutated such that the cell exhibits increased biofilm production compared to a wild-type cell; and
b) determining the ability of the test compound to modulate biofilm formation by the cell containing the mutated gene as compared to the wild-type cell,
thereby identifying a compound capable of modulating the formation of biofilm.

26. The method of claim 25, wherein said mutated gene is a mutated rpoS gene.

27. A method for identifying a compound capable of modulating antibiotic resistance of a cell comprising:
a) contacting a cell with a test compound, wherein said cell contains a gene comprising any one of SEQ ID NOs.:1-73 and wherein said gene has been mutated such that the cell exhibits increased antibiotic resistance compared to a wild-type cell; and
b) determining the ability of the test compound to modulate antibiotic resistance of the cell containing the mutated gene as compared to a wild-type cell, thereby identifying a compound capable of modulating antibiotic resistance of a cell.

28. The method of claim 27, wherein said mutated gene is a mutated rpoS gene.
